(19) United States
(12) Patent Application Publication
(10) Pub. No.: US 2026/0001860 A1
(43) Pub. Date: Jan. 1, 2026

(54) METHODS AND COMPOSITIONS OF INHIBITING DCN1-UBC12 INTERACTION

(71) Applicant: University of Kentucky Research Foundation, Lexington, KY (US)

(72) Inventors: Leah C. Kovalic, Winchester, KY (US); Tucker J. Moseley, Darlington, IN (US); R. Kip Guy, Lexington, KY (US)

(21) Appl. No.: 18/874,832

(22) PCT Filed: Jun. 14, 2023
(86) PCT No.: PCT/US2023/025340

§ 371 Date: Dec. 13, 2024

Related U.S. Application Data
(60) Provisional application No. 63/352,221, filed on Jun. 14, 2022.

(51) Int. Cl. C07D 401/12; A61K 31/439; A61K 31/4468; A61K 31/454; A61K 31/4545; A61K 31/4709; A61K 31/4725; A61K 31/498; C07D 211/58; C07D 211/96; C07D 401/14; C07D 405/04; C07D 405/14; C07D 409/14; C07D 413/12; C07D 413/14; C07D 417/14; C07D 471/04; C07D 471/08

(52) U.S. Cl. CPC C07D 401/12; A61K 31/439; A61K 31/4468; A61K 31/454; A61K 31/4545; A61K 31/4709; A61K 31/4725; A61K 31/498; C07D 211/58; C07D 211/96; C07D 401/14; C07D 405/04; C07D 405/14; C07D 409/14; C07D 413/12; C07D 413/14; C07D 417/14; C07D 471/04; C07D 471/08

(57) ABSTRACT

A compound is provided that is a modified piperidinyl urea-based inhibitors of DCN1-UBE2M interaction, abridged or unbridged NAcM-OPT derivative compound, or a bridged and unbridged isoquinoline carboxamide derivative compound, or a pharmaceutically acceptable salt thereof. A pharmaceutical composition comprising the compound, or a pharmaceutically acceptable salt thereof, is provided. Methods for treating diseases or disorders characterized by uncontrolled cellular proliferation are described including administering the compound, pharmaceutically acceptable salt thereof, or pharmaceutical composition comprising the compound or pharmaceutically acceptable salt thereof to a subject in need thereof.

### GOVERNMENT INTEREST

This invention was made with government support under grant number ROI ca247365 awarded by the National Institutes of Health. The government has certain rights in the invention.

### TECHNICAL FIELD

The present invention relates to compounds for regulation of Ubiquitin-like protein (UBL) modification pathways. In particular, the presently-disclosed subject matter relates to small molecule regulation of UBL modification pathways, such as for the treatment of aberrant DCN1 expression associated with disorders characterized by unregulated cell proliferation.

### BACKGROUND

Ubiquitin-like protein (UBL) modification pathways have emerged as important targets for drug discovery based on their vast roles in regulation, and on clinical and preclinical successes of proteasome inhibitors (e.g. bortezomib or carfelzomib), E3 inhibitors, and the NEDD8 E1 inhibitor (MLN4924) (e.g., see Ciechanover, A. BioorgMed Chem 2013, 21:3400; and Bassermann, F.; Eichner, R.; Pagano, M. Biochim Biophys Acta 2014, 1843(1): 150-62). These chemical inhibitors of UBL pathways have also proven to be essential probes for dissecting multifactorial regulatory networks that were opaque to genetic approaches.

Currently, the FDA-approved drugs that target the ubiquitin-proteasome system (UPS), bortezomib and carfilzomib, completely block proteasome activity (see Petroski, M. D.; and Deshaies, R. J. Nat. Rev. Mol. Cell Biol. 2005, 6:9; da Silva, S. R, et al. J. Med. Chem. 2013, 56:2165; Hideshima, T., et al. Cancer Res. 2001, 61:3071; Shi, D.; and Grossman, S. R. Cancer Biol. Ther. 2010, 10:737). Clinically, this leads to toxicity; 50% of the patient population taking either proteasome inhibitor exhibit grade 3 hematologic adverse events (thrombocytopenia and neutropenia) and nearly 15% suffer grade 4 adverse events (potentially life-threatening) (see Jagannath, S., et al. Clin Lymphoma Myeloma Leuk 2012, 12:310; Siegel, D. S., et al, Blood 2012, 120:2817; Curran, M. P.; and McKeage, K. Drugs 2009, 69:859). However, there are no currently available therapeutic agents that specifically target components of the UBL system such as the DCN1-UBC12 interaction.

The function of DCN1 is to bind the acetylated N-terminus of UBC12 (an E2 enzyme for the UBL NEDD8) and the “Cullin (or CUL)” family of proteins to act as a co-E3 promoting NEDD8 modification (neddylation) of the CULs (see Kurz, T., et al. Mol. Cell 2008, 29:23; Scott, D. C, et al. Mol. Cell 2010, 39:784; Kim, A. Y. et. al. J. Biol. Chem. 2008, 283:33211; and Scott, D. C, et al. Science 2011, 334:674). DCN1 is part of a dynamic signaling system that regulates ligation of both NEDD8 and ubiquitin (UB), which are among more than a dozen human UBLs that dynamically post-translationally modify and regulate the functions of thousands of different eukaryotic proteins. It is believed that inhibition of the DCN1-UBC12 interaction could regulate CRL activity without completely blocking neddylation and provide efficacious compounds with less severe off-target effects and toxicity relative to existing drugs that target the UPS system (see Sun, Y. Neoplasia 2006, 8:645; Petroski, M. D.; and Deshaies, R. J. Nat Rev Mol Cell Biol 2005, 6:9; Nakayama, K. I.; and Nakayama, K. Nat Rev Cancer 2006, 6:369).

Despite advances in small molecule regulation of UBL modification pathways, there is still a scarcity of compounds that are potent, efficacious, and selective inhibitors of Cullin neddylation and also effective in the treatment of aberrant DCN1 expression associated with disorders characterized by unregulated cell proliferation, such as squamous cell carcinomas and other cancers and diseases in which the dysregulation of DCN1-dependent neddylation is involved. These needs and other needs are satisfied by the present invention.

### SUMMARY

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter relates in part to substituted 1-phenyl-3-(piperidin-4-yl)urea analogs, derivatives thereof, and related compounds, which are useful as inhibitors of the DCN1-UBC12 interaction, inhibitors of DCNT-mediated cullin-RING ligase activity, methods of making same, pharmaceutical compositions comprising same, methods of treating disorders using the disclosed compounds and compositions, methods of treating disorders associated with a DCN1-UBC12 interaction dysfunction, methods of treating disorders associated with a DCN1-mediated cullin-RING ligase activity dysfunction, methods of male contraception comprising the disclosed compounds and compositions, and kits comprising the disclosed compounds and compositions.

The presently-disclosed subject matter includes a compound having a structure as disclosed herein or a pharmaceutically acceptable salt thereof. Also disclosed are pharmaceutical compositions comprising a therapeutically effective amount of a disclosed compound, or a pharmaceutically acceptable salt thereof, and a pharmaceutically acceptable carrier.

Also disclosed are methods for the treatment of a disorder or disease characterized by uncontrolled cellular proliferation, comprising the step of administering to a mammal a therapeutically effective amount of a compound inhibiting DCN1-UBC12 interaction, wherein the compound is a disclosed compound.

Also disclosed are methods for the treatment of a neurodegenerative disorder, comprising the step of administering to a mammal a therapeutically effective amount of a compound inhibiting DCN1-UBC12 interaction, wherein the compound is a disclosed compound.

Also disclosed are methods for the treatment of a viral or bacterial infection, comprising the step of administering to a mammal a therapeutically effective amount of a compound inhibiting DCN1-UBC12 interaction, wherein the compound is a disclosed compound.

Also disclosed are methods for male contraception, comprising the step of administering to a mammal a therapeutically effective amount of a compound inhibiting DCN1-UBC12 interaction, wherein the compound is a disclosed compound.

Also disclosed are methods for inhibiting in at least one cell DCN1-mediated cullin-RING ligase activity, comprising the step of contacting the at least one cell with an effective amount of at least one compound of that is an inhibitor of DCN1-UBC12 interaction, wherein the compound is a disclosed compound.

Also disclosed are kits comprising at least one disclosed compound, or a pharmaceutically acceptable salt thereof; and one or more of: (a) at least one agent known to increase cell proliferation; (b) at least one agent known to increase activity of the ubiquitin-proteosome system; (c) at least one agent known to decrease activity of the ubiquitin-proteosome system; (d) at least one agent known to treat a disorder associated with DCN1-UBC12 interaction; or (e) at least one agent known to treat a disease characterized by uncontrolled cellular proliferation; or (f) instructions for treating a disease characterized by uncontrolled cellular proliferation.

Also disclosed are kits comprising at least one disclosed compound, or a pharmaceutically acceptable salt thereof; and one or more of: (a) at least one agent known to increase activity of the ubiquitin-proteosome system; (b) at least one agent known to decrease activity of the ubiquitin-proteosome system; (c) at least one agent known to treat a disorder associated with DCN1-UBC12 interaction; (d) at least one agent known to treat a neurodegenerative disease; or (e) instructions for treating a neurodegenerative disease.

Also disclosed are kits comprising at least one disclosed compound, or a pharmaceutically acceptable salt thereof; and one or more of: (a) at least one agent known to increase activity of the ubiquitin-proteosome system; (b) at least one agent known to decrease activity of the ubiquitin-proteosome system; (c) at least one agent known to treat a disorder associated with DCN1-UBC12 interaction; (d) at least one agent known to treat a viral or bacterial infection; or (e) instructions for treating a viral or bacterial infection.

Also disclosed are kits comprising at least one disclosed compound, or a pharmaceutically acceptable salt thereof; and one or more of: (a) at least one agent known to increase activity of the ubiquitin-proteosome system; (b) at least one agent known to decrease activity of the ubiquitin-proteosome system; (c) at least one agent known to treat a disorder associated with DCN1-UBC12 interaction; (d) at least one agent known to be used as a male contraceptive; or (e) instructions for effecting male contraception.

Also disclosed are uses of a disclosed compound, a disclosed product of making, or a pharmaceutically acceptable salt thereof, in the manufacture of a medicament for the treatment of a disorder associated with a DCN1-UBC12 interaction dysfunction.

Also disclosed are uses of a disclosed compound, a disclosed product of making, or a pharmaceutically acceptable salt thereof, in the manufacture of a medicament for the treatment of a disorder associated with a DCN1-mediated cullin-RING ligase activity dysfunction.

Also disclosed are uses of a disclosed compound, a disclosed product of making, or a pharmaceutically acceptable salt thereof, in the manufacture of a medicament for the male contraception.

Also disclosed are methods for the manufacture of a medicament to inhibit the DCN1-UBC12 interaction in a mammal comprising combining at least one disclosed compound or at least one disclosed product with a pharmaceutically acceptable carrier or diluent.

Also disclosed are methods for the manufacture of a medicament to inhibit DCN1-mediated cullin-RING ligase activity in a mammal comprising combining at least one disclosed compound or at least one disclosed product with a pharmaceutically acceptable carrier or diluent.

Also disclosed are methods for the manufacture of a medicament for male contraception in a mammal comprising combining at least one disclosed compound or at least one disclosed product with a pharmaceutically acceptable carrier or diluent.

In one aspect, the present disclosure is directed to a compound having a structure represented by a formula:

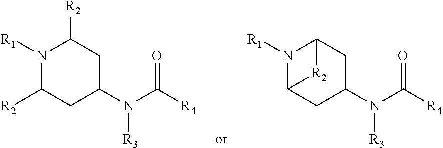

or a pharmaceutically acceptable salt thereof, wherein R₁ is alkyl, —SO₂-alkyl, or —CO-alkyl, R₂ is independently H, —CH₂, —CH₂—CH₂—, or —CH₃, R₃ is alkyl or —CH₂Ar, and R₄ is —NH—Ar, —NH-alkyl, Ar, or alkyl.

In one possible embodiment, R₂ is —CH₂—CH₂—. In another possible embodiment, R4 is —NH—Ar. In yet another possible embodiment, R4 is Ar. In other aspects, a pharmaceutical composition is disclosed comprising the above disclosed compound or a pharmaceutically acceptable salt thereof, and a pharmaceutically acceptable carrier. In still yet another embodiment, a method for treating a disorder characterized by uncontrolled cellular proliferation is disclosed, comprising the step of administering to a subject in need thereof a therapeutically effective amount of the above disclosed compound or a pharmaceutically acceptable salt thereof, or the above disclosed pharmaceutical composition.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E1Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders, e.g., a neurodegenerative disease or disease of uncontrolled cellular proliferation, associated with DCN1-UBC12 interaction prior to the administering step. In some aspects of the disclosed method, the subject has been diagnosed with a need for inhibition of DCN1-mediated cullin-RING ligase activity prior to the administering step. In some aspects of the disclosed method, the subject has been diagnosed with a disorder of uncontrolled cellular proliferation, e.g., a cancer, prior to the administering step. In some aspects of the disclosed method, the subject has been diagnosed with a neurodegenerative disorder prior to the administering step. In some aspects of the disclosed method, the subject has been identified with a disorder treatable by inhibiting the DCN1-UBC12 interaction prior to the administering step. In some aspects of the disclosed method, the subject has been identified with a disorder treatable by inhibiting DCN1-mediated cullin-RING ligase activity prior to the administering step. In some aspects of the disclosed method, the subject has been identified with a bacterial or viral infection prior to the administering step. In some aspects of the disclosed method, the subject has been identified with a need for male contraception. In one aspect, a subject can be treated prophylactically with a compound or composition disclosed herein, as discussed herein elsewhere.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by inhibiting the DCN1-UBC12 interaction” means having been subjected to a physical examination by a person of skill, for example, a physician, and found to have a condition that can be diagnosed or treated by a compound or composition that can inhibit the DCN1-UBC12 interaction. As a further example, “diagnosed with a need for inhibiting the DCN1-UBC12 interaction” refers to having been subjected to a physical examination by a person of skill, for example, a physician, and found to have a condition characterized by a DCN1-UBC12 interaction dysfunction. Such a diagnosis can be in reference to a disorder, such as a neurodegenerative disease, and the like, as discussed herein. For example, the term “diagnosed with a need for treatment of a disorder of uncontrolled cellular proliferation” refers to having been subjected to a physical examination by a person of skill, for example, a physician, and found to have a condition of uncontrolled cellular proliferation, e.g., a cancer, that can be treated by various therapeutic agents or methods, including, but not limited to, the disclosed compounds and/or products of the disclosed methods of making. For example, “diagnosed with a need for treatment of one or more disorders of uncontrolled cellular proliferation associated with a DCN1-UBC12 interaction dysfunction” means having been subjected to a physical examination by a person of skill, for example, a physician, and found to have one or more disorders of uncontrolled cellular proliferation, e.g., a cancer, associated with a DCN1-UBC12 interaction dysfunction.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to a DCN1-UBC12 interaction dysfunction) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intra-aural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, a target protein(s) (e.g., the DCN1-UBC12 proteins), or other biological entity together in such a manner that the compound can affect the activity of the target, either directly; e.g., by interacting with the target protein(s) itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “IC5₀,” is intended to refer to the concentration of a substance (e.g., a compound or a drug) that is required for 50% inhibition of a biological process, or component of a process, including a protein, subunit, organelle, ribonucleoprotein, etc. For example, IC5₀ refers to the half maximal (50%) inhibitory concentration (IC) of a substance as determined in a suitable assay. For example, an IC5₀ for inhibiting DCN1-UBC12 interaction can be determined in an in vitro assay system.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable mediajust prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group”, as used herein, denotes a hydrocarbon moiety that may be straight-chain (i.e., unbranched), branched, or cyclic (including fused, bridging, and spirofused poly cyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, \hat{ }-propyl, isopropyl, <<-butyl, isobutyl, s-butyl, i-butyl, <<-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group is acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alky Icy cloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcy cloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocy cloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A A⁴) are intended to include both the E and Z isomers. This can be presumed in structural formulae herein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the a clouds contain (4n+2)π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula-NA^{X}A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH².

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (teri-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A^{X}OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, 0, N, Si, P and S, the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, pyrazolopyrimidinyl, 1,2-oxazol-4-yl, 1,2-oxazol-5-yl, 1,3-oxazolyl, 1,2,4-oxadiazol-5-yl, 1,2,3-triazolyl, 1,3-thiazol-4-yl, pyridinyl, and pyrimidin-5-yl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle” and “poly cyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “tricyclic heterocycle” or “bicyclic heterocyclyl,” as used herein refers to a ring system in which at least one of the ring members is other than carbon. Bicyclic heterocyclyl encompasses ring systems an aromatic ring is fused with another aromatic ring, or an aromatic ring is fused with a non-aromatic ring. Bicyclic heterocyclyl encompasses ring systems a benzene ring is fused to a 5- or a 6-membered ring containing 1, 2 or 3 ring heteroatoms or a pyridine ring is fused to a 5- or a 6-membered ring containing 1, 2 or 3 ring heteroatoms.

Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A² A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula

A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “R”,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₁₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)o-iPh which may be substituted with R^{∘}; —CH═CHPh, which may be substituted with R^{∘}; —(CH₂)₀₋₄O(CH₂)₀-i-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)o ₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)o ₄C(O)R^{∘}; —C(S)R^{∘}; —(CH₂)o ₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR⁰₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR⁰²; —P(O)₂R^{∘}; —P(O)R^{∘}₂; —OP(O)R^{∘}₂; —OP(O)(OR^{∘})₂; SiR^{∘}₃; —(Ci₄ straight or branched alkylene)0-N(R^{∘})₂; or —(C₁₋₄ straight or branched alkylene)C(O)O—N(R^{∘})₂, each R^{∘} may be substituted as defined below and is independently hydrogen, C₁₋₆ aliphatic, —CH₂Ph, —O(CH₂)o-iPh, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)_{0 2}R^{●}, -(haloR^{●}), —(CH₂)0 2OH, —(CH₂)0 2OR^{●}, —(CH₂)_{0 2}CH(OR^{●})₂; —O(haloR^{●}), —CN, —N₃, —(CH₂)_{0 2}C(O)R^{●}, —(CH₂)_{0 2}C(O)OH, —(CH₂)0 2C(O)OR^{●}, —(CH₂)_{0 2}SR^{●}, —(CH₂)_{0 2}SH, —(CH₂)_{0 2}NH₂, —(CH₂)_{0 2}NHR^{●}, —(CH₂)_{0 2}NR^{●}₂, —NO₂, —SiR^{●}₃, —OSiR^{●}₃, —C(O)SR^{●}—(C₁₋₄ straight or branched alkylene)C(O)OR^{●}, or —SSR^{●} each R^{●} is unsubstituted or where preceded by “halo” is substituted only with one or more halogens, and is independently selected from C₁₋₄ aliphatic, —CH₂Ph, —O(CH₂)₀-iPh, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{●}₂, ═NNHC(O)R^{●}, ═NNHC(O)OR^{●}, ═NNHS(O)₂R^{●}, ═NR^{●}, ═NOR^{●}, —O(C(R^{●}₂))_{2 3}O— or —S(C(R^{●}₂))_{2_3}S—, each independent occurrence of R^{●} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{●}₂)₂ 3O—, each independent occurrence of R^{●} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, each R^{●} is unsubstituted or where preceded by “halo” is substituted only with one or more halogens, and is independently C₁₋₄ aliphatic, —CH₂Ph, —O(CH₂)₀-iPh, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, each R^{●} is unsubstituted or where preceded by “halo” is substituted only with one or more halogens, and is independently C₁₋₄ aliphatic, —CH₂Ph, —O(CH₂)₀ iPh, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons.

Examples of suitable leaving groups include halides and sulfonate esters, including, but not limited to, triflate, mesylate, tosylate, brosylate, and halides.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

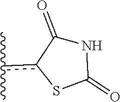

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which are added together prior to crystallization. In certain instances, the two or more molecules may owe their stability through non-covalent interaction. One or more components of this molecular complex provide a stable framework in the crystalline lattice. In certain instances, the guest molecules are incorporated in the crystalline lattice as anhydrates or solvates, see e.g., “Crystal Engineering of the Composition of Pharmaceutical Phases. Do Pharmaceutical Co-crystals Represent a New Path to Improved Medicines?” Almarasson, O., et al, The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

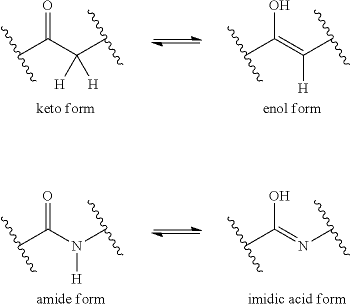 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N\hat{ }unsubstituted, 3-R³ and N\hat{ }unsubstituted, 5-R³ as shown below.

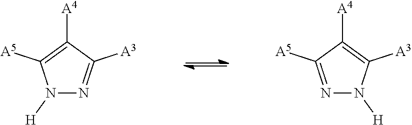

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

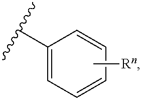

which is understood to be equivalent to a formula:

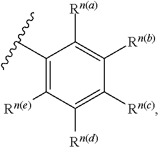

n is typically an integer. That is, R″ is understood to represent five independent substituents, R″^{(a)}, R″^{(b)}, R″^{(c)}, R″^{(d)}, and R″^{(e)}. By “independent substituents,” it is meant that each R substituent can be independently defined. For example, if in one instance R″^{(a)} is halogen, then R″^{(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### REFERENCES

All publications, patents, and patent applications mentioned in this specification are herein incorporated by reference to the same extent as if each individual publication, patent, or patent application was specifically and individually indicated to be incorporated by reference, including the references set forth in the following list:
1. International Patent Application Publication No. WO 2017/049295 for “Methods and Compositions of Inhibiting DCN1-UBC12 Interaction.”
2. Hammill, J. T.; Scott, D. C.; Min, J.; Connelly, M. C.; Holbrook, G.; Zhu, F.; Matheny, A.; Yang, L.; Singh, B.; Schulman, B. A.; et al. Piperidinyl Ureas Chemically Control Defective in Cullin Neddylation 1 (DCN1)-Mediated Cullin Neddylation. J Med Chem 2018, 61 (7), 2680-2693. DOI: 10.1021/acs.jmedchem.7b01277.
3. Hammill, J. T.; Bhasin, D.; Scott, D. C.; Min, J.; Chen, Y.; Lu, Y.; Yang, L.; Kim, H. S.; Connelly, M. C.; Hammill, C.; et al. Discovery of an Orally Bioavailable Inhibitor of Defective in Cullin Neddylation 1 (DCN1)-Mediated Cullin Neddylation. J Med Chem 2018, 61 (7), 2694-2706. DOI: 10.1021/acs.jmedchem.7b01282.

It will be understood that various details of the presently disclosed subject matter can be changed without departing from the scope of the subject matter disclosed herein.

Furthermore, the foregoing description is for the purpose of illustration only, and not for the purpose of limitation.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows structures of piperidinyl urea-based inhibitors of DCN1-UBE2M interaction previously reported by the present inventors.

FIG. 2 summarizes the modifications to the structures of FIG. 1 described herein.

FIG. 3 shows discrete regions of the structure of FIG. 1 considered for modification to achieve goals of lowering hERG binding activity and improving or maintaining biochemical and cellular potency and PK profiles.

FIG. 4 illustrates an embodiment of a method of making a modified compound according to the present disclosure.

FIG. 5 illustrates an alternative embodiment of a method of making a modified compound according to the present disclosure.

FIG. 6 illustrates compounds provided by optimization of the Hinge Pocket.

FIG. 7 illustrates compounds provided by optimization of the Hinge Pocket.

FIG. 8 illustrates compounds provided by optimization of the Hinge and Leu Pockets.

FIG. 9 illustrates compounds provided by optimization of the Ile Pocket.

FIG. 10 illustrates compounds provided by refining of the Hinge Pocket.

FIG. 11 illustrates compounds provided by optimization of the Leu Pocket.

FIG. 12 illustrates compounds provided by optimization of the Leu Pocket.

FIG. 13 illustrates compounds provided by optimization of the Ile Pocket.

FIG. 14 illustrates compounds provided by optimization of the Ile Pocket.

FIG. 15 summarizes modifications made to NAcM-OPT to provide a carbon bridge in the Leu Pocket.

FIG. 16 illustrates the modifications of FIG. 15.

FIG. 17 illustrates synthetic schema for providing bridged and unbridged isoquinoline carboxamide derivative compounds according to FIG. 15.

FIG. 18 illustrates synthetic schema for providing bridged and unbridged NAcM-OPT derivative compounds according to FIG. 15.

FIG. 19 shows representative compound formulae provided according to the schema of FIGS. 17-18.

FIG. 20 shows binding affinity of bridged isoquinoline analogues according to FIG. 19.

FIG. 21 shows binding affinity of bridged NAcM-OPT analogues according to FIG. 20.

FIG. 22 shows X-ray co-crystal structural modeling showing: A) compound 2 (NAcM-OPT) bound to different pockets of DCN1. B) compound 63 (JYH-019-089)'s benzyl group flip position to the N-Acetyl pocket.

FIG. 23 shows off-target hERG binding affinity of selected compounds.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

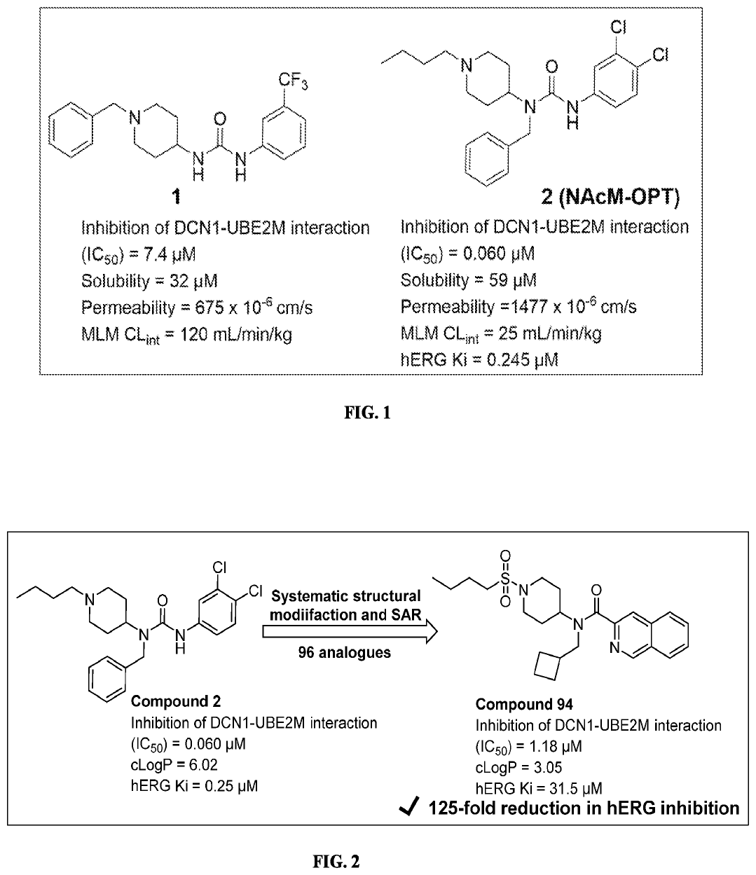

The present inventors have previously reported work relating to orally bioavailable small molecules, specifically piperidinyl urea-based molecules, for inhibiting DCN1-UBC12 interaction. See, for example, U.S. Published Patent Appl. No. 2022/0280488 and International Patent Appl. No. WO 2017/049295, see also FIG. 1. In the present work, consideration was given to potential modifications to/co-structures for such molecules with the goal of lowering hERG binding activity, and improving or maintaining biochemical and cellular potency and PK profiles. The structural modifications considered are summarized in FIG. 2.

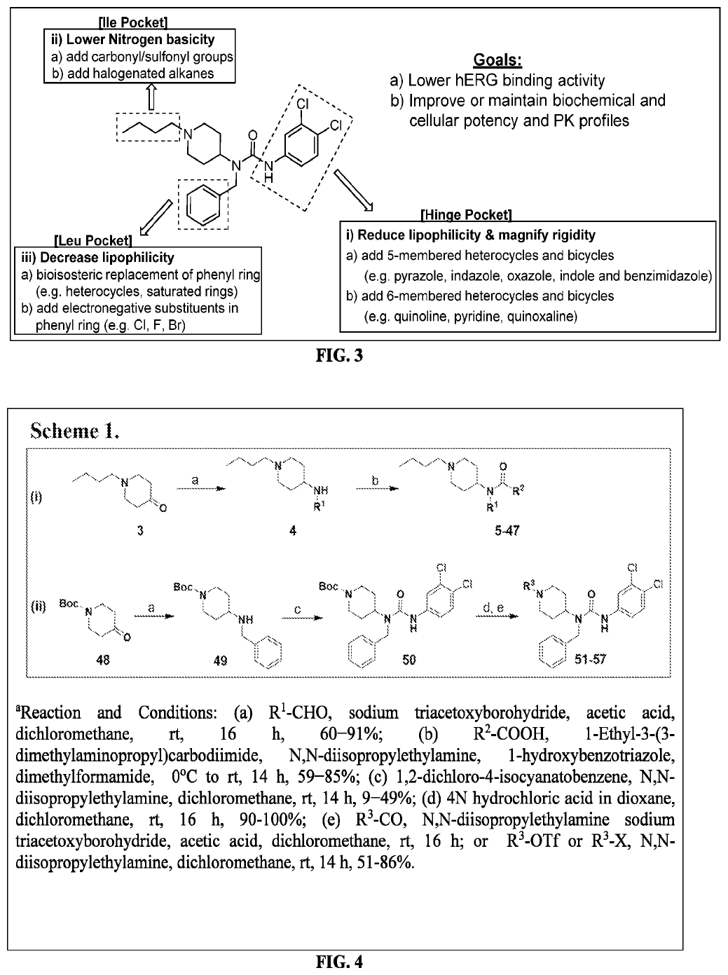

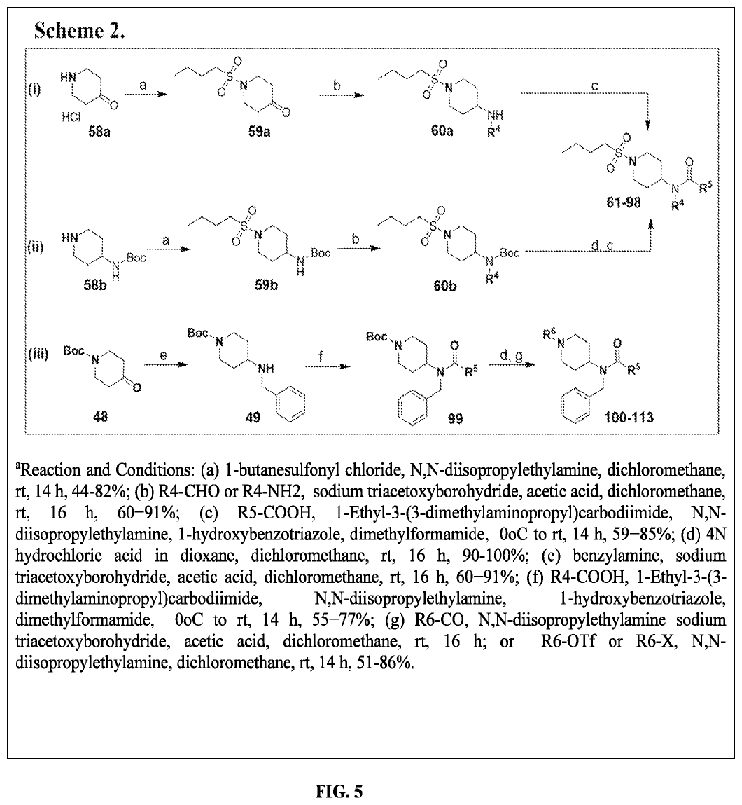

Three discrete regions or “pockets” of the molecule (NAcM-OPT, see FIG. 1) were considered for modification, designated the Ile Pocket, the Leu Pocket, and the Hinge Pocket (see FIG. 3). As described, the goal for the Hinge Pocket was to reduce lipophilicity and magnify rigidity of the molecule. The goal for the Ile Pocket was to lower nitrogen basicity. The goal for the Leu Pocket was to reduce lipophilicity. Substitutions considered for accomplishing these goals are as shown in the Figure. FIGS. 4 and 5 summarize methods, reagents, and reaction conditions used in producing various modified candidates for the present study.

### Procedure for TR-FRET Assay to Determine the Inhibition of DCN1-UBE2M Interaction:

Efficacy of the compound modifications evaluated was determined by determining half-maximal inhibitory concentration (IC₅₀) of molecules modified as shown by time-resolved fluorescence energy transfer (TR-FRET). TR-FRET assays were carried out in black 384-well microtiter plates at a final volume of 20 μL per well. To screen library compounds, the assay cocktail was prepared as a mixture of 50 nM biotin-DCN1, 20 nM AcUBE2M12-Alexa Fluor 488, and 2.5 nM Tb-streptavidin (Thermo Fisher) in assay buffer (25 mM HEPES, 100 mM NaCl, 0.1% Triton X-100, and 0.5 mM DTT, pH 7.5). The assay cocktail was incubated for 1 h at room temperature and distributed with a WellMate instrument (Matrix). Compounds to be screened were added to assay plates from DMSO stock solutions by pin transfer with 50SS pins (V&P Scientific). The assay mixture was incubated for 1 h at room temperature before measurement of the TR-FRET signal with a PHERAstar FS plate reader (BMG Labtech) equipped with modules for excitation at 337 nm and emission at 490 and 520 nm. The integration start was set to 100 ρs, and the integration time was set to 200 ρs. The number of flashes was fixed at 100. The 520/490 ratio was used as the TR-FRET signal in calculations. Assay endpoints were normalized from 0% (DMSO only) to 100% inhibition (unlabeled competitor peptide) for hit selection and curve fitting.

Procedure for hERG Binding Assay:

Filters were saturated overnight in 0.25% PEI in refrigerator then tubes were removed from −80° C. freezer (each tube contains 2.3 mL membrane suspension) for each intended experimental n; if obtaining multiple n for a single compound, ensure that cells are derived from different batches. Triturate volume in each tube prior to adding to the samples. Then protein assay was conducted to determine hERG channel protein concentration, subsequently prepared 50 nM [³H]dofetilide solution (5 nM final concentration). Check the Specific Activity and adjust the volume accordingly (generally 1 mL assay buffer required per filter). Drug solutions were prepared in H₂O and perform serial dilutions in the assay buffer; prepare 10 mM amitryptiline (final concentration 1 mM) in H₂O as nonspecific inhibitor. The concentrations range was between 0.1 uM-1 mM. The protein concentration was calculated in order to have a minimum of cell suspension necessary to obtain final protein concentration of 4 g/tube.

Samples tubes consisting of assay buffer, [³H]dofetilide, drug and protein were vortexed lightly and incubate on a shaker at room temperature for 1 hr. Then, filtered using Brandel, place filters in vials and add 5 mL scintillation cocktail and shaked vials for 30 minutes and placed in scintillation counter to get the results.

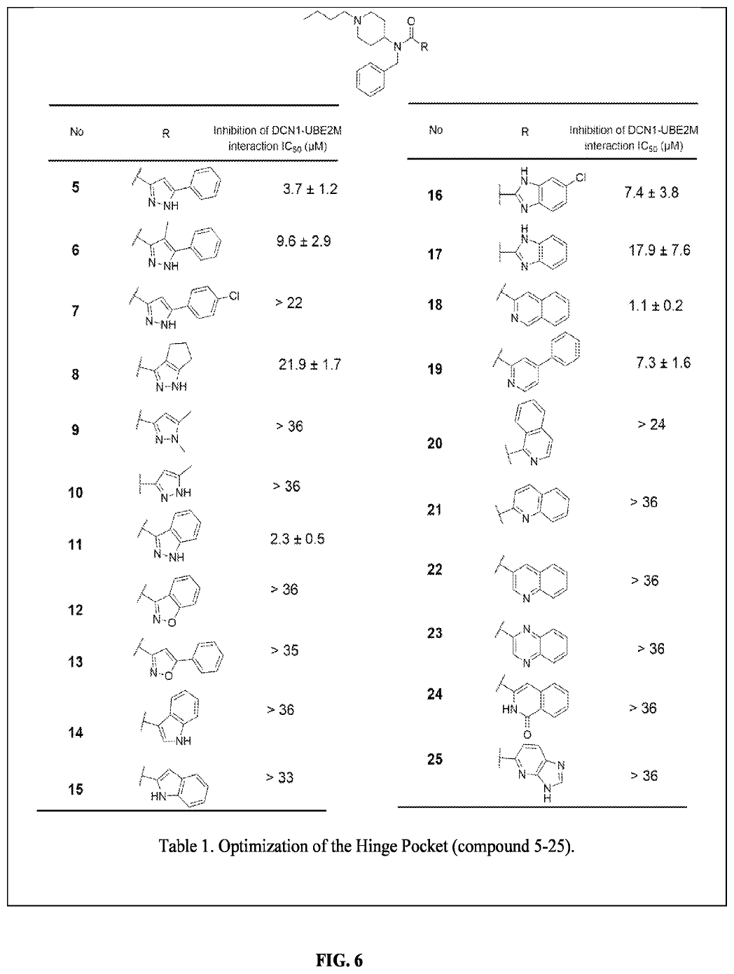

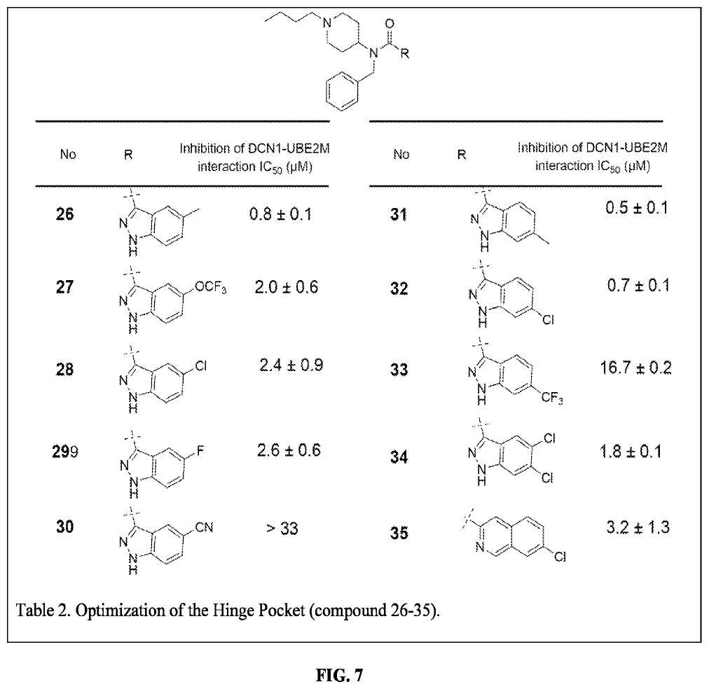

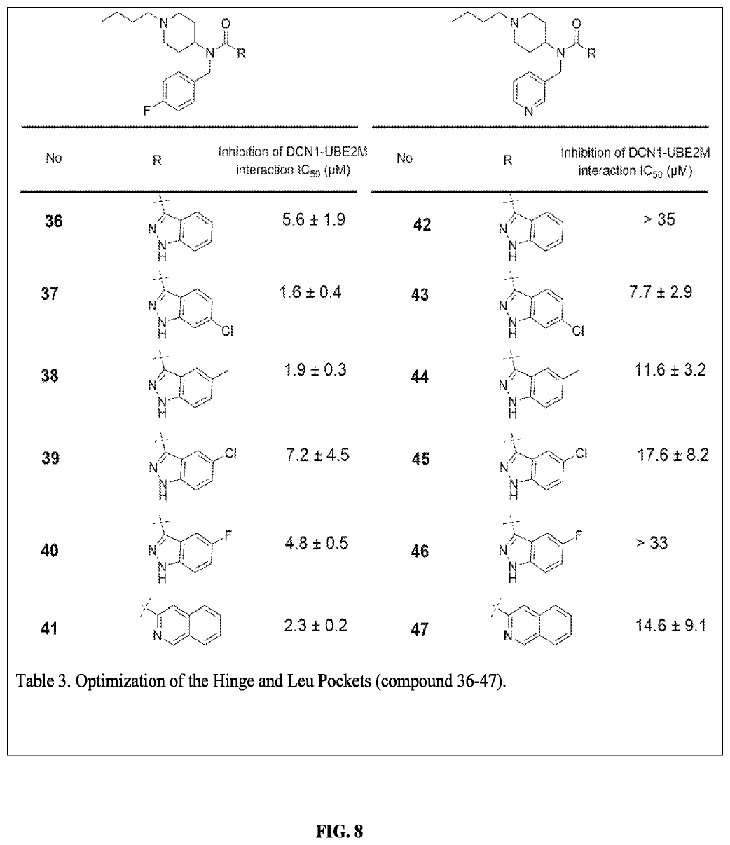

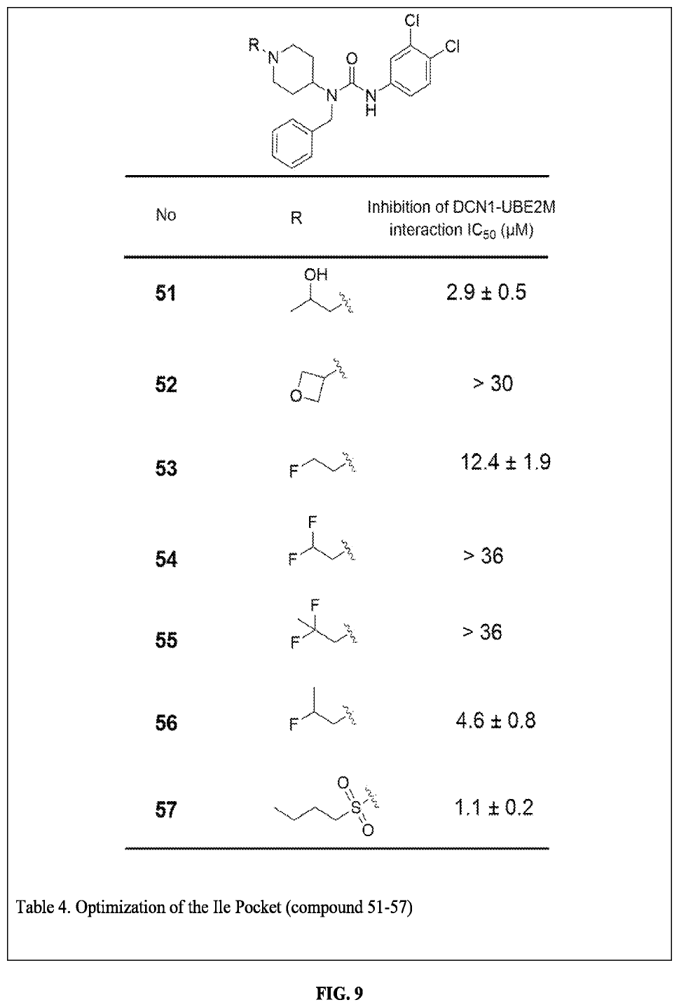

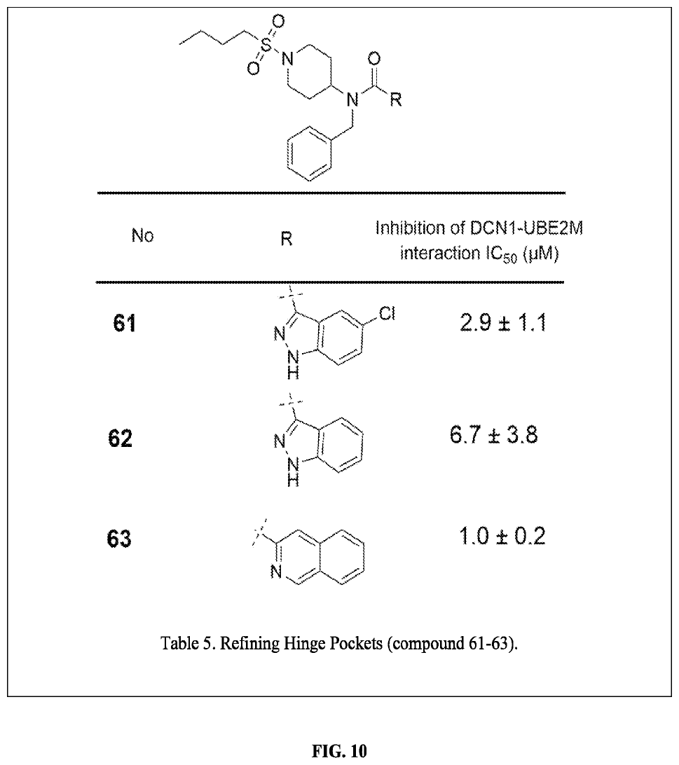

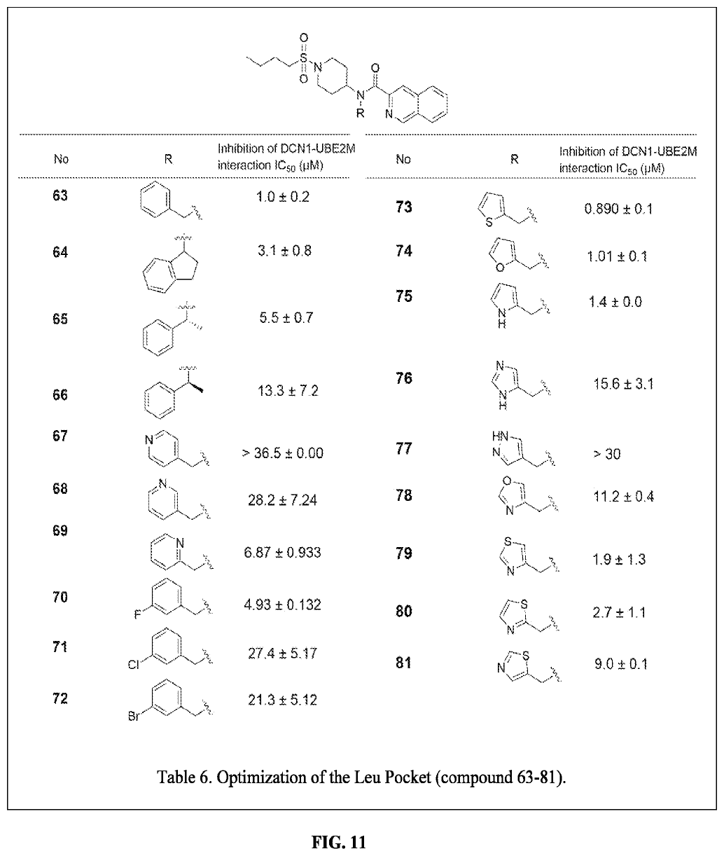

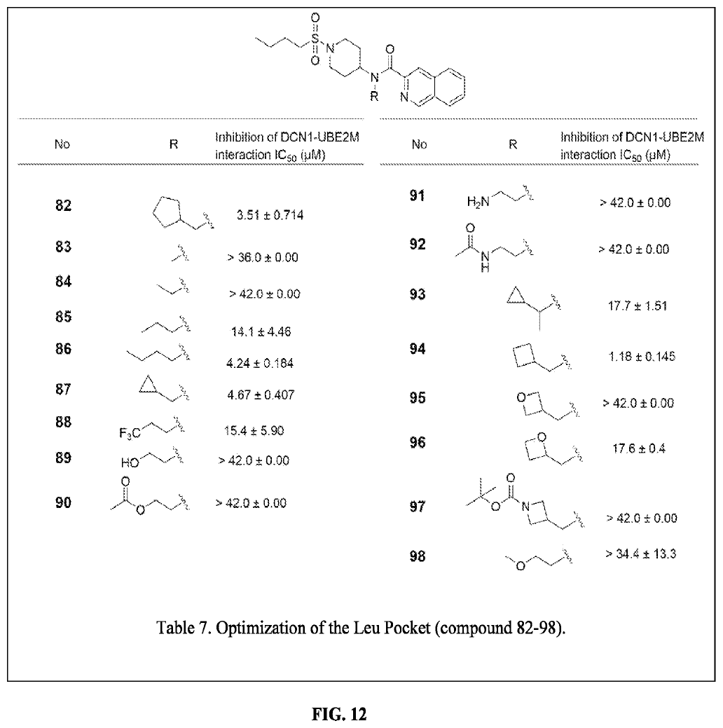

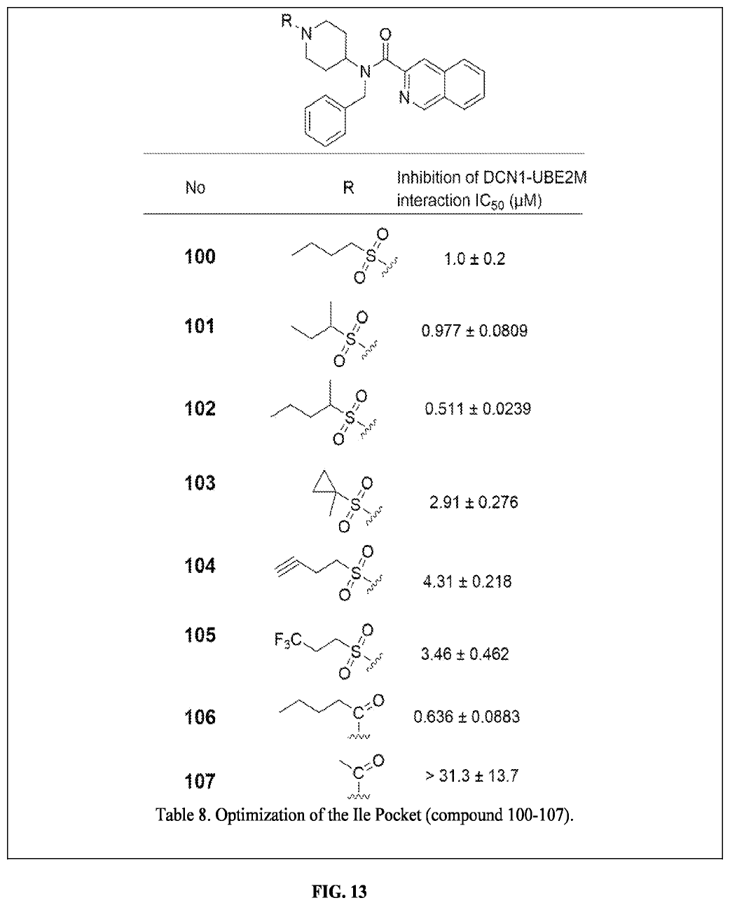

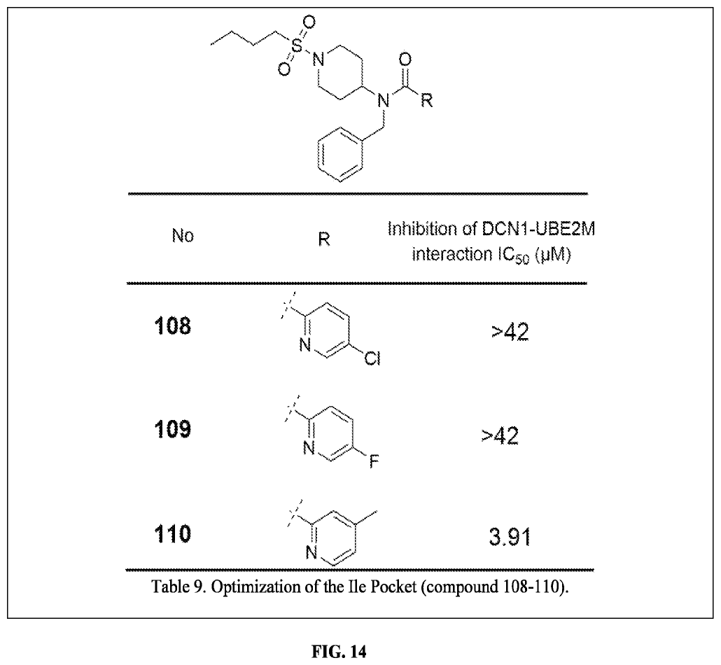

Results are shown in FIGS. 6-14. FIGS. 6-7 illustrate compound formulas provided by modification of the Hinge Pocket. FIG. 8 shows compound formulas provided by modification of the Hinge and Leu Pockets. FIG. 9 shows compound formulas provided by modification of the Ile Pocket. FIG. 10 shows additional compound formulas provided by modification of the Hinge Pocket. FIGS. 11-12 show compound formulas provided by optimization of the Leu Pocket. FIGS. 13-14 show compound formulas provided by optimization of the Ile Pocket.

Specific chemical characterizations of particular compounds according to the foregoing description will now be shown.

### Synthesis of N-benzyl-N-(1-butylpiperidin-4-yl)-5-phenyl-1H-pyrazole-3-carboxamide (5)

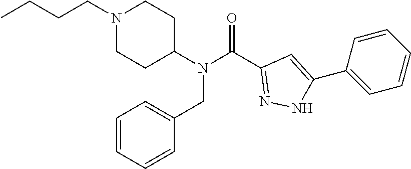 [SMILES: CCCCN1CCC(N(Cc2ccccc2)C(=O)c2cc(-c3ccccc3)[nH]n2)CC1]

Yield 83%, Ivory gel, ¹H NMR (500 MHz, Chloroform-d) δ 9.25 (s, 0.6H), 9.06 (s, 0.4H), 8.65-8.41 (m, 2H), 8.20-7.63 (m, 5H), 7.32-7.20 (m, 2H), 4.88 (s, 1H), 4.77-4.75 (1.4H), 4.29-4.20 (m, 0.6H), 3.85 (dd, J=49.8, 12.3 Hz, 2H), 2.92-2.79 (m, 3H), 2.57 (t, 0.1=12.3 Hz, 1H), 2.05-1.65 (m, 6H), 1.47-1.36 (m, 2H), 0.93 (dt, J=27.4, 7.4 Hz, 3H).

LRMS (ESI+) m/z calcd for C₂₅H₃₁N₄O₃S⁺ [M+H]⁺ 417.2. found 417.1

### Synthesis of N-benzyl-N-(1-butylpiperidin-4-yl)-4-methyl-5-phenyl-1H-pyrazole-3-carboxamide (6)

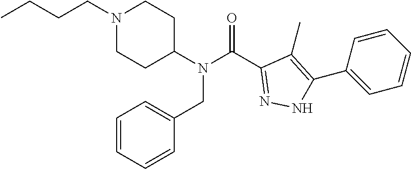 [SMILES: CCCCN1CCC(N(Cc2ccccc2)C(=O)c2n[nH]c(-c3ccccc3)c2C)CC1]

Yield 62%, Pale yellow gel, ¹H NMR (500 MHz, Chloroform-d) δ 9.25 (s, 0.6H), 9.06 (s, 0.4H), 8.65-8.41 (m, 2H), 8.20-7.63 (m, 5H), 7.32-7.20 (m, 2H), 4.88 (s, 1H), 4.77-4.75 (1.4H), 4.29-4.20 (m, 0.6H), 3.85 (dd, J=49.8, 12.3 Hz, 2H), 2.92-2.79 (m, 3H), 2.57 (t, J=12.3 Hz, 1H), 2.05-1.65 (m, 6H), 1.47-1.36 (m, 2H), 0.93 (dt, J=27.4, 7.4 Hz, 3H).

LRMS (ESI+) m/z calcd for C₂₅H₃₁N₄O₃S⁺ [M+H]⁺ 431.2. found 431.1

### Synthesis of N-benzyl-N-(1-butylpiperidin-4-yl)-5-(4-chlorophenyl)-1H-pyrazole-3-carboxamide (7)

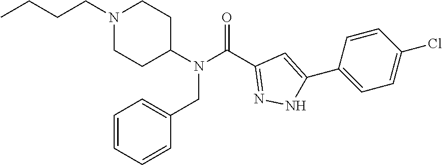 [SMILES: CCCCN1CCC(N(Cc2ccccc2)C(=O)c2cc(-c3ccc(Cl)cc3)[nH]n2)CC1]

Yield 69%, Ivory amorphous powder, ¹H NMR (500 MHz, Chloroform-d) δ 9.25 (s, 0.6H), 9.06 (s, 0.4H), 8.65-8.41 (m, 2H), 8.20-7.63 (m, 5H), 7.32-7.20 (m, 2H), 4.88 (s, 1H), 4.77-4.75 (1.4H), 4.29-4.20 (m, 0.6H), 3.85 (dd, J=49.8, 12.3 Hz, 2H), 2.92-2.79 (m, 3H), 2.57 (t, J=12.3 Hz, 1H), 2.05-1.65 (m, 6H), 1.47-1.36 (m, 2H), 0.93 (dt, J=27.4, 7.4 Hz, 3H).

LRMS (ESI+) m/z calcd for C₂₅H₃₁N₄O₃S⁺ [M+H]⁺ 451.1. found 451.03

### Synthesis of N-benzyl-N-(1-butylpiperidin-4-yl)-1,4,5,6-tetrahydrocyclopenta[c]pyrazole-3-carboxamide (8)

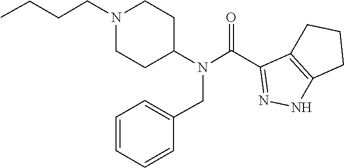 [SMILES: CCCCN1CCC(N(Cc2ccccc2)C(=O)c2n[nH]c3c2CCC3)CC1]

### Synthesis of N-benzyl-N-(1-butylpiperidin-4-yl)-1,5-dimethyl-1H-pyrazole-3-carboxamide (9)

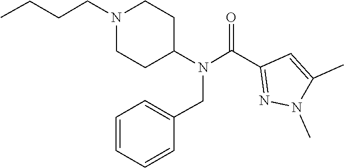 [SMILES: CCCCN1CCC(N(Cc2ccccc2)C(=O)c2cc(C)n(C)n2)CC1]

Yield 54%, Colorless gel, ¹H NMR (500 MHz, Chloroform-d) δ 7.31-7.24 (m, 3H), 7.24-7.14 (m, 2H), 6.45-6.34 (m, 1H), 5.04 (s, 1H), 4.73 (s, 1H), 4.70-4.60 (brs, 1H), 3.81-3.67 (m, 3H), 2.94 (brs, 2H), 2.37 (s, 3H), 2.37-2.19 (m, 2H), 2.04 (m, 1H), 1.93-1.63 (m, 5H), 1.43 (m, 2H), 1.33-1.23 (m, 2H), 0.88 (t, J=7.3 Hz, 3H).

LRMS (ESI+) m/z calcd for C₂₅H₃₁N₄O₃S⁺ [M+H]⁺ 369.5. found 369.4

### Synthesis of N-benzyl-N-(1-butylpiperidin-4-yl)-5-methyl-1H-pyrazole-3-carboxamide (10)

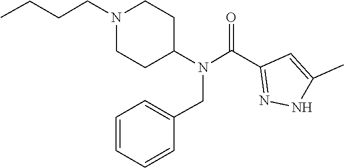 [SMILES: CCCCN1CCC(N(Cc2ccccc2)C(=O)c2cc(C)[nH]n2)CC1]

### Synthesis of N-benzyl-N-(1-butylpiperidin-4-yl)-1H-indazole-3-carboxamide (11)

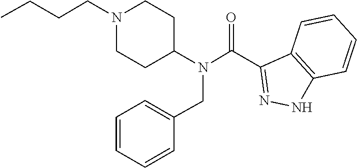 [SMILES: CCCCN1CCC(N(Cc2ccccc2)C(=O)c2n[nH]c3ccccc23)CC1]

Yield 54%, Colorless gel, ¹H NMR (500 MHz, Chloroform-d) δ 10.75 (s, 1H), 8.20 (d, 7.4 Hz, 1H), 7.55-7.31 (m, 3H), 7.31-7.23 (m, 3H), 7.22-7.08 (m, 2H), 5.18 (s, 1H), 4.84 (s, 1H), 4.75 (s, 1H), 3.02 (m, 2H), 2.55-2.09 (m, 3H), 1.88 (m, 2H), 1.63-1.35 (m, 2H), 1.35-1.12 (m, 3H), 0.95-0.79 (m, 3H).

### Synthesis of N-benzyl-N-(1-butylpiperidin-4-yl)benzo[d]isoxazole-3-carboxamide (12)

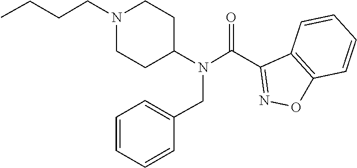 [SMILES: CCCCN1CCC(N(Cc2ccccc2)C(=O)c2noc3ccccc23)CC1]

Yield 6%, Colorless gel, H NMR (500 MHz, Chloroform-d) δ 7.99 (d, J=7.9 Hz, 0.5H), 7.89 (d, J=8.0 Hz, 0.5H), 7.68-7.60 (m, 1.3H), 7.56-7.53 (m, 0.7H), 7.43-7.39 (m, 0.7H), 7.38-7.30 (m, 2.5H), 7.26-7.22 (m, 0.5H), 7.22-7.16 (m, 1.5H), 7.15-7.09 (m, 0.5H), 4.95 (s, 1H), 4.88 (s, 1H), 4.72 (s, 0.5H), 4.34 (s, 0.5H), 3.11-2.90 (m, 2H), 2.44-2.23 (m, 2H), 2.00-1.74 (m, 5H), 1.56-1.40 (m, 3H), 1.35-1.27 (m, 2H), 0.92-0.85 (m, 3H).

### Synthesis of N-benzyl-N-(1-butylpiperidin-4-yl)-5-phenylisoxazole-3-carboxamide (13)

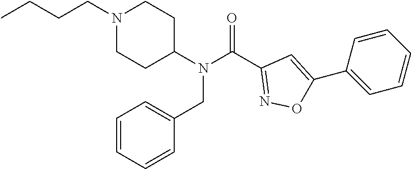 [SMILES: CCCCN1CCC(N(Cc2ccccc2)C(=O)c2cc(-c3ccccc3)on2)CC1]

Yield 20%, Colorless gel, ¹H NMR (500 MHz, Chloroform-d) δ 7.86-7.79 (m, 1H), 7.74-7.68 (m, 1H), 7.52-7.48 (m, 1.5H), 7.48-7.41 (m, 1.5H), 7.35-7.26 (m, 3H), 7.24-7.18 (m, 2H), 6.90 (s, 0.5H), 6.72 (s, 0.5H), 4.95 (s, 1H), 4.81 (s, 1H), 4.66 (s, 0.5H), 4.46 (s, 0.5H), 3.17-2.93 (m, 2H), 2.50-2.28 (m, 2H), 2.20-1.91 (m, 3H), 1.83-1.73 (m, 2H), 1.56-1.41 (m, 3H), 1.36-1.26 (m, 2H), 0.93-0.85 (m, 3H).

### Synthesis of N-benzyl-N-(1-butylpiperidin-4-yl)-1H-indole-3-carboxamide (14

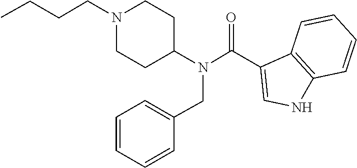 [SMILES: CCCCN1CCC(N(Cc2ccccc2)C(=O)c2c[nH]c3ccccc23)CC1]

Yield 54%, Colorless gel, ¹H NMR (500 MHz, Chloroform-d) δ 8.98 (s, 1H), 7.89 (s, 1H), 7.35-7.23 (m, 6H), 7.23-7.18 (m, 2H), 7.12 (d, J=2.6 Hz, 1H), 4.75 (s, 2H), 4.59-4.39 (m, 1H), 2.99 (s, 2H), 2.34 (s, 2H), 2.05-1.83 (m, 4H), 1.74 (d, J=9.1 Hz, 2H), 1.52-1.40 (m, 2H), 1.32-1.23 (m, 2H), 0.88 (t, J=7.3 Hz, 3H).

### Synthesis of N-benzyl-N-(1-butylpiperidin-4-yl)-1H-indole-2-carboxamide (15)

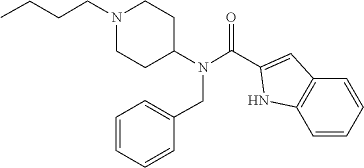 [SMILES: CCCCN1CCC(N(Cc2ccccc2)C(=O)c2cc3ccccc3[nH]2)CC1]

Yield 54%, Colorless gel, ¹H NMR (500 MHz, Chloroform-d) δ 9.39 (s, 1H), 7.65-7.44 (m, 1H), 7.40 (t, J=6.9 Hz, 1H), 7.39-7.31 (m, 3H), 7.31-7.24 (m, 3H), 7.13-7.03 (m, 1H), 6.71 (brs, 1H), 4.94 (brs, 2H), 4.67 (d, J=40.1 Hz, 1H), 2.99 (d, J=8.5 Hz, 2H), 2.37-2.27 (m, 2H), 2.06 (brs, 2H), 1.89-1.69 (m, 4H), 1.50-1.40 (m, 2H), 1.30 (q, J=7.3 Hz, 2H), 0.90 (t, J=7.3 Hz, 3H).

LRMS (ESI+) m/z calcd for C₂₅H₃₁N₄O₃S⁺ [M+H]⁺ 390.5. found 390.1

### Synthesis of N-benzyl-N-(1-butylpiperidin-4-yl)-6-chloro-1H-benzo[d]imidazole-2-carboxamide (16)

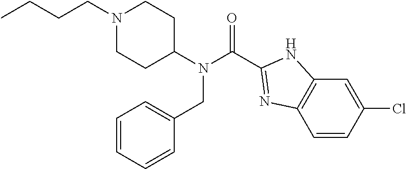 [SMILES: CCCCN1CCC(N(Cc2ccccc2)C(=O)c2nc3ccc(Cl)cc3[nH]2)CC1]

Yield 52%, Yellow solid, ¹H NMR (500 MHz, Chloroform-d) δ 8.98 (s, 1H), 7.90 (s, 1H), 7.31-7.23 (m, 6H), 7.23-7.18 (m, 2H), 7.12 (d, J=2.6 Hz, 1H), 4.75 (s, 2H), 4.59-4.39 (m, 1H), 2.99 (s, 2H), 2.34 (s, 2H), 2.05-1.83 (m, 4H), 1.74 (d, J=9.1 Hz, 2H), 1.55-1.40 (m, 2H), 1.33-1.23 (m, 2H), 0.86 (t, J=7.3 Hz, 3H).

LRMS (ESI+) m/z calcd for C₂₅H₃₁N₄O₃S⁺ [M+H]⁺ 425.2. found 424.9

### Synthesis of N-benzyl-N-(1-butylpiperidin-4-yl)-1H-benzo[d]imidazole-2-carboxamide (17)

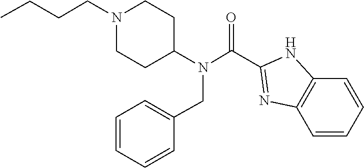 [SMILES: CCCCN1CCC(N(Cc2ccccc2)C(=O)c2nc3ccccc3[nH]2)CC1]

Yield 61%, Ivory solid, ¹H NMR (500 MHz, Chloroform-d) δ 8.98 (s, 1H), 7.89 (s, 1H), 7.35-7.23 (m, 6H), 7.23-7.18 (m, 2H), 7.12 (d, J=2.6 Hz, 1H), 4.75 (s, 2H), 4.59-4.39 (m, 1H), 2.99 (s, 2H), 2.34 (s, 2H), 2.05-1.83 (m, 4H), 1.74 (d, J=9.1 Hz, 2H), 1.52-1.40 (m, 2H), 1.32-1.23 (m, 2H), 0.88 (t, J=7.3 Hz, 3H).

LRMS (ESI+) m/z calcd for C₂₅H₃₁N₄O₃S⁺ [M+H]⁺ 391.2. found 391.1

### Synthesis of N-benzyl-N-(1-butylpiperidin-4-yl)isoquinoline-3-carboxamide (18)

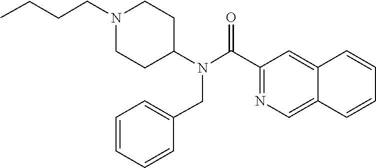 [SMILES: CCCCN1CCC(N(Cc2ccccc2)C(=O)c2cc3ccccc3cn2)CC1]

Yield 78%, Pale yellow gel, ¹H NMR (500 MHz, Chloroform-d) δ 8.99 (s, 1H), 7.46 (s, 1H), 7.35-7.23 (m, 6H), 7.23-7.18 (m, 2H), 7.12 (d, J=2.6 Hz, 1H), 4.75 (s, 2H), 4.59-4.39 (m, 1H), 2.99 (s, 2H), 2.34 (s, 2H), 2.05-1.83 (m, 4H), 1.74 (d, J=9.1 Hz, 2H), 1.52-1.40 (m, 2H), 1.32-1.23 (m, 2H), 0.86 (t, J=7.3 Hz, 3H).

LRMS (ESI+) m/z calcd for C₂₅H₃₁N₄O₃S⁺ [M+H]⁺ 402.2. found 402.1

### Synthesis of N-benzyl-N-(1-butylpiperidin-4-yl)-4-phenylpicolinamide (19)

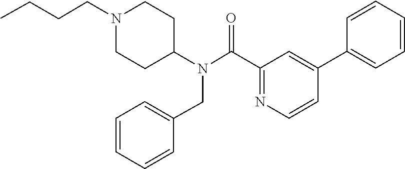 [SMILES: CCCCN1CCC(N(Cc2ccccc2)C(=O)c2cc(-c3ccccc3)ccn2)CC1]

Yield 79%, Ivory sticky gel, ¹H NMR (500 MHz, Chloroform-d) δ 8.65 (d, J=5.1 Hz, 0.5H), 8.52 (d, J=5.1 Hz, 0.5H), 7.88 (s, 1H), 7.70 (d, J=7.2 Hz, 1H), 7.62-7.56 (m, 1H), 7.54-7.36 (m, 5H), 7.32 (t, J=7.6 Hz, 1H), 7.24-7.11 (m, 3H), 4.85-4.75 (m, 2H), 3.90 (s, 1H), 3.17-2.85 (m, 2H), 2.55-2.16 (m, 3H), 1.93-1.74 (m, 3H), 1.59 (s, 2H), 1.47-1.20 (m, 4H), 0.98-0.81 (m, 3H).

LRMS (ESI+) m/z calcd for C₂₅H₃₁N₄O₃S⁺ [M+H]⁺ 428.2. found 428.1

### Synthesis of N-benzyl-N-(1-butylpiperidin-4-yl)isoquinoline-1-carboxamide (20)

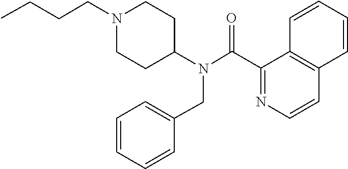 [SMILES: CCCCN1CCC(N(Cc2ccccc2)C(=O)c2nccc3ccccc23)CC1]

Yield 76%, Ivory sticky gel, ¹H NMR (500 MHz, Chloroform-d) δ 8.54 (d, J=5.7 Hz, 0.5H), 8.44 (d, J=5.7 Hz, 0.5H), 8.08 (d, J=8.4 Hz, 0.5H), 7.93 (d, J=8.3 Hz, 0.5H), 7.89 (d, J=8.2 Hz, 0.5H), 7.76 7.70 (m, 1.5H), 7.66-7.61 (m, 1H), 7.59-7.50 (m, 2H), 7.38 (t, J=7.6 Hz, 1H), 7.29-7.26 (m, 1H), 7.14-6.97 (m, 2H), 4.92 (s, 1H), 4.84 (s, 0.5H), 4.48 (s, 1H), 3.33 (s, 0.5H), 3.15 (s, 1H), 2.81 (s, 1H), 2.51-2.09 (m, 3H), 1.96-1.79 (m, 2H), 1.71-1.60 (m, 3H), 1.38-1.29 (m, 2H), 1.28-1.16 (m, 2H), 0.92 (t, J=7.3 Hz, 1.5H), 0.83 (t, J=7.3 Hz, 1.5H).

LRMS (ESI+) m/z calcd for C₂₅H₃₁N₄O₃S⁺ [M+H]⁺ 402.2. found 402.1

### Synthesis of N-benzyl-N-(1-butylpiperidin-4-yl)quinoline-2-carboxamide (21)

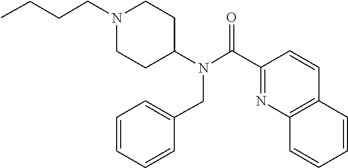 [SMILES: CCCCN1CCC(N(Cc2ccccc2)C(=O)c2ccc3ccccc3n2)CC1]

Yield 82%, Ivory sticky gel, ¹H NMR (500 MHz, Chloroform-d) δ 8.29 (d, 0.1=8.4 Hz, 0.5H), 8.10 (d, J=8.4 Hz, 1H), 7.95 (d, J=8.5 Hz, 0.5H), 7.88 (d, J=8.1 Hz, 0.5H), 7.80-7.75 (m, 1.5H), 7.71-7.66 (m, 0.5H), 7.64-7.60 (m, 0.5H), 7.59-7.51 (m, 1H), 7.41 (d, J=7.5 Hz, 1H), 7.33 (t, J=7.6 Hz, 1H), 7.26-7.21 (m, 1H), 7.20-7.10 (m, 2H), 4.85 (s, 2H), 4.71 (s, 0.5H), 4.03 (s, 0.5H), 3.07 (s, 1H), 2.95-2.85 (m, 1H), 2.40 (s, 1H), 2.18 (s, 2H), 1.89 (s, 3H), 1.56 (d, J=39.7 Hz, 2H), 1.43-1.21 (m, 4H), 0.91 (dd, J=15.3, 7.9 Hz, 1H), 0.85 (t, J=7.3 Hz, 1H).

### Synthesis of N-benzyl-N-(1-butylpiperidin-4-yl)quinoline-3-carboxamide (22)

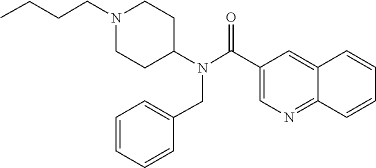 [SMILES: CCCCN1CCC(N(Cc2ccccc2)C(=O)c2cnc3ccccc3c2)CC1]

Yield 37%, Ivory sticky gel, ¹H NMR (500 MHz, Chloroform-d) δ 9.06-8.86 (m, 1H), 8.35-8.04 (m, 2H), 7.94-7.50 (m, 3H), 7.40-7.13 (m, 5H), 4.90-4.53 (m, 3H), 3.70 (s, 1H), 3.23-2.78 (m, 2H), 2.48-2.09 (m, 3H), 1.92 (s, 2H), 1.60 (s, 2H), 1.43-1.14 (m, 4H), 0.97-0.78 (m, 3H).

LRMS (ESI+) m/z calcd for C₂₅H₃₁N₄O₃S⁺ [M+H]⁺ 402.2. found 402.1

### Synthesis of N-benzyl-N-(1-butylpiperidin-4-yl)quinoxaline-2-carboxamide (23)

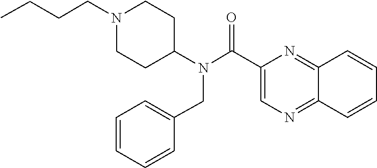 [SMILES: CCCCN1CCC(N(Cc2ccccc2)C(=O)c2cnc3ccccc3n2)CC1]

Yield 75%, Ivory sticky gel, ¹H NMR (500 MHz, Chloroform-d) δ 9.23 (s, 0.5H), 8.96 (s, 0.5H), 8.18 (dd, J=7.2, 2.4 Hz, 1H), 8.11-8.01 (m, 1H), 7.95-7.90 (m, 1H), 7.90-7.81 (m, 1H), 7.81-7.73 (m, 1H), 7.40 (d, J=7.5 Hz, 1H), 7.34 (t, J=7.6 Hz, 1H), 7.26-7.23 (m, 1H), 7.17-7.10 (m, 2H), 4.87 (d, J=7.4 Hz, 2H), 4.75 (s, 1H), 4.07 (s, 1H), 3.18-3.05 (m, 1H), 2.99-2.87 (m, 1H), 2.49-2.35 (m, 1H), 2.29-2.13 (m, 2H), 2.03-1.85 (m, 3H), 1.67 (d, J=77.2 Hz, 2H), 1.46-1.17 (m, 4H), 0.92 (t, J=7.3 Hz, 1.5H), 0.87 (t, J=7.3 Hz, 1.5H).

LRMS (ESI+) m/z calcd for C₂₅H₃₁N₄O₃S⁺ [M+H]⁺ 403.2. found 403.1

### Synthesis of N-benzyl-N-(1-butylpiperidin-4-yl)-1-oxo-1,2-dihydroisoquinoline-3-carboxamide (24)

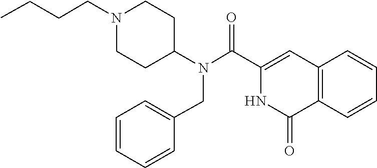 [SMILES: CCCCN1CCC(N(Cc2ccccc2)C(=O)c2cc3ccccc3c(=O)[nH]2)CC1]

Yield 54%, Ivory amorphous powder, ¹H NMR (500 MHz, Chloroform-d) δ 9.24 (s, 1H), 8.40 (d, J=8.0 Hz, 1H), 7.64 (t, J=7.4 Hz, 1H), 7.57-7.51 (m, 1H), 7.44-7.38 (m, 1H), 7.38-7.32 (m, 2H), 7.29-7.24 (m, 3H), 6.69 (s, 1H), 4.79 (s, 2H), 4.33 (s, 1H), 2.98 (d, J=9.7 Hz, 2H), 2.33-2.25 (m, 2H), 2.01-1.82 (m, 4H), 1.78 (d, J=10.2 Hz, 2H), 1.48-1.38 (m, 2H), 1.34-1.23 (m, 2H), 0.89 (t, J=7.3 Hz, 3H).

LRMS (ESI+) m/z calcd for C₂₅H₃₁N₄O₃S⁺ [M+H]⁺ 418.2. found 418.2

### Synthesis of N-benzyl-N-(1-butylpiperidin-4-yl)-3H-imidazo[4,5-b]pyridine-5-carboxamide (25)

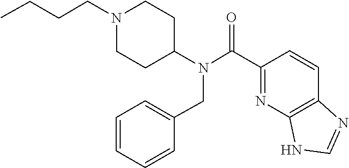 [SMILES: CCCCN1CCC(N(Cc2ccccc2)C(=O)c2ccc3nc[nH]c3n2)CC1]

Yield 39%, Pale ivory gel, ¹H NMR (500 MHz, Chloroform-d) δ 8.98 (s, 1H), 7.89 (s, 1H), 7.35-7.23 (m, 6H), 7.23-7.18 (m, 2H), 7.12 (d, J=2.6 Hz, 1H), 4.75 (s, 2H), 4.59-4.39 (m, 1H), 2.99 (s, 2H), 2.34 (s, 2H), 2.05-1.83 (m, 4H), 1.74 (d, J=9.1 Hz, 2H), 1.52-1.40 (m, 2H), 1.32-1.23 (m, 2H), 0.88 (t, J=7.3 Hz, 3H).

LRMS (ESI+) m/z calcd for C₂₅H₃₁N₄O₃S⁺ [M+H]⁺ 392.2. found 392.4

### Synthesis of N-benzyl-N-(1-butylpiperidin-4-yl)-5-methyl-1H-indazole-3-carboxamide (26)

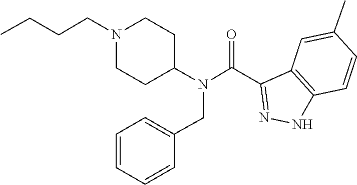 [SMILES: CCCCN1CCC(N(Cc2ccccc2)C(=O)c2n[nH]c3ccc(C)cc23)CC1]

Yield 10%, Ivory gel, ¹H NMR (500 MHz, Chloroform-d) δ 10.84 (s, 1H), 8.29 (s, 0.5H), 8.21 (s, 0.5H), 7.46-7.26 (m, 5H), 7.23-7.10 (m, 3H), 5.17 (s, 1H), 4.82 (s, 1H), 4.77 (brs, 1H), 3.00 (m, 2H), 2.45-2.24 (m, 2H), 2.16 (brs, 1H), 1.99-1.70 (m, 5H), 1.55-1.38 (m, 2H), 1.38-1.17 (m, 2H), 0.92-0.82 (m, 3H).

LRMS (ESI+) m/z calcd for C₂₅H₃₁N₄O₃S⁺ [M+H]⁺ 405.2. found 405.1

### Synthesis of N-benzyl-N-(1-butylpiperidin-4-yl)-5-(trifluoromethoxy)-1H-indazole-3-carboxamide (27)

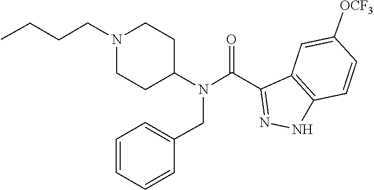 [SMILES: CCCCN1CCC(N(Cc2ccccc2)C(=O)c2n[nH]c3ccc(OC(F)(F)F)cc23)CC1]

Yield 12%, Pale yellow sticky gel, H NMR (500 MHz, Chloroform-d) δ 11.04 (brs, 1H), 8.20-8.04 (m, 1H), 7.61-7.45 (m, 1H), 7.36-7.10 (m, 6H), 5.29 (s, 1H), 4.84 (s, 2H), 3.32-3.02 (m, 2H), 2.68-1.94 (m, 5H), 1.94-1.72 (m, 2H), 1.72-1.42 (m, 3H), 1.32 (s, 2H), 0.95-0.83 (m, 3H).

LRMS (ESI+) m/z calcd for C₂₅H₃₁N₄O₃S⁺ [M+H]⁺ 475.2. found 475.1

### Synthesis of N-benzyl-N-(1-butylpiperidin-4-yl)-5-chloro-1H-indazole-3-carboxamide (28)

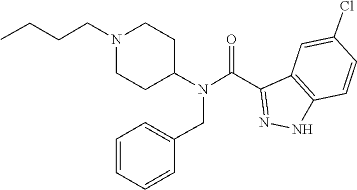 [SMILES: CCCCN1CCC(N(Cc2ccccc2)C(=O)c2n[nH]c3ccc(Cl)cc23)CC1]

Yield 13%, Yellow gel, ¹H NMR (500 MHz, Chloroform-d) δ 10.84 (s, 1H), 8.29 (s, 0.5H), 8.21 (s, 0.5H), 7.46-7.26 (m, 5H), 7.23-7.10 (m, 3H), 5.17 (s, 1H), 4.82 (s, 1H), 4.77 (brs, 1H), 3.00 (m, 2H), 2.45-2.24 (m, 2H), 2.16 (brs, 1H), 1.99-1.70 (m, 5H), 1.55-1.38 (m, 2H), 1.38-1.17 (m, 2H), 0.92-0.82 (m, 3H).

LRMS (ESI+) m/z calcd for C₂₅H₃₁N₄O₃S⁺ [M+H]⁺ 425.2. found 425.5

### Synthesis of N-benzyl-N-(1-butylpiperidin-4-yl)-5-fluoro-1H-indazole-3-carboxamide (29)

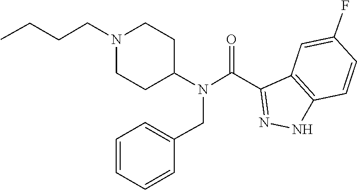 [SMILES: CCCCN1CCC(N(Cc2ccccc2)C(=O)c2n[nH]c3ccc(F)cc23)CC1]

Yield 11%, Yellow gel, ¹H NMR (500 MHz, Chloroform-d) δ 10.84 (s, 1H), 8.29 (s, 0.5H), 8.21 (s, 0.5H), 7.46-7.26 (m, 5H), 7.23-7.10 (m, 3H), 5.17 (s, 1H), 4.82 (s, 1H), 4.77 (brs, 1H), 3.00 (m, 2H), 2.45-2.24 (m, 2H), 2.16 (brs, 1H), 1.99-1.70 (m, 5H), 1.55-1.38 (m, 2H), 1.38-1.17 (m, 2H), 0.92-0.82 (m, 3H).

LRMS (ESI+) m/z calcd for C₂₅H₃₁N₄O₃S⁺ [M+H]⁺ 409.2. found 409.2

### Synthesis of N-benzyl-N-(1-butylpiperidin-4-yl)-5-cyano-1H-indazole-3-carboxamide (30)

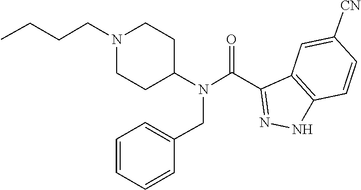 [SMILES: CCCCN1CCC(N(Cc2ccccc2)C(=O)c2n[nH]c3ccc(C#N)cc23)CC1]

Yield 14%, Pale yellow sticky gel, ¹H NMR (500 MHz, Chloroform-d) δ 11.37 (brs, 1H), 8.76-8.54 (m, 1H), 7.64-7.49 (m, 2H), 7.36-7.23 (m, 2H), 7.23-7.06 (m, 3H), 5.26 (s, 1H), 4.83 (brs, 2H), 3.31-2.97 (m, 2H), 2.66-2.30 (m, 3H), 2.17-1.71 (m, 4H), 1.67-1.43 (m, 3H), 1.38-1.25 (m, 2H), 0.97-0.81 (m, 3H).

### Synthesis of N-benzyl-N-(1-butylpiperidin-4-yl)-6-methyl-1H-indazole-3-carboxamide (31)

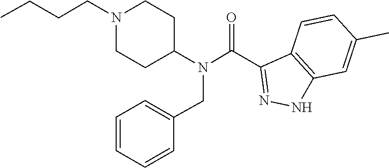 [SMILES: CCCCN1CCC(N(Cc2ccccc2)C(=O)c2n[nH]c3cc(C)ccc23)CC1]

Yield 11%, Pale yellow sticky gel, H NMR (500 MHz, Chloroform-d) δ 10.43 (s, 1H), 8.16-8.00 (m, 1H), 7.40-7.09 (m, 7H), 5.24 (s, 1H), 4.85 (s, 2H), 3.33-3.02 (m, 2H), 2.71-1.93 (m, 5H), 1.79 (s, 2H), 1.71-1.48 (m, 3H), 1.41-1.29 (m, 2H), 0.95-0.82 (m, 3H).

### Synthesis of N-benzyl-N-(1-butylpiperidin-4-yl)-6-chloro-1H-indazole-3-carboxamide (32)

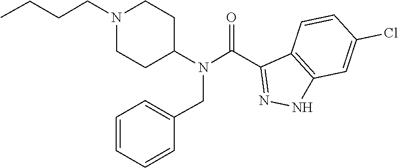 [SMILES: CCCCN1CCC(N(Cc2ccccc2)C(=O)c2n[nH]c3cc(Cl)ccc23)CC1]

Yield 14%, Yellow gel, ¹H NMR (500 MHz, Chloroform-d) δ 10.43 (s, 1H), 8.16-8.00 (m, 1H), 7.40-7.09 (m, 7H), 5.24 (s, 1H), 4.85 (s, 2H), 3.33-3.02 (m, 2H), 2.71-1.93 (m, 5H), 1.79 (s, 2H), 1.71-1.48 (m, 3H), 1.41-1.29 (m, 2H), 0.95-0.82 (m, 3H).

### Synthesis of N-benzyl-N-(1-butylpiperidin-4-yl)-6-(trifluoromethyl)-1H-indazole-3-carboxamide (33)

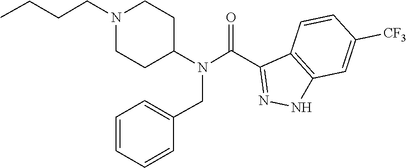 [SMILES: CCCCN1CCC(N(Cc2ccccc2)C(=O)c2n[nH]c3cc(C(F)(F)F)ccc23)CC1]

Yield 12%, Pale yellow sticky gel, ¹H NMR (500 MHz, Chloroform-d) δ 11.34 (s, 1H), 8.43-8.26 (m, 1H), 7.90-7.76 (m, 1H), 7.52-7.46 (m, 1H), 7.40-7.30 (m, 1H), 7.26 (s, 1H), 7.23-7.07 (m, 3H), 5.26 (s, 1H), 4.94-4.66 (m, 2H), 3.30-2.94 (m, 2H), 2.66-2.46 (m, 1H), 2.46-2.24 (m, 2H), 2.24-1.92 (m, 3H), 1.92-1.71 (m, 3H), 1.53-1.42 (m, 1H), 1.35-1.22 (m, 2H), 0.96-0.83 (m, 3H).

### Synthesis of N-benzyl-N-(1-butylpiperidin-4-yl)-5,6-dichloro-1H-indazole-3-carboxamide (34)

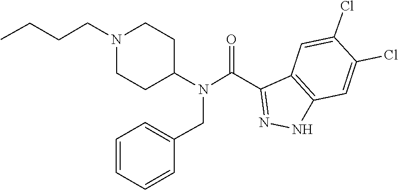 [SMILES: CCCCN1CCC(N(Cc2ccccc2)C(=O)c2n[nH]c3cc(Cl)c(Cl)cc23)CC1]

Yield 11%, Pale yellow sticky gel, ¹H NMR (500 MHz, Chloroform-d) δ 11.13 (s, 1H), 8.45-8.35 (m, 1H), 7.69-7.57 (m, 1H), 7.36-7.24 (m, 2H), 7.24-7.08 (m, 3H), 5.26 (s, 1H), 4.81 (s, 2H), 3.29-2.97 (m, 2H), 2.65-1.92 (m, 5H), 1.92-1.41 (m, 5H), 1.42-1.20 (m, 2H), 1.00-0.79 (m, 3H).

### Synthesis of N-benzyl-N-(1-butylpiperidin-4-yl)-7-chloroisoquinoline-3-carboxamide (35)

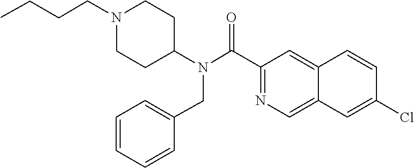 [SMILES: CCCCN1CCC(N(Cc2ccccc2)C(=O)c2cc3ccc(Cl)cc3cn2)CC1]

Yield 72%, Pale yellow sticky gel, H NMR (500 MHz, Chloroform-d) δ 9.18 (s, 0.5H), 9.05 (s, 0.5H), 8.08 (s, 1H), 8.02 (s, 0.5H), 7.93 (m, 1H), 7.87 (d, J=8.7 Hz, 0.5H), 7.77-7.68 (m, 1H), 7.61 (d, J=8.4 Hz, 0.5H), 7.40 (d, J=7.0 Hz, 1H), 7.32 (t, J=7.3 Hz, 1H), 7.25-7.20 (m, 0.5H), 7.16 (m, 1.5H), 7.11 (s, 0.5H), 4.83 (s, 2H), 4.71 (brs, 0.5H), 3.91 (brs, 0.5H), 3.16-3.03 (brs, 1H), 2.91 (brs, 1H), 2.42 (brs, 1H), 2.22 (brs, 2H), 1.96-1.77 (m, 3H), 1.73-1.48 (m, 2H), 1.44-1.36 (m, 1H), 1.36-1.20 (m, 3H), 0.96-0.82 (m, 3H).

### Synthesis of N-(1-butylpiperidin-4-yl)-N-(4-fluorobenzyl)-1H-indazole-3-carboxamide (36)

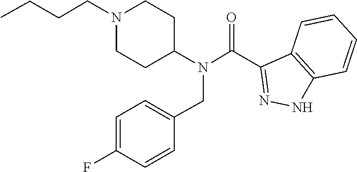 [SMILES: CCCCN1CCC(N(Cc2ccc(F)cc2)C(=O)c2n[nH]c3ccccc23)CC1]

Yield 9%, Yellow gel, H NMR (500 MHz, Chloroform-d) δ 10.54 (s, 1H), 8.28-8.10 (m, 1H), 7.54-7.37 (m, 2H), 7.34-7.26 (m, 2H), 7.17 (s, 1H), 7.01-6.92 (m, 1H), 6.88 (s, 1H), 5.09 (s, 1H), 4.79 (s, 1H), 4.73 (s, 1H), 3.04-2.90 (m, 2H), 2.37-2.23 (m, 2H), 2.14-2.02 (m, 1H), 1.92-1.69 (m, 5H), 1.50-1.37 (m, 2H), 1.33-1.26 (m, 2H), 0.88 (s, 3H).

LRMS (ESI+) m/z calcd for C₂₅H₃₁N₄O₃S⁺ [M+H]⁺ 409.2. found 409.2

### Synthesis of N-(1-butylpiperidin-4-yl)-6-chloro-N-(4-fluorobenzyl)-1H-indazole-3-carboxamide (37)

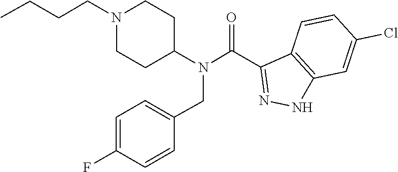 [SMILES: CCCCN1CCC(N(Cc2ccc(F)cc2)C(=O)c2n[nH]c3cc(Cl)ccc23)CC1]

Yield 27%, Yellow gel, ¹H NMR (500 MHz, Chloroform-d) δ 10.48 (s, 1H), 10.28 (s, 1H), 8.22 (d, J=7.4 Hz, 1H), 7.54-7.38 (m, 2H), 7.38-7.17 (m, 4H), 5.16 (s, 1H), 4.96-4.65 (m, 2H), 3.89-3.73 (m, 2H), 2.94-2.77 (m, 3H), 2.77-2.58 (m, 1H), 1.96-1.80 (m, 4H), 1.76 (s, 2H), 1.48-1.36 (m, 2H), 0.94 (t, J=7.1 Hz, 3H).

### Synthesis of N-(1-butylpiperidin-4-yl)-N-(4-fluorobenzyl)-5-methyl-1H-indazole-3-carboxamide (38)

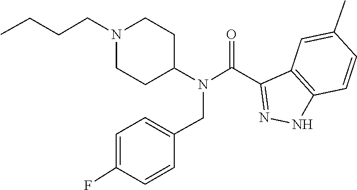 [SMILES: CCCCN1CCC(N(Cc2ccc(F)cc2)C(=O)c2n[nH]c3ccc(C)cc23)CC1]

Yield 22%, Yellow gel, ¹H NMR (500 MHz, Chloroform-d) δ 10.45 (s, 1H), 8.06-7.92 (m, 1H), 7.44-7.30 (m, 2H), 7.27-7.18 (m, 2H), 7.01-6.82 (m, 2H), 5.17 (s, 1H), 4.80 (s, 2H), 3.27-2.98 (m, 2H), 2.59-2.23 (m, 6H), 2.15-1.92 (m, 2H), 1.86-1.43 (m, 5H), 1.39-1.26 (m, 2H), 0.98-0.82 (m, 3H).

### Synthesis of N-(1-butylpiperidin-4-yl)-5-chloro-N-(4-fluorobenzyl)-1H-indazole-3-carboxamide (39)

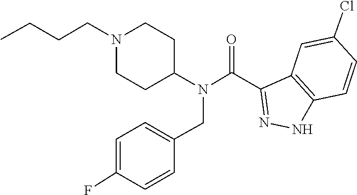 [SMILES: CCCCN1CCC(N(Cc2ccc(F)cc2)C(=O)c2n[nH]c3ccc(Cl)cc23)CC1]

Yield 24%, Yellow gel, ¹H NMR (500 MHz, Chloroform-d) δ 10.63 (s, 1H), 8.34-8.15 (m, 1H), 7.45-7.34 (m, 2H), 7.30 (s, 1H), 7.16 (s, 1H), 7.02-6.85 (m, 2H), 5.10 (s, 1H), 4.84-4.65 (m, 2H), 3.06-2.90 (m, 2H), 2.37-2.21 (m, 2H), 2.12-2.01 (m, 1H), 1.95-1.52 (m, 5H), 1.48-1.38 (m, 2H), 1.33-1.26 (m, 2H), 0.89 (t, J=7.2 Hz, 3H).

### Synthesis of N-(1-butylpiperidin-4-yl)-5-fluoro-N-(4-fluorobenzyl)-1H-indazole-3-carboxamide (40)

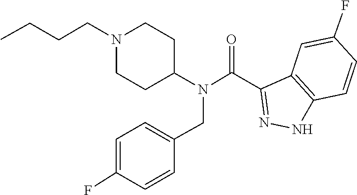 [SMILES: CCCCN1CCC(N(Cc2ccc(F)cc2)C(=O)c2n[nH]c3ccc(F)cc23)CC1]

Yield 31%, Yellow gel, ¹H NMR (500 MHz, Chloroform-d) δ 10.61 (s, 1H), 7.95-7.80 (m, 1H), 7.51-7.35 (m, 1H), 7.33-7.27 (m, 1H), 7.24-7.11 (m, 2H), 7.00-6.84 (m, 2H), 5.12 (s, 1H), 4.86-4.65 (m, 2H), 2.95 (s, 2H), 2.38-2.24 (m, 2H), 2.14-2.03 (m, 1H), 1.94-1.59 (m, 5H), 1.44 (s, 2H), 1.34-1.26 (m, 2H), 0.89 (t, J=7.3 Hz, 3H).

### Synthesis of N-(1-butylpiperidin-4-yl)-N-(4-fluorobenzyl)isoquinoline-3-carboxamide (41)

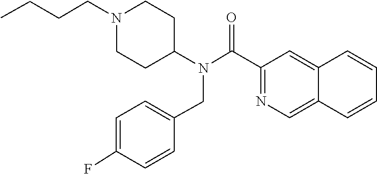 [SMILES: CCCCN1CCC(N(Cc2ccc(F)cc2)C(=O)c2cc3ccccc3cn2)CC1]

Yield 33%, Yellow gel, ¹H NMR (500 MHz, Chloroform-d) δ 9.24 (s, 1H), 9.12 (s, 1H), 8.10-7.89 (m, 3H), 7.84-7.61 (m, 2H), 7.43-7.32 (m, 1H), 7.23-7.14 (m, 1H), 7.05-6.97 (m, 1H), 6.87 (t, J=7.5 Hz, 1H), 4.81 (s, 2H), 4.73-4.42 (m, 0.5H), 3.93 (s, 0.5H), 3.08 (s, 1H), 2.91 (s, 1H), 2.50-2.14 (m, 3H), 2.02-1.48 (m, 5H), 1.45-1.21 (m, 4H), 0.93-0.82 (m, 3H).

### Synthesis of N-(1-butylpiperidin-4-yl)-N-(pyridin-3-ylmethyl)-1H-indazole-3-carboxamide (42)

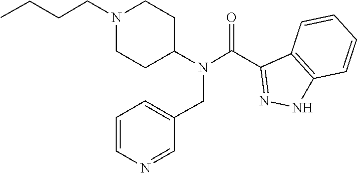 [SMILES: CCCCN1CCC(N(Cc2cccnc2)C(=O)c2n[nH]c3ccccc23)CC1]

Yield 9%, Yellow gel, H NMR (500 MHz, Chloroform-d) δ 11.07 (s, 1H), 8.68-8.35 (m, 2H), 8.27-8.08 (m, 1H), 7.80-7.35 (m, 3H), 7.29-7.09 (m, 2H), 5.21 (s, 1H), 4.84 (s, 2H), 3.26-2.97 (m, 2H), 2.58-2.23 (m, 3H), 2.19-1.95 (m, 3H), 1.89-1.74 (m, 2H), 1.63-1.45 (m, 2H), 1.38-1.26 (m, 2H), 0.94-0.78 (m, 3H).

### Synthesis of N-(1-butylpiperidin-4-yl)-6-chloro-N-(pyridin-3-ylmethyl)-1H-indazole-3-carboxamide (43)

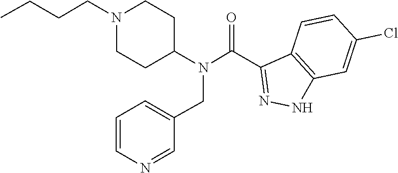 [SMILES: CCCCN1CCC(N(Cc2cccnc2)C(=O)c2n[nH]c3cc(Cl)ccc23)CC1]

Yield 19%, Yellow gel, ¹H NMR (500 MHz, Chloroform-d) δ 10.92 (m, 1H), 8.67-8.38 (m, 2H), 8.23-8.05 (m, 1H), 7.64 (m, 1H), 7.54-7.41 (m, 1H), 7.23 (dd, J=8.7, 1.6 Hz, 1H), 7.15 (s, 1H), 5.18 (s, 1H), 4.82 (s, 2H), 3.17-2.87 (m, 2H), 2.50-2.11 (m, 3H), 2.11-1.38 (m, 7H), 1.38-1.26 (m, 2H), 0.97-0.78 (m, 3H).

### Synthesis of N-(1-butylpiperidin-4-yl)-5-methyl-N-(pyridin-3-ylmethyl)-1H-indazole-3-carboxamide (44)

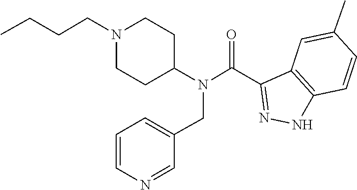 [SMILES: CCCCN1CCC(N(Cc2cccnc2)C(=O)c2n[nH]c3ccc(C)cc23)CC1]

Yield 8%, Yellow gel, ¹H NMR (500 MHz, Chloroform-d) δ 10.42 (s, 1H), 8.67-8.38 (m, 2H), 8.09-7.91 (m, 1H), 7.77-7.59 (m, 1H), 7.46-7.12 (m, 3H), 5.19 (s, 1H), 4.82 (s, 2H), 2.97 (s, 2H), 2.47 (s, 2H), 2.39-2.26 (m, 2H), 2.13 (s, 1H), 1.95-1.61 (m, 6H), 1.51-1.40 (m, 2H), 1.33-1.26 (m, 2H), 0.93-0.85 (m, 3H).

### Synthesis of N-(1-butylpiperidin-4-yl)-5-chloro-N-(pyridin-3-ylmethyl)-1H-indazole-3-carboxamide (45)

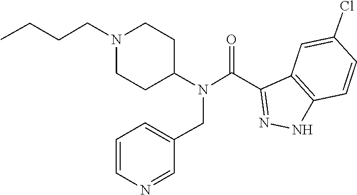 [SMILES: CCCCN1CCC(N(Cc2cccnc2)C(=O)c2n[nH]c3ccc(Cl)cc23)CC1]

Yield 26%, Yellow gel, ¹H NMR (500 MHz, Chloroform-a) δ 11.39 (m, 1H), 8.66-8.37 (m, 4H), 8.29 (s, 0.5H), 8.17 (s, 0.5H), 7.71 (s, 0.5H), 7.56 (s, 0.5H), 7.46-7.31 (m, 2H), 7.25-7.10 (m, 1H), 5.18 (s, 1H), 4.92-4.69 (m, 2H), 3.15-2.93 (m, 2H), 2.49-2.28 (m, 2H), 2.21-2.09 (m, 1H), 1.95 (s, 3H), 1.84-1.70 (m, 2H), 1.54-1.41 (m, 2H), 1.33-1.21 (m, 2H), 0.98-0.81 (m, 3H).

### Synthesis of N-(1-butylpiperidin-4-yl)-5-fluoro-N-(pyridin-3-ylmethyl)-1H-indazole-3-carboxamide (46)

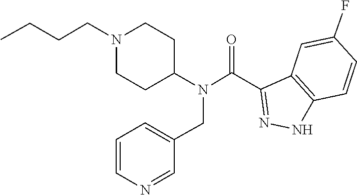 [SMILES: CCCCN1CCC(N(Cc2cccnc2)C(=O)c2n[nH]c3ccc(F)cc23)CC1]

Yield 22%, Yellow gel, ¹H NMR (500 MHz, Chloroform-d) 11.59 (s, 1H), 8.67-8.49 (m, 1H), 8.49-8.35 (m, 1H), 7.97-7.54 (m, 2H), 7.45 (m, Hz, 1H), 7.24-7.08 (m, 2H), 5.24 (s, 1H), 5.00-4.74 (m, 2H), 3.28-2.99 (m, 2H), 2.62-2.23 (m, 4H), 2.20-1.99 (m, 3H), 1.86-1.73 (m, 2H), 1.64-1.46 (m, 2H), 1.35-1.25 (m, 2H), 0.98-0.81 (m, 3H).

LRMS (ESI+) m/z calcd for C₂₅H₃₁N₄O₃S⁺ [M+H]⁺ 409.2. found 409.2.2

### Synthesis of N-(1-butylpiperidin-4-yl)-N-(pyridin-3-ylmethyl)isoquinoline-3-carboxamide (47)

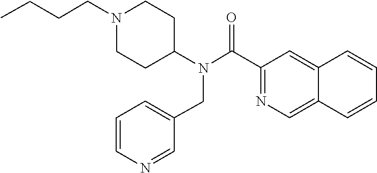 [SMILES: CCCCN1CCC(N(Cc2cccnc2)C(=O)c2cc3ccccc3cn2)CC1]

Yield 79%, Yellow gel, ¹H NMR (500 MHz, Chloroform-d) δ 9.23 (s, 0.6H), 9.08 (s, 0.4H), 8.67 (s, 0.6H), 8.49 (s, 1H), 8.38 (s, 0.4H), 8.09 (s, 1H), 8.04 (d, J=7.6 Hz, 1H), 7.93 (d, J=7.6 Hz, 1H), 7.87-7.74 (m, 2H), 7.74-7.68 (m, 1H), 7.65 (s, 0.6H), 7.14 (s, 0.4H), 4.90 (s, 0.7H), 4.83 (s, 1.3H), 4.02 (brs, 1H), 3.15 (brs, 1H), 2.98 (brs, 1H), 2.50 (brs, 1H), 2.36-2.22 (m, 2H), 2.09-1.94 (m, 2H), 1.92-1.78 (m, 3H), 1.67 (brs, 2H), 1.49-1.39 (m, 1H), 1.36-1.26 (m, 2H), 0.97-0.83 (m, 3H).

### Synthesis of 1-benzyl-3-(3,4-dichlorophenyl)-1-(1-(2-hydroxypropyl)piperidin-4-yl)urea (51)

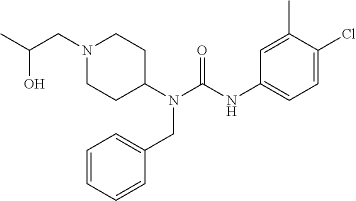 [SMILES: Cc1cc(NC(=O)N(Cc2ccccc2)C2CCN(CC(C)O)CC2)ccc1Cl]

Yield 12%, Yellow gel, ¹H NMR (500 MHz, Chloroform-d) δ 7.45-7.40 (m, 2H), 7.40 (d, J=2.5 Hz, 1H), 7.36 (d, J=7.3 Hz, 1H), 7.33 (d, J=7.2 Hz, 2H), 7.22 (d, J=8.7 Hz, 1H), 6.88 (dd, J=8.8, 2.6 Hz, 1H), 6.20 (s, 1H), 4.50 (s, 2H), 4.49-4.42 (m, 1H), 3.82 (s, 1H), 3.11 (d, J=10.6 Hz, 1H), 2.96-2.88 (m, 1H), 2.48 (t, J=10.9 Hz, 1H), 2.34 (d, J=10.7 Hz, 1H), 2.31-2.23 (m, 1H), 2.13 (t, J=11.0 Hz, 1H), 1.88-1.81 (m, 2H), 1.80-1.66 (m, 2H), 1.12 (d, J=6.1 Hz, 3H).

LRMS (ESI+) m/z calcd for C₂₅H₃₁N₄O₃S⁺ [M+H]⁺ 436.1. found 438.0.2

### Synthesis of 1-benzyl-3-(3,4-dichlorophenyl)-1-(1-(oxetan-3-yl)piperidin-4-yl)urea (52)

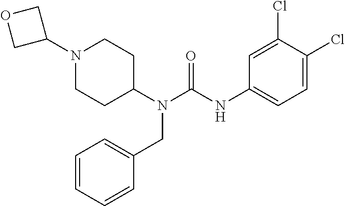 [SMILES: O=C(Nc1ccc(Cl)c(Cl)c1)N(Cc1ccccc1)C1CCN(C2COC2)CC1]

Yield 18%, Yellow gel, ¹H NMR (400 MHz, Chloroform-d) δ 7.44-7.37 (m, 3H), 7.37-7.28 (m, 3H), 7.20 (d, J=8.7 Hz, 1H), 6.88 (dd, J=8.7, 2.6 Hz, 1H), 6.21 (s, 1H), 4.62 (t, J=6.6 Hz, 2H), 4.53 (t, J=6.2 Hz, 2H), 4.49 (s, 2H), 4.47-4.39 (m, 1H), 3.44 (p, J=6.4 Hz, 1H), 2.78 (d, J=11.5 Hz, 2H), 1.95 (td, J=11.7, 2.1 Hz, 2H), 1.82 (dd, J=11.8, 2.4 Hz, 2H), 1.78-1.65 (m, 2H).

LRMS (ESI+) m/z calcd for C₂₅H₃₁N₄O₃S⁺ [M+H]⁺ 434.1. found 434.4.2

### Synthesis of 1-benzyl-3-(3,4-dichlorophenyl)-1-(1-(2-fluoroethyl)piperidin-4-yl)urea (53)

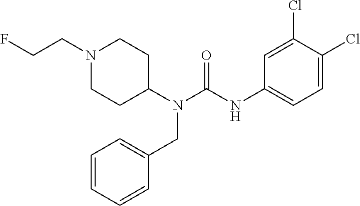 [SMILES: O=C(Nc1ccc(Cl)c(Cl)c1)N(Cc1ccccc1)C1CCN(CCF)CC1]

Yield 24%, Yellow gel, ¹H NMR (500 MHz, Chloroform-) 6 7.44-7.39 (m, 3H), 7.37-7.34 (m, 1H), 7.34-7.30 (m, 2H), 7.22 (d, J=8.7 Hz, 1H), 6.90 (dd, J=8.8, 2.6 Hz, 1H), 6.20 (s, 1H), 4.59 (t, J=4.9 Hz, 1H), 4.50 (s, 2H), 4.50-4.40 (m, 2H), 3.04 (d, J=11.5 Hz, 2H), 2.70 (dt, J=28.1, 4.8 Hz, 2H), 2.24 (t, J=10.8 Hz, 2H), 1.87-1.70 (m, 4H).

### Synthesis of 1-benzyl-3-(3,4-dichlorophenyl)-1-(1-(2,2-difluoroethyl)piperidin-4-yl)urea (54)

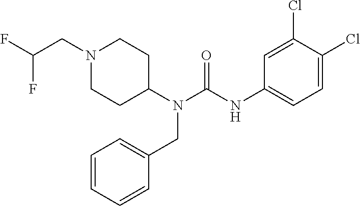 [SMILES: O=C(Nc1ccc(Cl)c(Cl)c1)N(Cc1ccccc1)C1CCN(CC(F)F)CC1]

Yield 49%, Yellow gel, ¹H NMR (500 MHz, Chloroform-d) δ 7.44-7.40 (m, 2H), 7.39 (d, J=2.6 Hz, 1H), 7.37-7.33 (m, 1H), 7.33-7.30 (m, 2H), 6.89 (dd, J=8.8, 2.6 Hz, 1H), 6.20 (s, 1H), 5.83 (tt, J=56.0, 4.3 Hz, 1H), 4.49 (s, 2H), 4.48-4.40 (m, 1H), 2.99 (dd, J=9.5, 2.1 Hz, 2H), 2.73 (td, J=15.0, 4.3 Hz, 2H), 2.37 (td, J=11.7, 2.6 Hz, 2H), 1.83-1.77 (m, 2H), 1.76-1.69 (m, 2H).

### Synthesis of 1-benzyl-3-(3,4-dichlorophenyl)-1-(1-(2,2-difluoropropyl)piperidin-4-yl)urea (55)

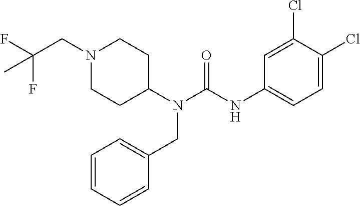 [SMILES: CC(F)(F)CN1CCC(N(Cc2ccccc2)C(=O)Nc2ccc(Cl)c(Cl)c2)CC1]

Yield 38%, Yellow gel, ¹H NMR (500 MHz, Chloroform-d) δ 7.45-7.40 (m, 2H), 7.39 (d, J=2.5 Hz, 1H), 7.37-7.31 (m, 3H), 7.22 (d, J=8.7 Hz, 1H), 6.89 (dd, J=8.8, 2.6 Hz, 1H), 6.19 (s, 1H), 4.49 (s, 2H), 4.45-4.36 (m, 1H), 3.01 (d, J=11.7 Hz, 2H), 2.66 (t, J=13.7 Hz, 2H), 2.39 (td, J=11.6, 2.6 Hz, 2H), 1.82-1.69 (m, 4H), 1.60 (t, J=18.7 Hz, 3H).

### Synthesis of 1-benzyl-3-(3,4-dichlorophenyl)-1-(1-(2-fluoropropyl)piperidin-4-yl)urea (56)

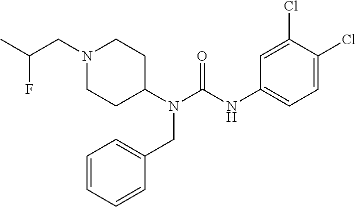 [SMILES: CC(F)CN1CCC(N(Cc2ccccc2)C(=O)Nc2ccc(Cl)c(Cl)c2)CC1]

Yield 9%, Yellow gel, ¹H NMR (500 MHz, Chloroform-d) δ 7.44-7.39 (m, 3H), 7.37-7.33 (m, 1H), 7.33-7.30 (m, 2H), 7.22 (d, J=8.8 Hz, 1H), 6.90 (dd, J=8.8, 2.5 Hz, 1H), 6.20 (s, 1H), 4.91-4.73 (m, 1H), 4.50 (s, 2H), 4.48-4.39 (m, 1H), 3.07-2.99 (m, 2H), 2.68-2.58 (m, 1H), 2.51-2.34 (m, 1H), 2.31-2.19 (m, 2H), 1.88-1.71 (m, 4H), 1.31 (dd, J=23.6, 6.3 Hz, 3H).

### Synthesis of 1-benzyl-1-(1-(butylsulfonyl)piperidin-4-yl)-3-(3,4-dichlorophenyl)urea ((57)

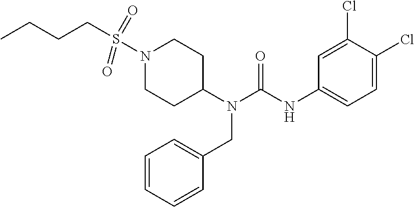 [SMILES: CCCCS(=O)(=O)N1CCC(N(Cc2ccccc2)C(=O)Nc2ccc(Cl)c(Cl)c2)CC1]

Yield 48%, Pale yellow solid, ¹H NMR (400 MHz, CDCl₃) δ 7.46-7.33 (m, 4H), 7.33-7.29 (m, 2H), 7.23 (d, J=8.8 Hz, 1H), 6.89 (dd, J=8.8, 2.6 Hz, 1H), 6.28 (s, 1H), 4.61-4.53 (m, 1H), 4.49 (s, 2H), 3.90 (dt, J=12.4, 2.4 Hz, 2H), 2.94-2.84 (m, 4H), 1.93-1.85 (m, 2H), 1.80-1.72 (m, 4H), 1.49-1.40 (m, 2H), 0.95 (t, J=7.4 Hz, 3H). ¹³C NMR (100 MHz, CDCl3) δ 155.3, 138.4, 136.7, 132.5, 130.2, 129.5, 128.4, 126.2, 126.0, 121.4, 119.0, 52.1, 49.7, 46.3, 45.7, 30.1, 25.1, 21.7, 13.6. LRMS (ESI+) m/z calcd for C₂₃H₃₀Cl₂N₃O₃S⁺ [M+H]⁺ 498.13. found 498.5.

### Synthesis of N-benzyl-N-(1-(butylsulfonyl)piperidin-4-yl)-5-chloro-1H-indazole-3-carboxamide (61)

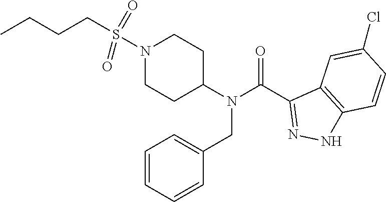 [SMILES: CCCCS(=O)(=O)N1CCC(N(Cc2ccccc2)C(=O)c2n[nH]c3ccc(Cl)cc23)CC1]

Yield 23%, Yellow gel, H NMR (500 MHz, Chloroform-d) δ 10.32 (s, 0.5H), 10.15 (s, 0.5H), 8.16 (d, J=8.3 Hz, 1H), 7.54-7.42 (m, 1H), 7.38-7.16 (m, 6H), 5.16 (s, 1H), 4.95-4.70 (m, 2H), 3.89-3.76 (m, 2H), 2.90-2.79 (m, 3H), 2.76-2.66 (m, 1H), 1.95-1.80 (m, 4H), 1.80-1.71 (m, 2H), 1.49-1.39 (m, 2H), 0.94 (t, J=7.3 Hz, 3H).

### Synthesis of N-benzyl-N-(1-(butylsulfonyl)piperidin-4-yl)-1H-indazole-3-carboxamide (62)

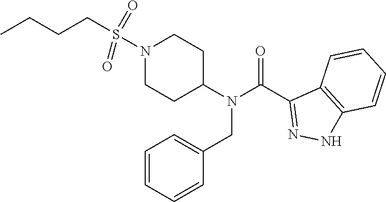 [SMILES: CCCCS(=O)(=O)N1CCC(N(Cc2ccccc2)C(=O)c2n[nH]c3ccccc23)CC1]

Yield 26%, Yellow gel, ¹H NMR (500 MHz, Chloroform-d) δ 10.48 (s, 1H), 10.28 (s, 1H), 8.27-8.16 (m, 1H), 7.54-7.38 (m, 2H), 7.38-7.18 (m, 6H), 5.16 (s, 1H), 4.97-4.62 (m, 2H), 3.91-3.70 (m, 2H), 2.91-2.75 (m, 3H), 2.72-2.59 (m, 1H), 1.96-1.79 (m, 4H), 1.76 (s, 2H), 1.51-1.35 (m, 2H), 0.94 (t, J=7.1 Hz, 3H).

### Synthesis of N-benzyl-N-(1-(butylsulfonyl)piperidin-4-yl)isoquinoline-3-carboxamide (63)

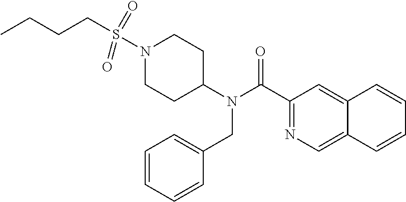 [SMILES: CCCCS(=O)(=O)N1CCC(N(Cc2ccccc2)C(=O)c2cc3ccccc3cn2)CC1]

Yield 82%, Yellow gel, ¹H NMR (500 MHz, Chloroform-d) δ 9.23-9.14 (m, 1H), 8.14-7.60 (m, 5H), 7.43-7.30 (m, 2H), 7.25-7.12 (m, 3H), 4.81 (s, 2H), 4.63 (s, 1H), 4.16-4.07 (m, 1H), 3.92-3.81 (m, 1H), 3.81-3.69 (m, 1H), 2.87 (d, J=6.4 Hz, 2H), 2.82-2.73 (m, 1H), 2.51 (t, J=11.5 Hz, 1H), 1.97-1.63 (m, 6H), 1.57 (s, 4H), 1.49-1.32 (m, 2H), 1.01-0.84 (m, 3H).

### Synthesis of N-(1-(butylsulfonyl)piperidin-4-yl)-N-(2,3-dihydro-1H-inden-1-yl)isoquinoline-3-carboxamide (64)

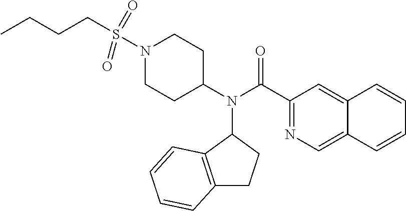 [SMILES: CCCCS(=O)(=O)N1CCC(N(C(=O)c2cc3ccccc3cn2)C2CCc3ccccc32)CC1]

Yield 39%, Yellow gel, ¹H NMR (500 MHz, Chloroform-d) δ 9.19 (brs, 1H), 8.01-7.93 (m, 1.5H), 7.85 (d, J=7.6 Hz, 1H), 7.72 (t, J=7.4 Hz, 1H), 7.68-7.62 (m, 1H), 7.51-7.44 (m, 0.5H), 7.25-7.12 (m, 3H), 5.46 (s, 1H), 5.02 (s, 0.5H), 3.90 (s, 0.5H), 3.37-2.87 (m, 5H), 2.70 (brs, 1.5H), 2.50-2.05 (m, 4.5H), 1.98-1.43 (m, 5H), 1.40-1.27 (m, 2H), 0.97-0.85 (m, 3H).

### Synthesis of (R)—N-(1-(butylsulfonyl)piperidin-4-yl)-N-(1-phenylethyl)isoquinoline-3-carboxamide (65)

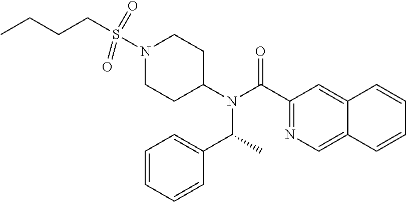 [SMILES: CCCCS(=O)(=O)N1CCC(N(C(=O)c2cc3ccccc3cn2)[C@H](C)c2ccccc2)CC1]

Yield 12%, Yellow gel, ¹H NMR (500 MHz, Chloroform-d) δ 9.25 (s, 1H), 8.03-7.98 (m, 2H), 7.91 (d, J=8.0 Hz, 1H), 7.76 (t, J=7.4 Hz, 1H), 7.68 (t, J=7.5 Hz, 1H), 7.56-7.40 (m, 2H), 7.33 (t, J=7.5 Hz, 2H), 7.30-7.26 (m, 1H), 5.29 (s, 1H), 4.83 (s, 0.5H), 3.84 (s, 0.5H), 3.32-2.71 (m, 5H), 2.43-1.55 (m, 9H), 1.45-1.28 (m, 2H), 1.04-0.85 (m, 3H).

### Synthesis of (S)—N-(1-(butylsulfonyl)piperidin-4-yl)-N-(1-phenylethyl)isoquinoline-3-carboxamide (66)

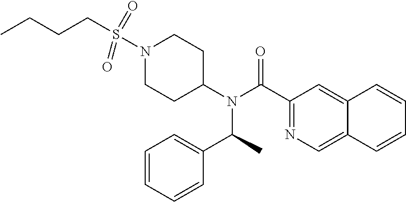 [SMILES: CCCCS(=O)(=O)N1CCC(N(C(=O)c2cc3ccccc3cn2)[C@@H](C)c2ccccc2)CC1]

Yield 13%, Yellow gel, ¹H NMR (500 MHz, Chloroform-d) δ 9.25 (s, 1H), 8.05-7.96 (m, 2H), 7.90 (d, J=8.0 Hz, 1H), 7.76 (t, J=7.4 Hz, 1H), 7.68 (t, J=7.4 Hz, 1H), 7.55-7.39 (m, 2H), 7.33 (t, J=7.5 Hz, 2H), 7.29-7.26 (m, 1H), 5.28 (s, 1H), 4.83 (s, 0.5H), 3.94-3.71 (m, 0.5H), 3.35-2.68 (m, 5H), 2.45-1.60 (m, 9H), 1.41-1.27 (m, 2H), 1.06-0.78 (m, 3H).

### Synthesis of N-(1-(butyl sulfonyl) piperidin-4-yl)-N-(pyridin-4-ylmethyl)isoquinoline-3-carboxamide (67)

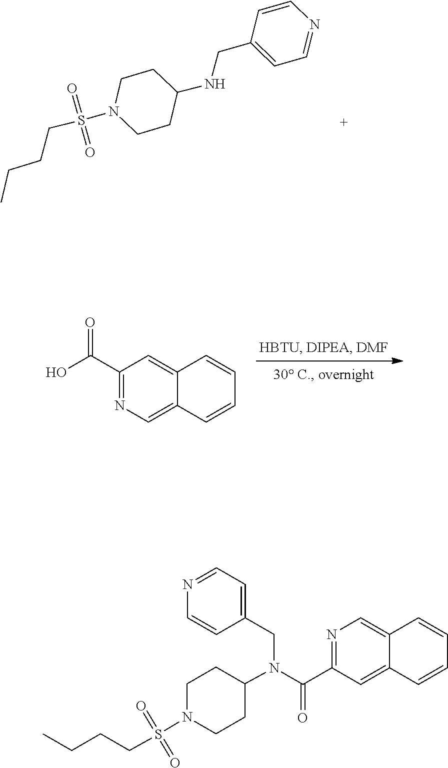 [SMILES: CCCCS(=O)(=O)N1CCC(NCc2ccncc2)CC1 ; O=C(O)c1cc2ccccc2cn1 ; CCCCS(=O)(=O)N1CCC(N(Cc2ccncc2)C(=O)c2cc3ccccc3cn2)CC1]

Yield 20%, Yellow oil, ¹H NMR (500 MHz, Chloroform-d) δ 9.25 (s, 0.6H), 9.06 (s, 0.4H), 8.65-8.41 (m, 2H), 8.20-7.63 (m, 5H), 7.32-7.20 (m, 2H), 4.88 (s, 1H), 4.77-4.75 (1.4H), 4.29-4.20 (m, 0.6H), 3.85 (dd, J=49.8, 12.3 Hz, 2H), 2.92-2.79 (m, 3H), 2.57 (t, J=12.3 Hz, 1H), 2.05-1.65 (m, 6H), 1.47-1.36 (m, 2H), 0.93 (dt, J=27.4, 7.4 Hz, 3H).

LRMS (ESI+) m/z calcd for C₂₅H₃₁N₄O₃S⁺ [M+H]⁺ 467.2. found 467.5.

### Synthesis of N-(1-(butylsulfonyl)piperidin-4-yl)-N-(pyridin-3-ylmethyl)isoquinoline-3-carboxamide (68)

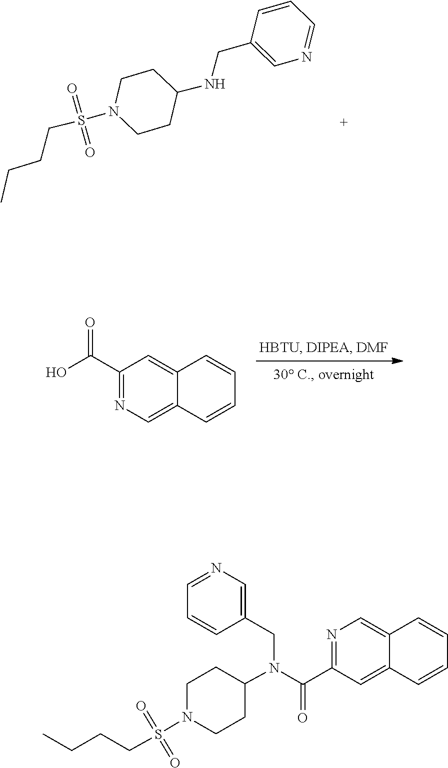 [SMILES: CCCCS(=O)(=O)N1CCC(NCc2cccnc2)CC1 ; O=C(O)c1cc2ccccc2cn1 ; CCCCS(=O)(=O)N1CCC(N(Cc2cccnc2)C(=O)c2cc3ccccc3cn2)CC1]

Yield 26%, Yellow oily solid, ¹H NMR (600 MHz, Chloroform-d) δ 9.23 (s, 0.55H), 9.10 (s, 0.45H), 8.73-8.37 (m, 2H), 8.20-7.61 (m, 6H), 7.26 (m, 1H), 4.83 (d, J=35.0 Hz, 2H), 4.66 (m, 0.45H), 4.19 (m, 0.55H), 3.84 (dd, J=56.9, 12.3 Hz, 2H), 2.94-2.77 (m, 3H), 2.55 (t, J=12.2 Hz, 1H), 2.04-1.64 (m, 6H), 1.48-1.33 (m, 2H), 1.01-0.82 (m, 3H). ¹³C NMR (151 MHz, Chloroform-d) δ 170.19, 169.95, 151.37, 151.16, 148.84, 148.72, 148.61, 148.10, 148.04, 136.14, 135.99, 135.10, 134.96, 134.41, 131.30, 131.18, 128.98, 128.84, 127.69, 127.58, 123.59, 123.30, 121.96, 121.83, 56.87, 53.53, 49.65, 47.05, 45.88, 45.61, 43.32, 31.27, 29.88, 25.20, 25.14, 21.84, 21.80, 13.69. LRMS (ESI+) m/z calcd for C₂₅H₃₁N₄O₃S⁺ [M+H]⁺ 467.2. found 467.5.

### Synthesis of N-(1-(butylsulfonyl)piperidin-4-yl)-N-(pyridin-2-ylmethyl)isoquinoline-3-carboxamide (69)

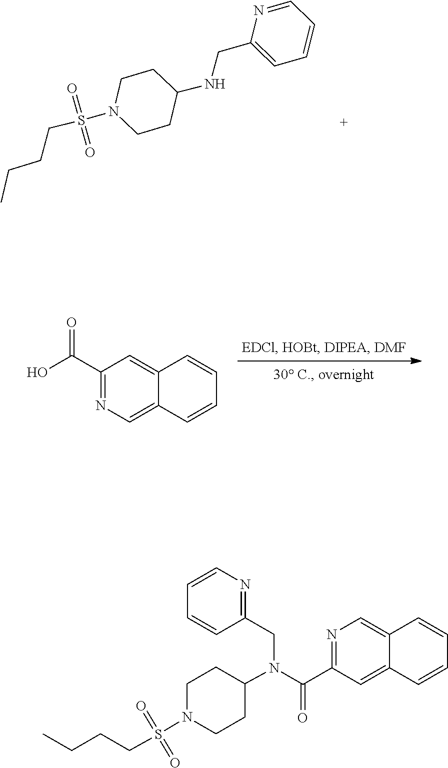 [SMILES: CCCCS(=O)(=O)N1CCC(NCc2ccccn2)CC1 ; O=C(O)c1cc2ccccc2cn1 ; CCCCS(=O)(=O)N1CCC(N(Cc2ccccn2)C(=O)c2cc3ccccc3cn2)CC1]

Yield 51%, Yellow oily solid, ¹H NMR (500 MHz, Chloroform-d) δ 9.17 (d, J=68.6 Hz, 1H), 8.61-8.42 (m, 1H), 8.12 (d, J=26.6 Hz, 1H), 7.94 (ddd, J=56.5, 53.1, 8.2 Hz, 2H), 7.80-7.52 (m, 3H), 7.43 (dd, J=69.4, 7.9 Hz, 1H), 7.14 (dt, J=43.5, 6.2 Hz, 1H), 4.91 (d, J=5.6 Hz, 2H), 4.71 (t, J−12.4 Hz, 0.5H), 4.15 (t, J=11.5 Hz, 0.5H), 3.81 (dd, J=51.6, 12.3 Hz, 2H), 2.96-2.84 (m, 2H), 2.84-2.50 (m, 2H), 1.99-1.68 (m, 6H), 1.42 (dq, J=29.1, 7.4 Hz, 2H), 0.93 (dt, J=23.3, 7.4 Hz, 3H).

LRMS (ESI+) m/z calcd for C₂₅H₃₁N₄O₃S⁺ [M+H]f 467.2. found 467.5.

### Synthesis of N-(1-(butylsulfonyl)piperidin-4-yl)-N-(3-fluorobenzyl)isoquinoline-3-carboxamide (70)

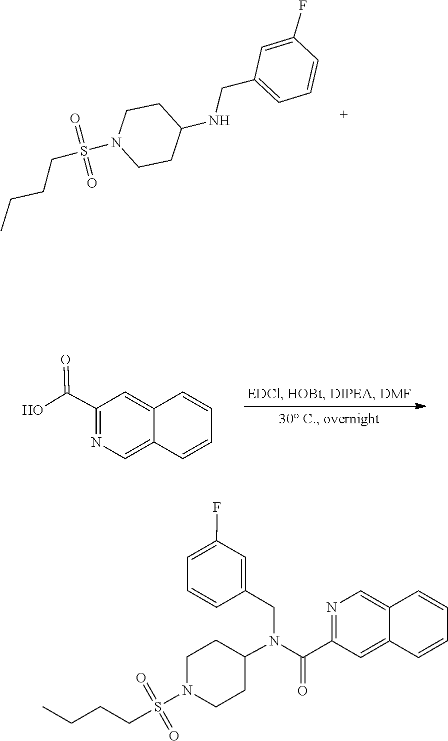 [SMILES: CCCCS(=O)(=O)N1CCC(NCc2cccc(F)c2)CC1 ; O=C(O)c1cc2ccccc2cn1 ; CCCCS(=O)(=O)N1CCC(N(Cc2cccc(F)c2)C(=O)c2cc3ccccc3cn2)CC1]

Yield 38%, White solid, ¹H NMR (400 MHz, Chloroform-d) δ 9.21 (m, 1H), 8.22-7.61 (m, 5H), 7.37-6.83 (m, 4H), 4.81 (m, 2H), 4.66 (s, 0.5H), 4.22-4.09 (m, 0.5H), 3.83 (dd, J=41.3, 13.1 Hz, 2H), 2.98-2.74 (m, 3H), 2.54 (t, J=12.1 Hz, 1H), 1.84 (m, 6H), 1.46-1.32 (m, 2H), 0.93 (m, 3H).

LRMS (ESI+) m/z calcd for C₂₆H₃₁FN₃O₃S⁺ [M+H]⁺ 484.2. found 484.7.

### Synthesis of N-(1-(butylsulfonyl)piperidin-4-yl)-N-(3-chlorobenzyl)isoquinoline-3-carboxamide (71)

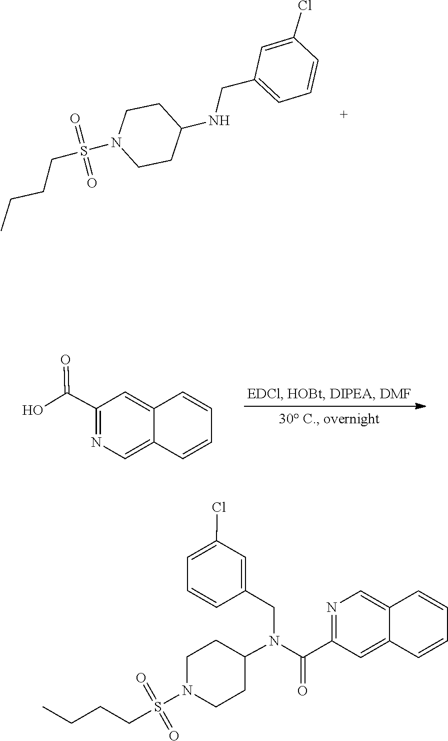 [SMILES: CCCCS(=O)(=O)N1CCC(NCc2cccc(Cl)c2)CC1 ; O=C(O)c1cc2ccccc2cn1 ; CCCCS(=O)(=O)N1CCC(N(Cc2cccc(Cl)c2)C(=O)c2cc3ccccc3cn2)CC1]

Yield 50%, White solid, ¹H NMR (400 MHz, Chloroform-d) δ 9.18 (m, 1H), 8.18-7.63 (m, 5H), 7.40-7.07 (m, 4H), 4.83-4.73 (m, 2H), 4.67 (s, 0.5H), 4.17 (s, 0.5H) 3.95-3.74 (m, 2H), 2.95 2.75 (m, 3H), 2.54 (t, J=12.2 Hz, 1H), 2.00-1.68 (m, 6H), 1.42-1.20 (m, 2H), 0.99-0.84 (m, 3H).

LRMS (ESI+) m/z calcd for C₂₆H₃₁ClN₃O₃S⁺ [M+H]⁺ 500.2. found 500.6.

### Synthesis of N-(3-bromobenzyl)-N-(1-(butylsulfonyl)piperidin-4-yl)isoquinoline-3-carboxamide (72)

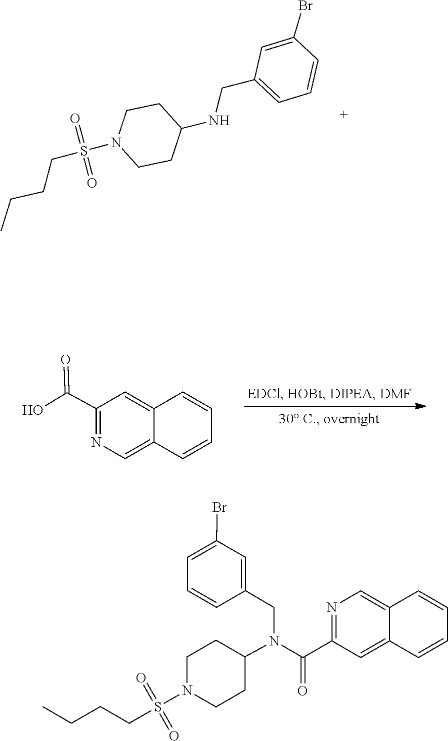 [SMILES: CCCCS(=O)(=O)N1CCC(NCc2cccc(Br)c2)CC1 ; O=C(O)c1cc2ccccc2cn1 ; CCCCS(=O)(=O)N1CCC(N(Cc2cccc(Br)c2)C(=O)c2cc3ccccc3cn2)CC1]

Yield 28%, White solid, ¹H NMR (500 MHz, Chloroform-d) δ 9.18 (d, J=56.2 Hz, 1H), 8.20-7.63 (m, 5H), 7.59-7.31 (m, 2H), 7.23-7.04 (m, 2H), 4.78 (d, J=6.2 Hz, 2H), 4.72-4.60 (m, 0.5H), 4.23-4.11 (m, 0.5H), 3.84 (dd, J=54.5, 11.0 Hz, 2H), 2.96-2.76 (m, 3H), 2.54 (t, J=12.0 Hz, 1H), 2.02-1.66 (m, 7H), 1.49-1.36 (m, 2H), 1.00-0.87 (m, 3H).

LRMS (ESI+) m/z calcd for C₂₆H₃₁BrN₃O₃S⁺ [M+H]⁺ 544.1. found 544.6.

### Synthesis of N-(1-(butylsulfonyl)piperidin-4-yl)-N-(thiophen-2-ylmethyl)isoquinoline-3-carboxamide (73)

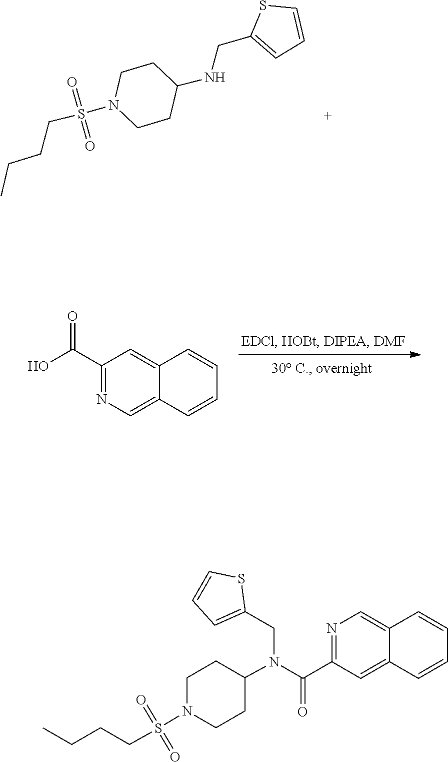 [SMILES: CCCCS(=O)(=O)N1CCC(NCc2cccs2)CC1 ; O=C(O)c1cc2ccccc2cn1 ; CCCCS(=O)(=O)N1CCC(N(Cc2cccs2)C(=O)c2cc3ccccc3cn2)CC1]

Yield 56%, White solid, H NMR (500 MHz, Chloroform-d) δ 9.21 (s, 1H), 8.20-7.96 (m, 2H), 7.90 (dd, J=23.9, 8.2 Hz, 1H), 7.84-7.64 (m, 2H), 7.23-7.07 (m, 1H), 6.99-6.85 (m, 1H), 6.79-6.77 (m, 1H), 4.94 (d, J=34.4 Hz, 2H), 4.91-4.89 (m, 0.5H), 4.04-4.03 (m, 0.5H), 3.93-3.68 (m, 2H), 2.93-2.68 (m, 3H), 2.54 (t, J=11.5 Hz, 1H), 2.02-1.89 (m, 4H), 1.85-1.68 (m, 2H), 1.51-1.37 (m, 2H), 1.01-0.83 (m, 3H).

LRMS (ESI+) m/z calcd for C₂₄H₃₀N₃O₃S₂⁺ [M+H]⁺ 472.2. found 472.5.

### Synthesis of N-(1-(butylsulfonyl)piperidin-4-yl)-N-(furan-2-ylmethyl)isoquinoline-3-carboxamide (74)

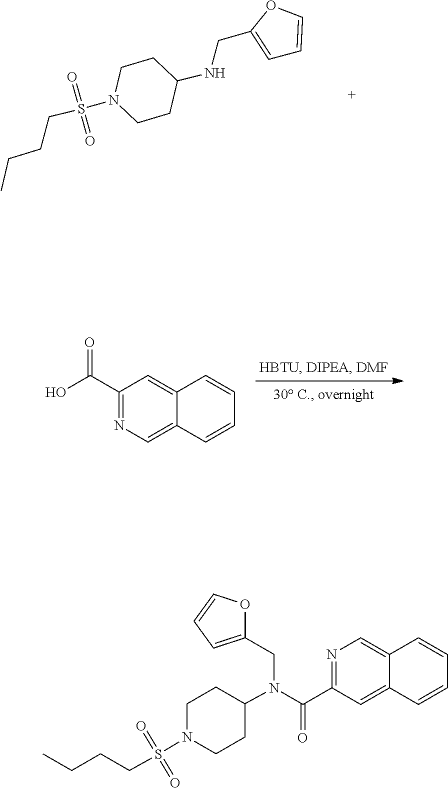 [SMILES: CCCCS(=O)(=O)N1CCC(NCc2ccco2)CC1 ; O=C(O)c1cc2ccccc2cn1 ; CCCCS(=O)(=O)N1CCC(N(Cc2ccco2)C(=O)c2cc3ccccc3cn2)CC1]

Yield 53%, Yellow oily solid, H NMR (500 MHz, Chloroform-d) δ 9.21 (s, 1H), 8.19-7.59 (m, 5H), 7.36-7.23 (m, 1H), 6.38 (d, J=35.6 Hz, 1H), 6.24-5.84 (m, 1H), 4.77-4.74 (m, 2H), 4.65-4.60 (m, 0.6H), 4.10-4.04 (m, 0.4H), 3.99-3.74 (m, 2H), 2.92-2.80 (m, 3H), 2.55 (s, 1H), 2.03-1.73 (m, 6H), 1.51-1.31 (m, 2H), 1.06-0.81 (m, 3H).

LRMS (ESI+) m/z calcd for C₂₄H₃₀N₃O₄S⁺ [M+H]⁺ 456.2. found 456.6.

### Synthesis of N-((1H-pyrrol-2-yl)methyl)-N-(1-(butyl sulfonyl) piperidin-4-yl) isoquinoline-3-carboxamide (75)

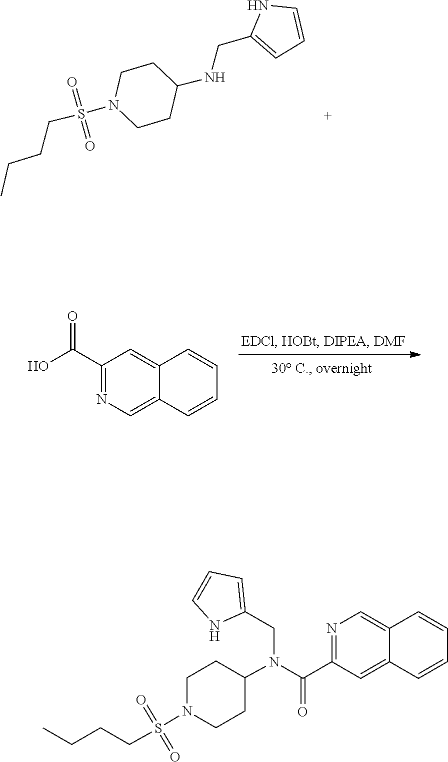 [SMILES: CCCCS(=O)(=O)N1CCC(NCc2ccc[nH]2)CC1 ; O=C(O)c1cc2ccccc2cn1 ; CCCCS(=O)(=O)N1CCC(N(Cc2ccc[nH]2)C(=O)c2cc3ccccc3cn2)CC1]

Yield 20%, Brown viscous liquid, NMR (600 MMz, Chloroform-d) 6 9.25 (s, 1H), 8.13 (s, 1H), 8.03 (d, J=8.0 Hz, 1H), 7.93 (d, J=8.4 Hz, 1H), 7.75 (dt, J=49.1, 7.8 Hz, 3H), 6.90 (q, J=2.1 Hz, 1H), 4.36 (s, 2H), 3.89-3.80 (m, 3H), 2.92-2.79 (m, 5H), 1.96-1.70 (m, 7H), 1.50-1.43 (m, 2H), 0.96 (t, J=7.4 Hz, 3H).

LRMS (ESI+) m/z calcd for C₂₄H₃₁N₄O₃S⁺ [M+H]⁺ 455.2. found 545.5.

### Synthesis of N-((1H-pyrazol-4-yl)methyl)-N-(1-(butyl sulfonyl) piperidin-4-yl) isoquinoline-3-carboxamide (76)

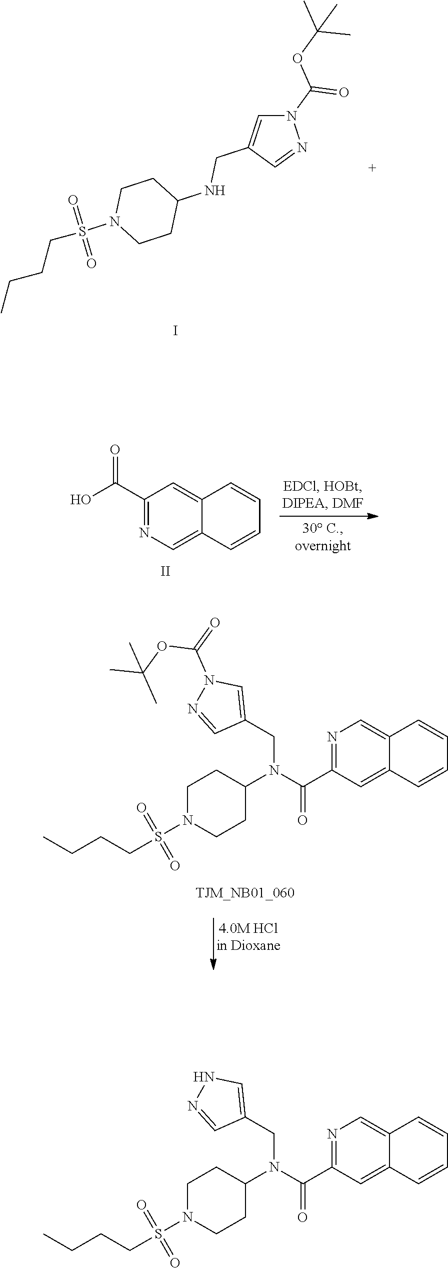

Yield 95%, Off-white flaky solid, ¹H NMR (600 MHz, DMSO-d₆) δ 9.33 (s, 1H), 8.20 (m, 1H), 8.12-8.02 (m, 2H), 7.85 (m, 1H), 7.77 (m, 1H), 7.64 (s, 1H), 7.42 (s, 1H), 4.49 (d, J=24.9 Hz, 2H), 2.86 (t, J=8.1 Hz, 2H), 1.96-1.81 (m, 2H), 1.80-1.71 (m, 2H), 1.63 (d, J=7.5 Hz, 1H), 1.54 (p, J=8.6, 8.2 Hz, 2H), 1.40 (q, J=7.3 Hz, 2H), 1.30 (q, J=7.5 Hz, 2H), 0.97-0.85 (m, 2H), 0.82 (t, J=7.4 Hz, 3H).

LRMS (ESI+) m/z calcd for C₂₃H₃₀N₅O₃S⁺ [M+H]⁺ 456.2. found 456.2.

### Synthesis of N-((1H-imidazol-5-yl)methyl)-N-(1-(butyl sulfonyl) piperidin-4-yl) isoquinoline-3-carboxamide (77)

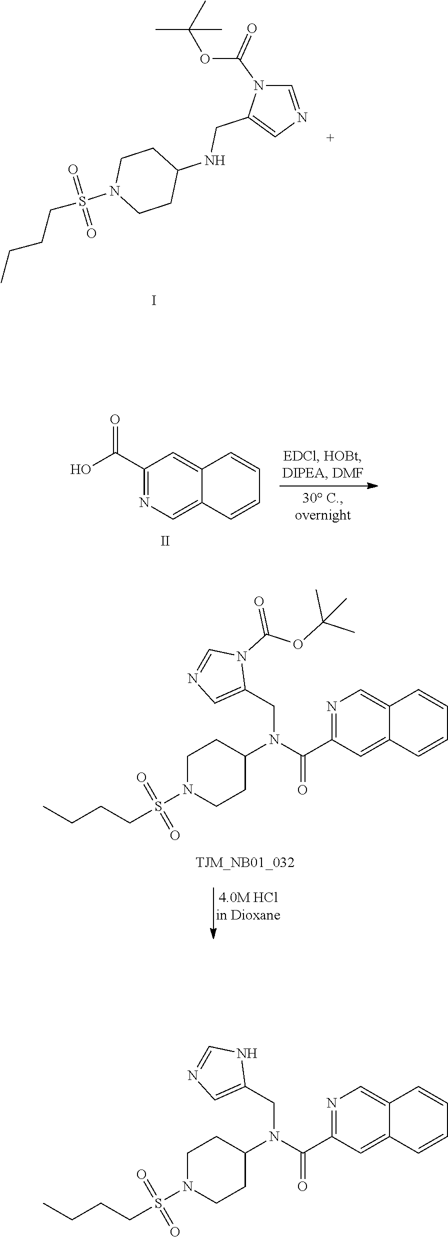

Yield 83%, Off-white flaky solid, ¹H NMR (600 MHz, DMSO-d6) δ 14.21 (d, J=55.4 Hz, 1H), 9.33 (d, J=28.8 Hz, 1H), 9.00 (d, J=18.5 Hz, 1H), 8.23 (q, J=10.0, 8.6 Hz, 2H), 8.16 (s, 1H), 8.10 (d, J=8.2 Hz, 1H), 7.84 (dt, J=49.1, 7.5 Hz, 3H), 7.62 (s, 1H), 4.70 (s, 2H), 3.80 (d, J=5.8 Hz, 1H), 2.88 (dd, J=10.2, 5.8 Hz, 2H), 2.64-2.58 (m, 2H), 1.93-1.81 (m, 4H), 1.54 (q, J=7.8 Hz, 2H), 1.31 (q, J=7.5 Hz, 2H), 0.90 (dt, J=7.4, 3.7 Hz, 2H), 0.83 (t, J=7.4 Hz, 3H).

LRMS (ESI+) m/z calcd for C₂₃H₃₀N₅O₃S⁺ [M+H]⁺ 456.2. found 456.2.

### Synthesis of N-(1-(butyl sulfonyl) piperidin-4-yl)-N-(oxazol-4-ylmethyl) isoquinoline-3-carboxamide (78)

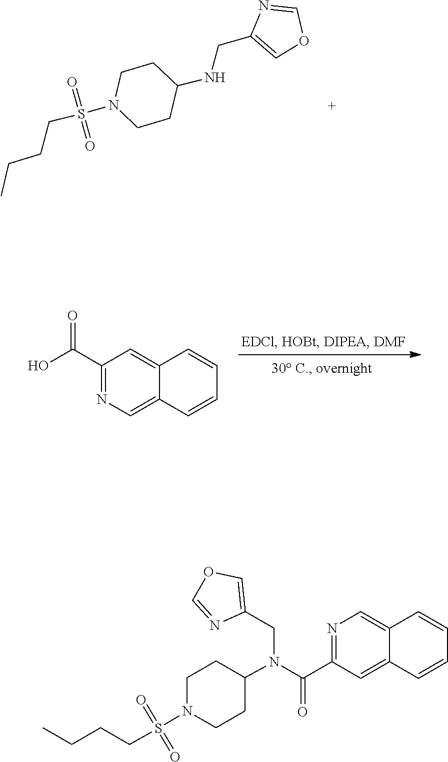 [SMILES: CCCCS(=O)(=O)N1CCC(NCc2cocn2)CC1 ; O=C(O)c1cc2ccccc2cn1 ; CCCCS(=O)(=O)N1CCC(N(Cc2cocn2)C(=O)c2cc3ccccc3cn2)CC1]

Yield 54%, White/light yellow solid, ¹H NMR (500 MHz, Chloroform-d) δ 9.20 (s, 1H), 8.09 (s, 1H), 8.05-7.65 (m, 5H), 7.44 (s, 1H), 4.72-4.62 (m, 2.5H), 4.07 (s, 0.5H), 3.88 (dd, J=47.9, 12.2 Hz, 2H), 3.02-2.78 (m, 3H), 2.63-2.52 (m, 1H), 2.06-1.97 (m, 4H), 1.86-1.69 (m, 2H), 1.51-1.34 (m, 2H), 1.01-0.86 (m, 3H).

LRMS (ESI+) m/z calcd for C₂₃H₂₉N₄O₄S⁺ [M+H]⁺ 457.2. found 457.6.

### Synthesis of N-(1-(butyl sulfonyl) piperidin-4-yl)-N-(thiazol-4-yl methyl) isoquinoline-3-carboxamide (79)

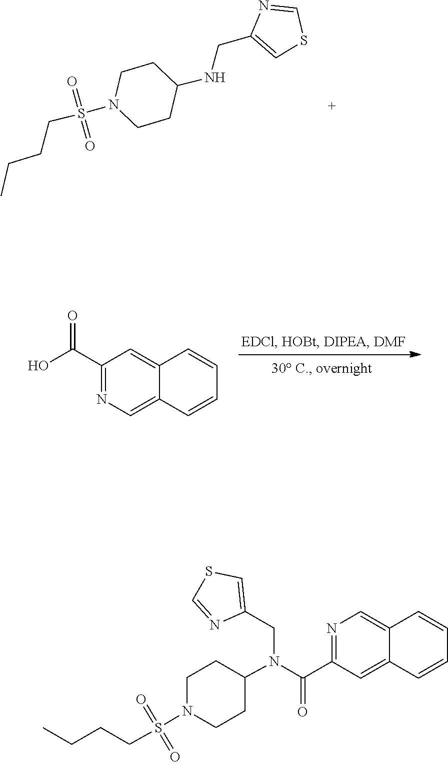 [SMILES: CCCCS(=O)(=O)N1CCC(NCc2cscn2)CC1 ; O=C(O)c1cc2ccccc2cn1 ; CCCCS(=O)(=O)N1CCC(N(Cc2cscn2)C(=O)c2cc3ccccc3cn2)CC1]

Yield 53%, White/light yellow solid, H NMR (500 MHz, Chloroform-d) δ 9.19 (d, J=25.2 Hz, 1H), 8.83-8.58 (m, 1H), 8.18-7.58 (m, 5H), 7.47 (s, 0.5H), 7.15 (s, 0.5H), 4.93 (s, 2H), 4.68 (s, 0.5H), 4.10 (s, 0.5H), 3.85 (dd, J=49.6, 12.3 Hz, 2H), 2.93-2.56 (m, 3H), 2.56 (s, 1H), 2.09-1.66 (m, 6H), 1.50-1.35 (m, 2H), 0.97-0.90 (m, 3H). LRMS (ESI+) m/z calcd for C₂₃H₂₉N₄O₃S₂⁻ [M+H]⁺ 473.2. found 473.6.

### Synthesis of N-(1-(butyl sulfonyl) piperidin-4-yl)-N-(thiazol-2-yl methyl) isoquinoline-3-carboxamide (80)

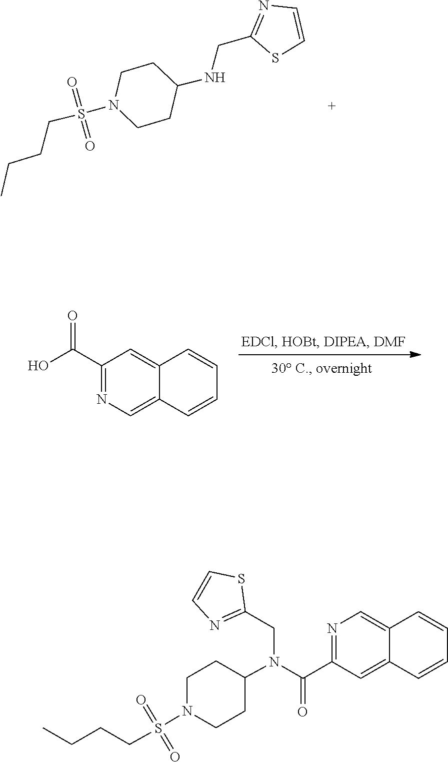 [SMILES: CCCCS(=O)(=O)N1CCC(NCc2nccs2)CC1 ; O=C(O)c1cc2ccccc2cn1 ; CCCCS(=O)(=O)N1CCC(N(Cc2nccs2)C(=O)c2cc3ccccc3cn2)CC1]

Yield 9%, White solid, ¹H NMR (500 MHz, Chloroform-d) δ 9.21 (d, J=15.3 Hz, 1H), 8.20 (s, 1H), 8.20-7.89 (m, 2H), 7.83-7.61 (m, 3H), 7.35-7.24 (m, 1H), 5.11 (d, J=48.5 Hz, 2H), 4.62 (s, 0.5H), 4.20 (0.5H), 3.86 (dd, J=46.1, 12.3 Hz, 2H), 3.00-2.75 (m, 3H), 2.58 (s, 1H), 1.98-1.92 (m, 4H), 1.80-1.74 (m, 2H), 1.47-1.40 (m, 2H), 0.97-0.91 (m, 3H).

LRMS (ESI+) m/z calcd for C₂₃H₂₉N₄O₃S₂⁺ [M+H]⁺ 473.2. found 473.6.

### Synthesis of N-(1-(butyl sulfonyl) piperidin-4-yl)-N-(thiazol-5-yl methyl) isoquinoline-3-carboxamide (81)

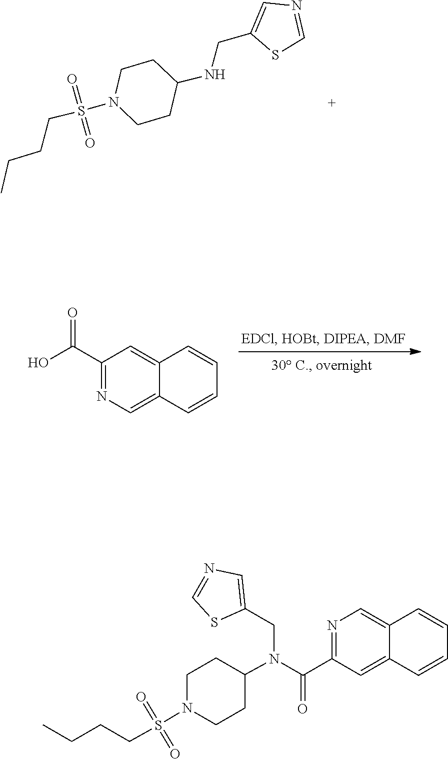 [SMILES: CCCCS(=O)(=O)N1CCC(NCc2cncs2)CC1 ; O=C(O)c1cc2ccccc2cn1 ; CCCCS(=O)(=O)N1CCC(N(Cc2cncs2)C(=O)c2cc3ccccc3cn2)CC1]

Yield 6%, Yellow oil, ¹H NMR (500 MHz, Chloroform-d) δ 9.20 (s, 1H), 8.77-8.66 (m, 1H), 8.16 (s, 1H), 8.07-7.59 (m, 5H), 4.97 (d, J=66.9 Hz, 2H), 4.56-4.52 (m, 0.5H), 4.19 (s, 1H), 3.97-3.80 (m, 3H), 2.98-2.76 (m, 3H), 2.57 (t, J=11.7 Hz, 1H), 2.06-1.89 (m, 4H), 1.80-1.70 (m, 2H), 1.47-1.39 (m, 2H), 1.00-0.88 (m, 3H).

LRMS (ESI+) m/z calcd for C₂₃H₂₉N₄O₃S₂⁺ [M+H]⁺ 473.2. found 473.6.

### Synthesis of N-(1-(butylsulfonyl)piperidin-4-yl)-N-(cyclopentylmethyl)isoquinoline-3-carboxamide (82)

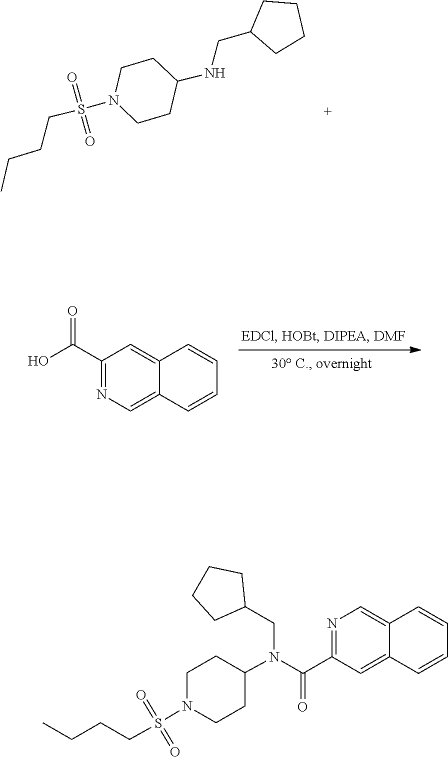 [SMILES: CCCCS(=O)(=O)N1CCC(NCC2CCCC2)CC1 ; O=C(O)c1cc2ccccc2cn1 ; CCCCS(=O)(=O)N1CCC(N(CC2CCCC2)C(=O)c2cc3ccccc3cn2)CC1]

Yield 54%, Yellow oil, ¹H NMR (400 MHz, Chloroform-d) δ 9.20 (s, 1H), 8.05-7.86 (m, 3H), 7.72 (dddd, J=28.2, 8.1, 6.9, 1.2 Hz, 2H), 4.37 (s, 0.5H), 3.97-3.75 (m, 2.5H), 3.54-3.46 (m, 2H), 3.19-2.78 (m, 4H), 2.46 (d, J=46.5 Hz, 1H), 2.19-1.62 (m, 11H), 1.51-1.20 (m, 6H), 0.93 (dd, J=14.0, 7.0 Hz, 5H).

LRMS (ESI+) m/z calcd for C₂₅H₃₆N₃O₃S⁺ [M+H]⁺ 458.2. found 458.7.

### Synthesis of N-(1-(butylsulfonyl)piperidin-4-yl)-N-methylisoquinoline-3-carboxamide (83)

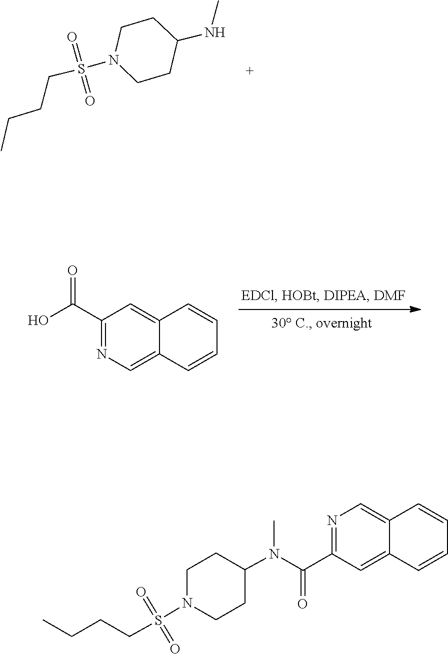 [SMILES: CCCCS(=O)(=O)N1CCC(NC)CC1 ; O=C(O)c1cc2ccccc2cn1 ; CCCCS(=O)(=O)N1CCC(N(C)C(=O)c2cc3ccccc3cn2)CC1]

Yield 97%, White solid, ¹H NMR (500 MHz, Chloroform-d) δ 9.24 (s, 0.6H), 9.20 (s, 0.4H), 8.11-7.98 (m, 2H), 7.91 (m, 1H), 7.73 (dt, J=34.0, 7.3 Hz, 2H), 4.81-4.70 (m, 1H), 4.04-3.81 (m, 2H), 3.10-2.53 (m, 7H), 2.04-1.67 (m, 6H), 1.44 (ddd, J=29.9, 15.2, 7.4 Hz, 2H), 0.94 (dt, J=30.6, 7.3 Hz, 3H).

LRMS (ESI+) m/z calcd for C₂₀H₂₈N₃O₃S⁺ [M+H]⁺ 390.2. found 390.3.

### Synthesis of N-(1-(butylsulfonyl)piperidin-4-yl)-N-ethylisoquinoline-3-carboxamide (84)

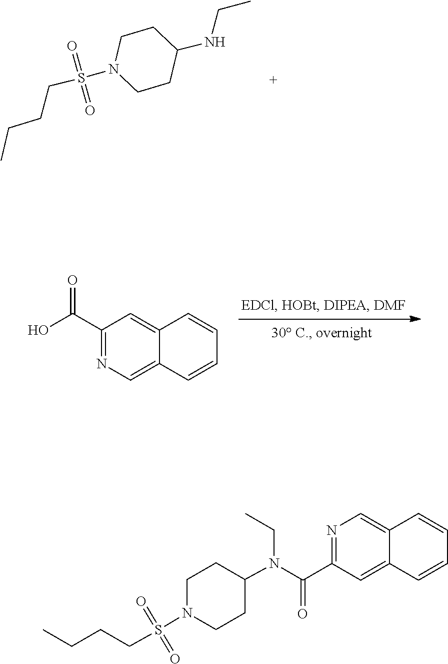 [SMILES: CCCCS(=O)(=O)N1CCC(NCC)CC1 ; O=C(O)c1cc2ccccc2cn1 ; CCCCS(=O)(=O)N1CCC(N(CC)C(=O)c2cc3ccccc3cn2)CC1]

Yield 35%, Yellow oil, ¹H NMR (400 MHz, Chloroform-d) δ 9.21 (s, 1H), 8.06-7.93 (m, 2H), 7.90 (d, J=7.9 Hz, 1H), 7.76 (t, J=7.3 Hz, 1H), 7.69 (t, J=7.5 Hz, 1H), 4.68-4.63 (m, 0.5H), 4.01-3.80 (m, 2.5H), 3.57-3.44 (m, 2H), 3.01-2.79 (m, 3H), 2.60-2.50 (m, 1H), 2.05-1.90 (m, 4H), 1.87-1.69 (m, 2H), 1.50-1.30 (m, 4H), 1.08-0.90 (m, 4H).

LRMS (ESI+) m/z calcd for C₂₁H₃₀N₃O₃S⁺ [M+H]⁺ 404.2. found 404.1.

### Synthesis of N-(1-(butylsulfonyl)piperidin-4-yl)-N-propylisoquinoline-3-carboxamide (85)

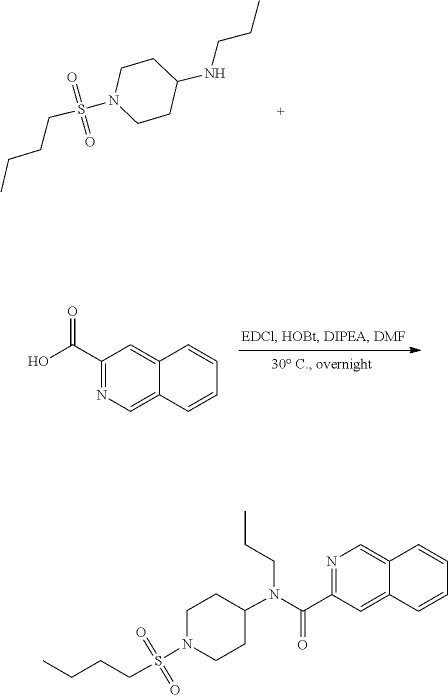 [SMILES: CCCCS(=O)(=O)N1CCC(NCCC)CC1 ; O=C(O)c1cc2ccccc2cn1 ; CCCCS(=O)(=O)N1CCC(N(CCC)C(=O)c2cc3ccccc3cn2)CC1]

Yield 58%, Yellow oil, ¹H NMR (500 MHz, Chloroform-d) δ 9.21 (d, J=7.7 Hz, 1H), 8.07-7.94 (m, 2H), 7.90 (m, 1H), 7.72 (dt, J=35.4, 7.6 Hz, 2H), 4.62-4.58 (m, 0.5H), 4.05-3.78 (m, 2.5H), 3.35 (m, 2H), 3.01-2.87 (m, 2H), 2.82 (t, J=8.0 Hz, 1H), 2.54 (t, J=12.1 Hz, 1H), 2.03-1.69 (m, 7H), 1.54-1.34 (m, 3H), 1.03-0.60 (m, 6H).

LRMS (ESI+) m/z calcd for C₂₂H₃₂N₃O₃S⁺ [M+H]⁺ 418.2. found 418.5.

### Synthesis of N-butyl-N-(1-(butylsulfonyl)piperidin-4-yl)isoquinoline-3-carboxamide (86)

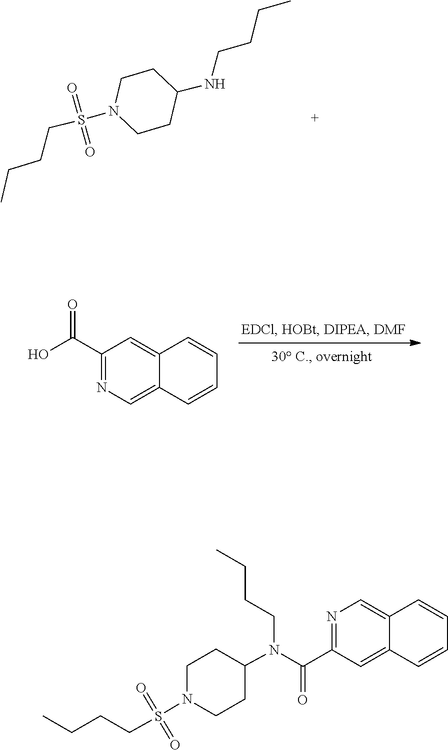

Yield 54%, Yellow oil, ¹H NMR (500 MHz, Chloroform-d) δ 9.20 (d, J=7.8 Hz, 1H), 8.06-7.95 (m, 2H), 7.90 (d, J=7.9 Hz, 1H), 7.72 (dt, J=35.4, 7.3 Hz, 2H), 4.64-4.59 (m, 0.5H), 4.02-3.78 (m, 2.5H), 3.40 (q, J=11.7 Hz, 2H), 2.95 (t, J=7.9 Hz, 2H), 2.87-2.76 (m, 1H), 2.54 (t, J=11.6 Hz, 1H), 2.02-1.66 (m, 7H), 1.42 (m, 4H), 0.97 (tt, J=32.7, 7.2 Hz, 6H), 0.67 (t, J=7.3 Hz, 1H).

LRMS (ESI+) m/z calcd for C₂₃H₃₄N₃O₃S⁺ [M+H]⁺ 432.2. found 432.5.

### Synthesis of N-(1-(butylsulfonyl)piperidin-4-yl)-N-(cyclopropylmethyl)isoquinoline-3-carboxamide (87)

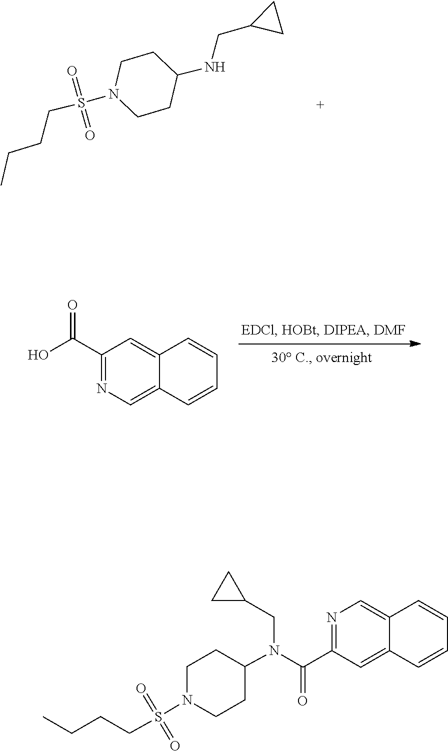 [SMILES: CCCCS(=O)(=O)N1CCC(NCC2CC2)CC1 ; O=C(O)c1cc2ccccc2cn1 ; CCCCS(=O)(=O)N1CCC(N(CC2CC2)C(=O)c2cc3ccccc3cn2)CC1]

Yield 77%, Yellow oil, ¹H NMR (500 MHz, Chloroform-d) δ 9.21 (s, 1H), 8.07-7.86 (m, 3H), 7.73 (dt, J=35.0, 7.2 Hz, 3H), 4.58 (m, 0.5H), 4.04-3.75 (m, 2.5H), 3.39 (d, J=6.6 Hz, 2H), 3.03-2.46 (m, 4H), 2.18-1.65 (m, 6H), 1.51-1.34 (m, 2H), 1.02-0.81 (m, 4H), 0.66-0.52 (m, 1H), 0.47-0.32 (m, 2H), −0.11-−0.21 (m, 1H).

LRMS (ESI+) m/z calcd for C₂₃H₃₂N₃O₃S⁺ [M+H]⁺ 430.2. found 430.7.

### Synthesis of N-(1-(butylsulfonyl)piperidin-4-yl)-N-(3,3,3-trifluoropropyl)isoquinoline-3-carboxamide (88)

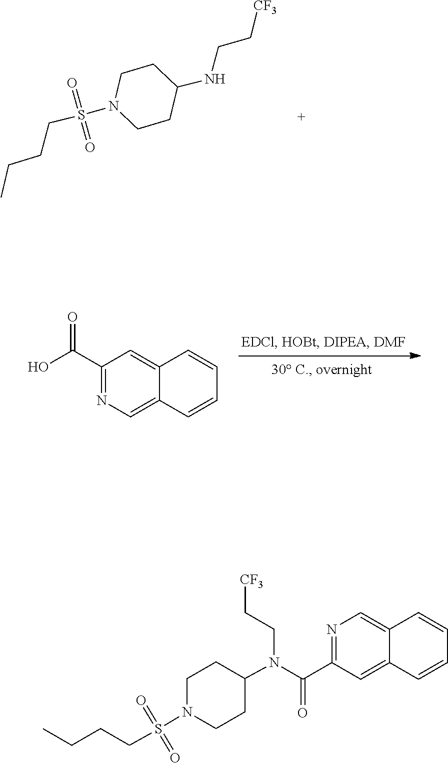 [SMILES: CCCCS(=O)(=O)N1CCC(NCCC(F)(F)F)CC1 ; O=C(O)c1cc2ccccc2cn1 ; CCCCS(=O)(=O)N1CCC(N(CCC(F)(F)F)C(=O)c2cc3ccccc3cn2)CC1]

Yield 53%, Yellow oil, ¹H NMR (400 MHz, Chloroform-d) δ 9.20 (s, 1H), 8.14-8.10 (m, 1H), 8.04 (d, J=8.2 Hz, 1H), 7.92 (d, J=8.1 Hz, 1H), 7.78 (t, J=7.3 Hz, 1H), 7.72 (t, J=7.5 Hz, 1H), 4.64-4.58 (m, 0.4H), 4.15-3.83 (m, 2.6H), 3.72-3.62 (m, 2H), 3.00-2.81 (m, 3H), 2.73-2.55 (m, 3H), 2.07-1.87 (m, 4H), 1.87-1.70 (m, 2H), 1.53-1.34 (m, 2H), 1.03-0.88 (m, 3H).

LRMS (ESI+) m/z calcd for C₂₂H₂₉F₃N₃O₃S⁺ [M+H]⁻ 472.2. found 472.5.

### Synthesis of N-(1-(butylsulfonyl)piperidin-4-yl)-N-(2-hydroxyethyl)isoquinoline-3-carboxamide (89)

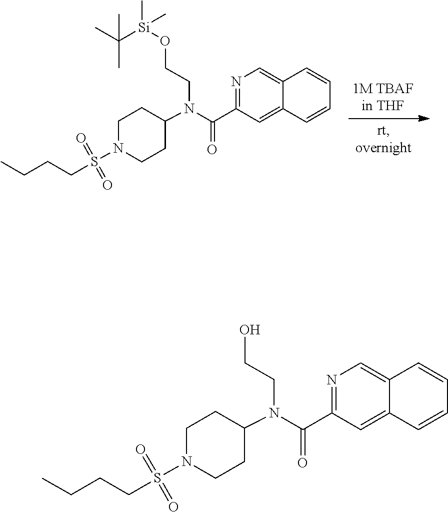 [SMILES: CCCCS(=O)(=O)N1CCC(N(CCO[Si](C)(C)C(C)(C)C)C(=O)c2cc3ccccc3cn2)CC1 ; CCCCS(=O)(=O)N1CCC(N(CCO)C(=O)c2cc3ccccc3cn2)CC1]

Yield 55%, White oily solid, H NMR (400 MHz, Chloroform-d) δ 9.17 (s, 1H), 8.20 (d, J=37.8 Hz, 1H), 7.99 (dd, J=42.8, 8.2 Hz, 2H), 7.84-7.69 (m, 2H), 6.59 (s, 1H), 4.45-4.39 (m, 0.5H), 4.12-3.80 (m, 4.5H), 3.71-3.58 (m, 2H), 3.03-2.80 (m, 3H), 2.63-2.57 (m, 1H), 2.17-2.05 (m, 3H), 1.90-1.72 (m, 3H), 1.52-1.36 (m, 2H), 0.97 (t, J=7.3 Hz, 3H).

LRMS (ESI+) m/z calcd for C₂₁H₃₀N₃O₄S⁺ [M+H]⁺ 420.2. found 420.2.

### Synthesis 2-(N-(1-(butylsulfonyl)piperidin-4-yl)isoquinoline-3-carboxamido)ethyl acetate (90)

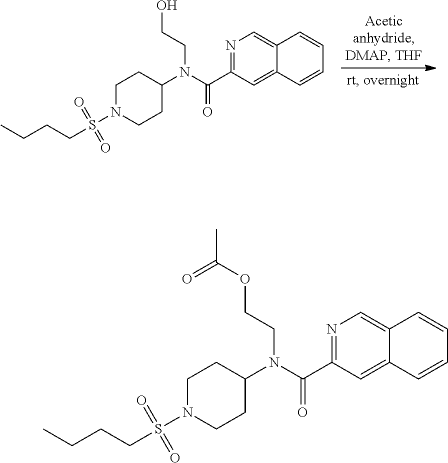 [SMILES: CCCCS(=O)(=O)N1CCC(N(CCO)C(=O)c2cc3ccccc3cn2)CC1 ; CCCCS(=O)(=O)N1CCC(N(CCOC(C)=O)C(=O)c2cc3ccccc3cn2)CC1]

Yield 58%, White oily solid, ¹H NMR (400 MHz, Chloroform-d) δ 9.20 (s, 1H), 8.09-8.01 (m, 2H), 7.91 (d, J=8.2 Hz, 1H), 7.77 (t, J=7.3 Hz, 1H), 7.71 (t, J=7.3 Hz, 1H), 4.63-4.59 (m, 0.4H), 4.39-4.36 (m, 1H), 4.21-3.67 (m, 6H), 3.02-2.79 (m, 3H), 2.62-2.52 (m, 1H), 2.13-1.72 (m, 9H), 1.52-1.37 (m, 2H), 0.97-0.90 (m, 3H).

LRMS (ESI+) m/z calcd for C₂₃H₃₂N₃O₅S⁺ [M+H]⁺ 462.2. found 462.1.

### Synthesis of N-(2-aminoethyl)-N-(1-(butylsulfonyl)piperidin-4-yl)isoquinoline-3-carboxamide (91)

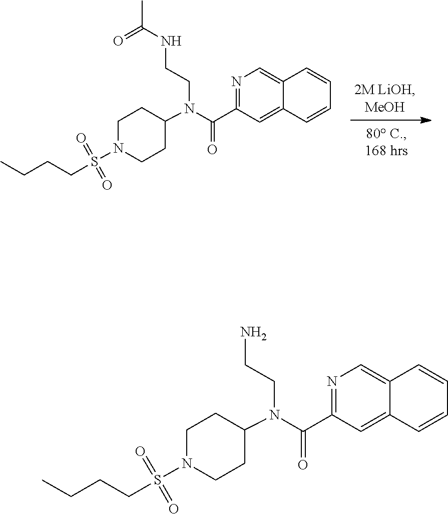 [SMILES: CCCCS(=O)(=O)N1CCC(N(CCNC(C)=O)C(=O)c2cc3ccccc3cn2)CC1 ; CCCCS(=O)(=O)N1CCC(N(CCN)C(=O)c2cc3ccccc3cn2)CC1]

Yield 18%, Yellow oil, ¹H NMR (400 MHz, DMSO-d₆) δ 9.39 (s, 1H), 9.00 (s, 1H), 8.57 (s, 1H), 8.25 (d, J=8.2 Hz, 1H), 8.20 (d, J=7.7 Hz, 1H), 7.88 (ddd, J=8.2, 6.9, 1.3 Hz, 1H), 7.81 (ddd, J=8.1, 6.9, 1.2 Hz, 1H), 3.58-3.43 (m, 4H), 3.03-2.93 (m, 2H), 2.86 (td, J=12.3, 2.6 Hz, 4H), 1.95-1.85 (m, 2H), 1.67-1.55 (m, 2H), 1.38 (dq, J=14.6, 7.4 Hz, 4H), 0.88 (t, J=7.3 Hz, 3H).

LRMS (ESI+) m/z calcd for C₂₁H₃₁N₄O₃S⁺ [M+H]⁺ 419.2. found 419.3.

### Synthesis of N-(2-acetamidoethyl)-N-(1-(butylsulfonyl)piperidin-4-yl)isoquinoline-3-carboxamide (92)

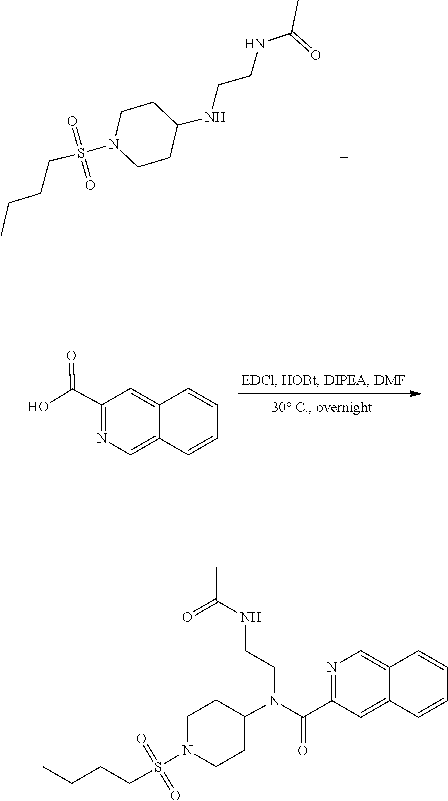 [SMILES: CCCCS(=O)(=O)N1CCC(NCCNC(C)=O)CC1 ; O=C(O)c1cc2ccccc2cn1 ; CCCCS(=O)(=O)N1CCC(N(CCNC(C)=O)C(=O)c2cc3ccccc3cn2)CC1]

Yield 18%, Yellow oil, H NMR (400 MHz, Chloroform-d) δ 9.21 (s, 1H), 8.40 (s, 0.5H), 8.18-8.02 (m, 2H), 7.93 (d, J=8.1 Hz, 1H), 7.84-7.77 (m, 1H), 7.76-7.70 (m, 1H), 6.76 (s, 0.5H), 4.25-4.20 (m, 0.5H), 4.01-3.78 (m, 2.5H), 3.70-3.45 (m, 4H), 3.01-2.80 (m, 3H), 2.56 (t, J=11.7 Hz, 1H), 2.30-2.15 (m, 1H), 2.06-1.70 (m, 8H), 1.52-1.35 (m, 2H), 1.01-0.87 (m, 3H).

LRMS (ESI+) m/z calcd for C₂₃H₃₃N₄O₄S⁺ [M+H]⁺ 461.2. found 461.5.

### Synthesis of N-(1-(butylsulfonyl)piperidin-4-yl)-N-(1-cyclopropylethyl)isoquinoline-3-carboxamide (93)

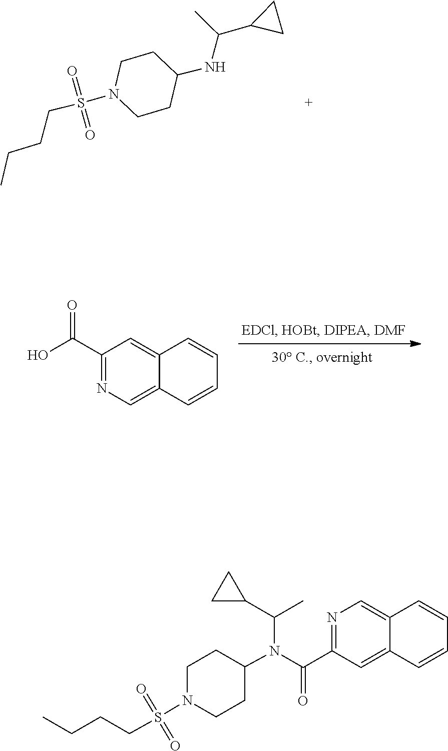 [SMILES: CCCCS(=O)(=O)N1CCC(NC(C)C2CC2)CC1 ; O=C(O)c1cc2ccccc2cn1 ; CCCCS(=O)(=O)N1CCC(N(C(=O)c2cc3ccccc3cn2)C(C)C2CC2)CC1]

Yield 7.5%, White oily solid, ¹H NMR (400 MHz, Chloroform-d) δ 9.18 (s, 1H), 7.93 (dd, J=52.8, 7.8 Hz, 3H), 7.70 (dt, J=30.9, 7.4 Hz, 2H), 4.06-3.31 (m, 3H), 3.14-2.70 (m, 6H), 2.62-2.39 (m, 1H), 2.18-1.61 (m, 6H), 1.52-1.28 (m, 4H), 1.02-0.85 (m, 4H), 0.67-0.25 (m, 3H).

LRMS (ESI+) m/z calcd for C₂₄H₃₄N₃O₃S⁺ [M+H]⁺ 444.2. found 444.4.

### Synthesis of N-(1-(butylsulfonyl)piperidin-4-yl)-N-(cyclobutylmethyl)isoquinoline-3-carboxamide (94)

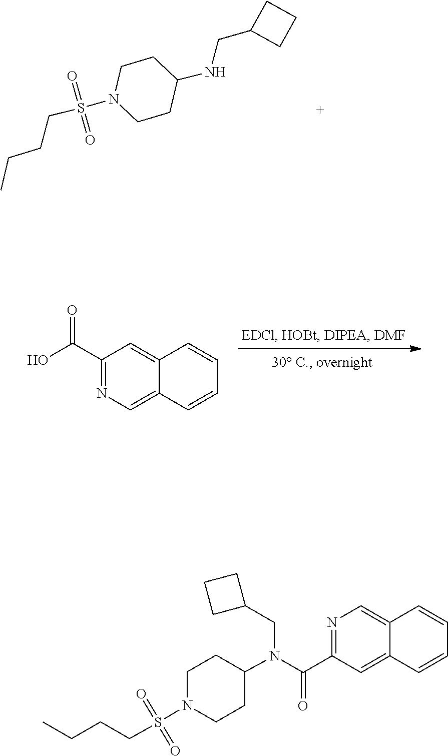 [SMILES: CCCCS(=O)(=O)N1CCC(NCC2CCC2)CC1 ; O=C(O)c1cc2ccccc2cn1 ; CCCCS(=O)(=O)N1CCC(N(CC2CCC2)C(=O)c2cc3ccccc3cn2)CC1]

Yield 41%, White solid, ¹H NMR (600 MHz, Chloroform-d) δ 9.22 (s, 1H), 8.03-7.89 (m, 3H), 7.79-7.66 (m, 2H), 4.45-4.43 (m, 0.5H), 4.03-3.79 (m, 2.5H), 3.59-3.45 (m, 2H), 3.01-2.36 (m, 5H), 2.14-1.65 (m, 1OH), 1.51-1.28 (m, 4H), 0.97-0.91 (m, 3H).

LRMS (ESI+) m/z calcd for C₂₄H₃₄N₃O₃S⁺ [M+H]⁺ 444.2. found 444.0.

### Synthesis of N-(1-(butylsulfonyl)piperidin-4-yl)-N-(oxetan-3-ylmethyl)isoquinoline-3-carboxamide (95)

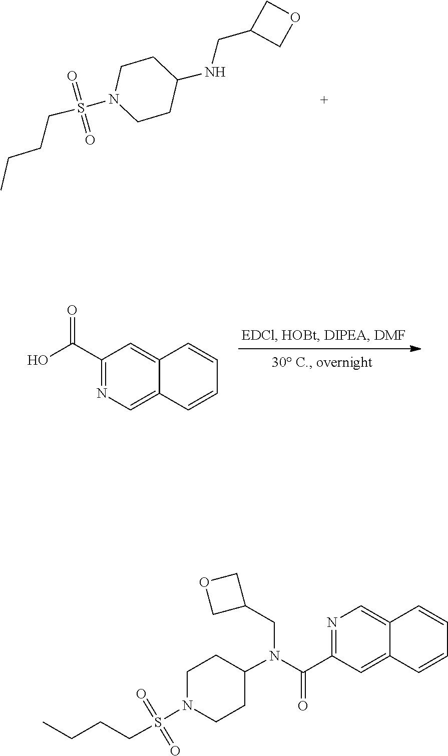 [SMILES: CCCCS(=O)(=O)N1CCC(NCC2COC2)CC1 ; O=C(O)c1cc2ccccc2cn1 ; CCCCS(=O)(=O)N1CCC(N(CC2COC2)C(=O)c2cc3ccccc3cn2)CC1]

Yield 33%, White solid, H NMR (400 MHz, Chloroform-d) δ 9.19 (s, 1H), 8.06 (s, 1H), 8.04-7.87 (m, 2H), 7.81-7.67 (m, 2H), 4.89-4.58 (m, 3.4H), 4.19-3.68 (m, 5.6H), 3.41-3.22 (m, 1H), 2.99-2.52 (m, 4H), 2.06-1.68 (m, 6H), 1.45-1.40 (m, 2H), 1.04-0.83 (m, 3H).

LRMS (ESI+) m/z calcd for C₂₃H₃₂N₃O₄S⁺ [M+H]⁺ 446.2. found 447.4.

### Synthesis of N-(1-(butyl sulfonyl) piperidin-4-yl)-N-(oxetan-2-yl methyl) isoquinoline-3-carboxamide (96)

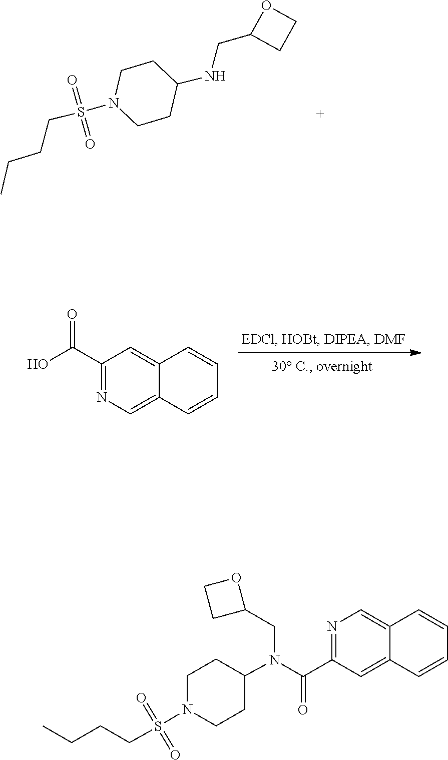 [SMILES: CCCCS(=O)(=O)N1CCC(NCC2CCO2)CC1 ; O=C(O)c1cc2ccccc2cn1 ; CCCCS(=O)(=O)N1CCC(N(CC2CCO2)C(=O)c2cc3ccccc3cn2)CC1]

Yield 20%, Yellow oil, ¹H NMR (500 MHz, Chloroform-d) δ 9.19 (s, 1H), 8.12-7.97 (m, 2H), 7.95-7.85 (m, 1H), 7.73 (dt, J=33.3, 7.4 Hz, 2H), 5.20 (s, 0.6H), 4.90 (s, 0.4H), 4.71-4.46 (m, 2H), 4.22 (s, 0.5H), 4.00-3.78 (m, 4H), 3.56-3.46 (m, 0.5H), 2.98-2.77 (m, 3H), 2.60-2.51 (m, 2H), 2.17-1.93 (m, 5H), 1.84-1.69 (m, 2H), 1.49-1.37 (m, 2H), 1.01-0.85 (m, 3H).

LRMS (ESI+) m/z calcd for C₂₃H₃₂N₃O₄S⁺ [M+H]⁺ 446.2. found 446.6.

### Synthesis of tert-butyl 3-((N-(1-(butylsulfonyl)piperidin-4-yl)isoquinoline-3-carboxamido)methyl)azetidine-1-carboxylate (97)

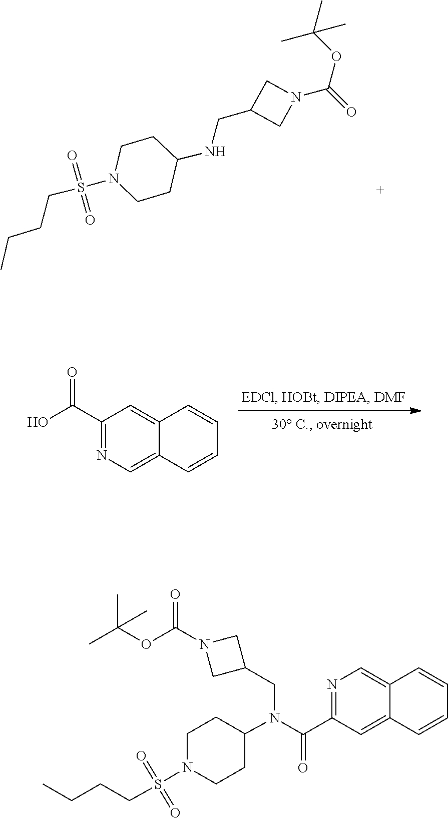 [SMILES: CCCCS(=O)(=O)N1CCC(NCC2CN(C(=O)OC(C)(C)C)C2)CC1 ; O=C(O)c1cc2ccccc2cn1 ; CCCCS(=O)(=O)N1CCC(N(CC2CN(C(=O)OC(C)(C)C)C2)C(=O)c2cc3ccccc3cn2)CC1]

Yield 33%, White solid, ¹H NMR (400 MHz, Chloroform-d) δ 9.19 (s, 1H), 8.07 (s, 1H), 7.97 (dd, J=45.1, 8.1 Hz, 2H), 7.83-7.66 (m, 2H), 4.56-4.45 (m, 0.3H), 4.12-3.63 (m, 8H), 3.54-3.39 (m, 0.7H), 3.06-2.51 (m, 5H), 2.02-1.69 (m, 6H), 1.50-1.30 (m, 12H), 1.01-0.88 (m, 3H).

LRMS (ESI+) m/z calcd for C₂₈H₄₁N₄O₅S⁺ [M+H]⁺ 545.3. found 545.5.

### Synthesis of N-(1-(butylsulfonyl)piperidin-4-yl)-N-(2-methoxyethyl)isoquinoline-3-carboxamide (98)

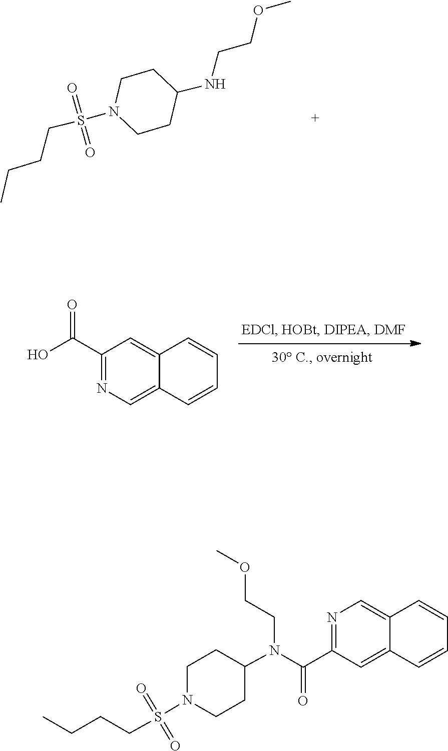

Yield 73%, White solid, ¹H NMR (400 MHz, Chloroform-d) δ 9.20 (s, 1H), 7.96 (dd, J=47.5, 8.2 Hz, 3H), 7.80-7.66 (m, 2H), 4.56-4.49 (m, 0.5H), 4.02-3.34 (m, 8.5H), 3.26-3.10 (m, 1H), 3.02-2.77 (m, 3H), 2.62-2.48 (m, 1H), 2.12-1.70 (m, 6H), 1.50-1.36 (m, 2H), 0.97-0.91 (m, 3H). LRMS (ESI+) m/z calcd for C₂₂H₃₂N₃O₄S⁺ [M+H]⁺ 434.2. found 434.6.

### Synthesis of N-benzyl-N-(1-(sec-butylsulfonyl)piperidin-4-yl)isoquinoline-3-carboxamide (101)

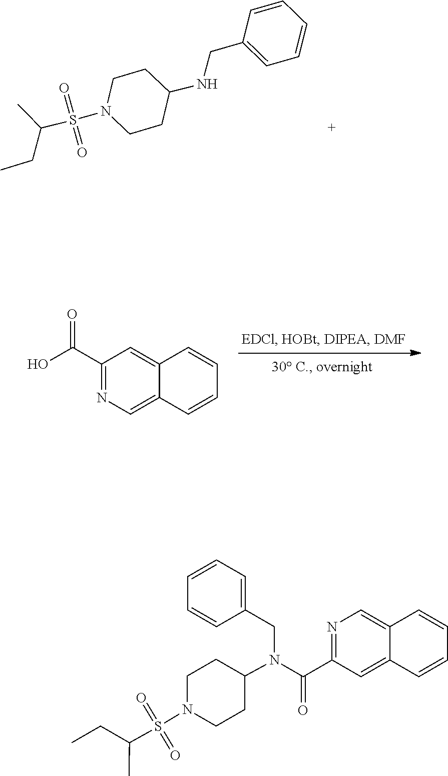 [SMILES: CCC(C)S(=O)(=O)N1CCC(NCc2ccccc2)CC1 ; O=C(O)c1cc2ccccc2cn1 ; CCC(C)S(=O)(=O)N1CCC(N(Cc2ccccc2)C(=O)c2cc3ccccc3cn2)CC1]

Yield 17%, White solid, ¹H NMR (600 MHz, Chloroform-d) δ 9.24 (s, 0.5H), 9.14 (s, 0.5H), 8.19-7.59 (m, 5H), 7.42-7.31 (m, 2H), 7.24-7.14 (m, 3H), 4.81 (s, 2H), 4.67-4.63 (m, 0.5H), 4.15-4.11 (m, 0.5H), 3.92-3.72 (m, 2H), 3.05-2.80 (m, 2H), 2.68-2.57 (m, 1H), 1.99-1.76 (m, SH), 1.52-1.41 (m, 1H), 1.33-1.18 (m, 3H), 0.98 (m, 3H).

LRMS (ESI+) m/z calcd for C₂₆H₃₂N₃O₃S⁺ [M+H]⁺ 466.2. found 466.1.

### Synthesis of N-benzyl-N-(1-(pentan-2-ylsulfonyl)piperidin-4-yl)isoquinoline-3-carboxamide (102)

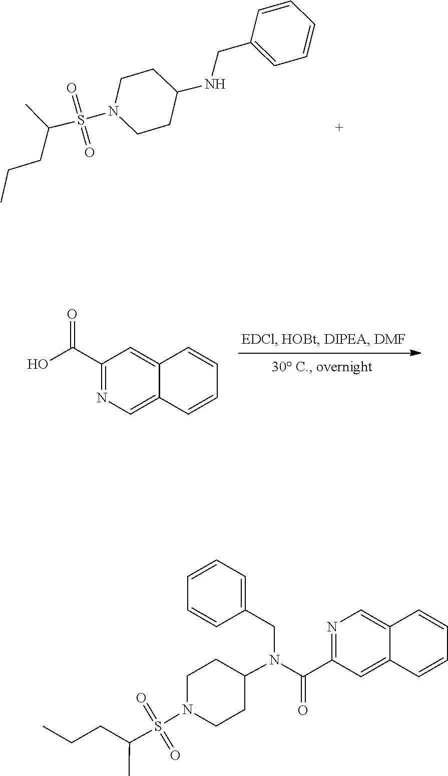 [SMILES: CCCC(C)S(=O)(=O)N1CCC(NCc2ccccc2)CC1 ; O=C(O)c1cc2ccccc2cn1 ; CCCC(C)S(=O)(=O)N1CCC(N(Cc2ccccc2)C(=O)c2cc3ccccc3cn2)CC1]

Yield 6%, White solid, ¹H NMR (400 MHz, Chloroform-d) δ 9.24 (s, 0.5H), 9.19 (s, J=0.5H), 8.17-7.56 (m, 5H), 7.45-7.29 (m, 2H), 7.24-7.14 (m, 3H), 4.81 (s, 2H), 4.70-4.59 (m, 0.5H), 4.16-4.11 (m, 0.5H), 3.83 (dd, J=43.9, 11.5 Hz, 2H), 3.08-2.82 (m, 2H), 2.70-2.54 (m, 1H), 2.00-1.72 (m, 5H), 1.48-1.19 (m, 6H), 0.95-0.88 (m, 3H).

LRMS (ESI+) m/z calcd for C₂₇H₃₄N₃O₃S⁺ [M+H]⁺ 480.2. found 480.3.

### Synthesis of N-benzyl-N-(1-((1-methylcyclopropyl)sulfonyl)piperidin-4-yl)isoquinoline-3-carboxamide (103)

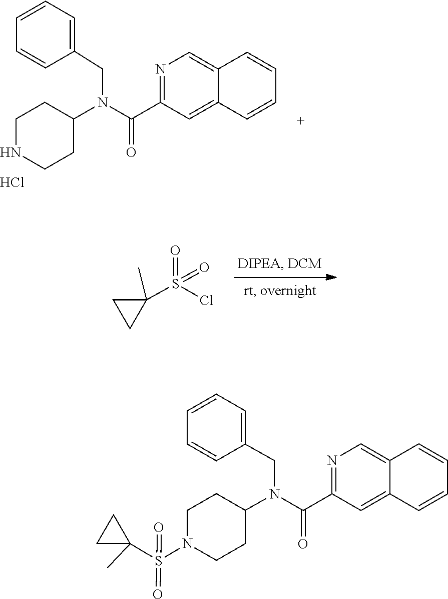

Yield 38%, White solid, ¹H NMR (400 MHz, Chloroform-) 6 9.31-9.07 (m, 1H), 8.18-7.61 (m, 5H), 7.46-7.14 (m, 5H), 4.81 (s, 2H), 4.72-4.60 (m, 0.5H), 4.20-4.07 (m, 0.5H), 3.98-3.73 (m, 2H), 3.10-2.93 (m, 1H), 2.74-2.56 (m, 1H), 1.98-1.75 (m, 4H), 1.44-1.21 (m, 5H), 0.71 (d, J=21.1 Hz, 2H).

LRMS (ESI+) m/z calcd for C₂₆H₃₀N₃O₃S⁺ [M+H]⁺ 464.2. found 464.1.

### Synthesis of N-benzyl-N-(1-(but-3-yn-1-ylsulfonyl)piperidin-4-yl)isoquinoline-3-carboxamide (104)

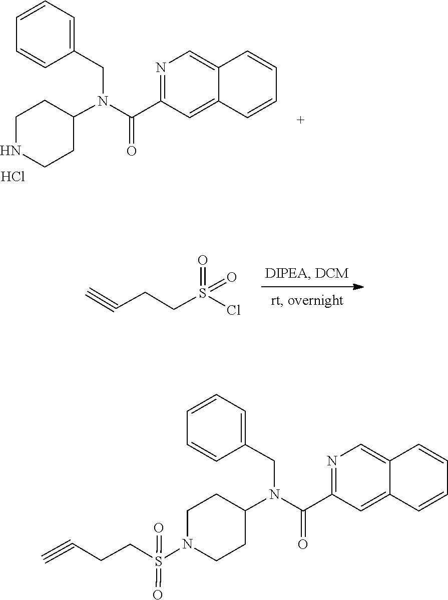

Yield 40%, White solid, ¹H NMR (400 MHz, Chloroform-d) δ 9.29-9.08 (m, 1H), 8.22-7.61 (m, 5H), 7.44-7.12 (m, 5H), 4.82 (s, 2H), 4.67-4.61 (m, 0.5H), 4.20-4.07 (m, 0.5H), 3.81 (dd, J=42.0, 13.1 Hz, 2H), 3.17-2.85 (m, 3H), 2.74-2.53 (m, 3H), 2.02-1.78 (m, 5H).

LRMS (ESI+) m/z calcd for C₂₆H₂₈N₃O₃S⁺ [M+H]⁺ 462.2. found 462.6.

### Synthesis of N-benzyl-N-(1-((3,3,3-trifluoropropyl)sulfonyl)piperidin-4-yl)isoquinoline-3-carboxamide (105)

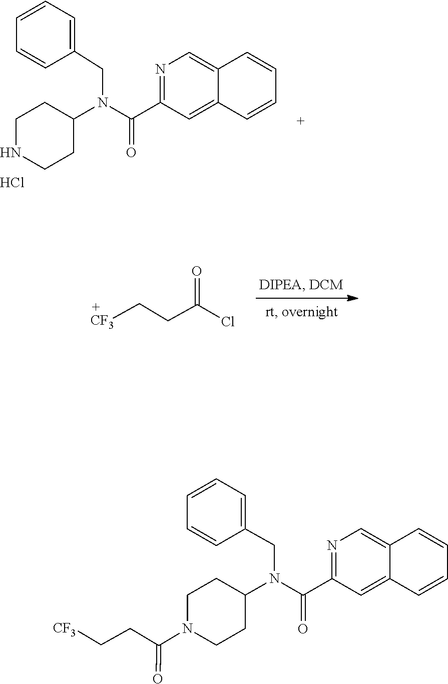 [SMILES: O=C(c1cc2ccccc2cn1)N(Cc1ccccc1)C1CCNCC1 ; Cl ; O=C(Cl)CCC(F)(F)F ; O=C(CCC(F)(F)F)N1CCC(N(Cc2ccccc2)C(=O)c2cc3ccccc3cn2)CC1]

Yield 18%, White solid, ¹H NMR (400 MHz, Chloroform-d) δ 9.30-9.09 (m, 1H), 8.22-7.62 (m, 5H), 7.45-7.15 (m, 5H), 4.82 (s, 2H), 4.62-4.58 (m, 0.5H), 4.19-4.14 (m, 0.5H), 3.94-3.72 (m, 2H), 3.12-2.85 (m, 3H), 2.67-2.46 (m, 3H), 2.05-1.79 (m, 4H).

LRMS (ESI+) m/z calcd for C₂₅H₂₇F₃N₃O₃S⁺ [M+H]⁻ 506.2. found 506.5.

### Synthesis of N-benzyl-N-(1-pentanoylpiperidin-4-yl)isoquinoline-3-carboxamide (106)

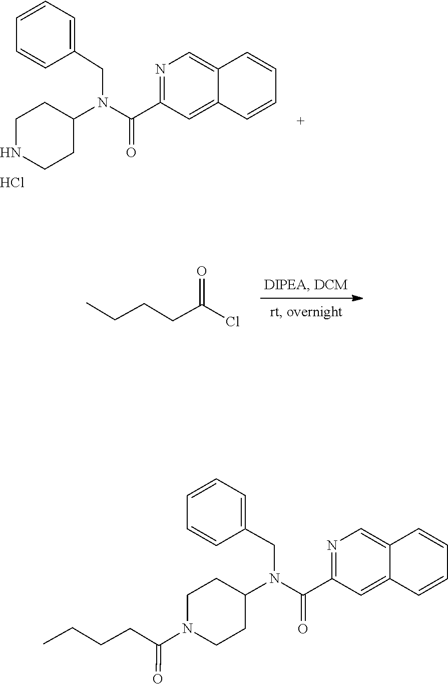

Yield 82%, Yellow oil, H NMR (400 MHz, Chloroform-d) δ 9.20 (d, J=40.8 Hz, 1H), 8.17-7.59 (m, 5H), 7.38-7.32 (m, 2H), 7.21 (s, 3H), 4.88-4.62 (m, 4H), 4.25-4.18 (m, 0.5H), 3.84 (dd, J=35.0, 13.8 Hz, 1H), 3 10 (t, J=13.0 Hz, 0.5H), 2.76 (t, J=13.2 Hz, 0.5H), 2.58 (t, J=13.0 Hz, 0.5H), 2.36-2.13 (m, 2H), 2.01-1.78 (m, 2H), 1.55 (s, 4H), 1.39-1.27 (m, 2H), 0.91 (t, J=7.4 Hz, 3H).

LRMS (ESI+) m/z calcd for C₂₇H₃₂N₃O₂+[M+H]⁺ 430.3. found 430.0.

### Synthesis of N-(1-acetylpiperidin-4-yl)-N-benzylisoquinoline-3-carboxamide (107)

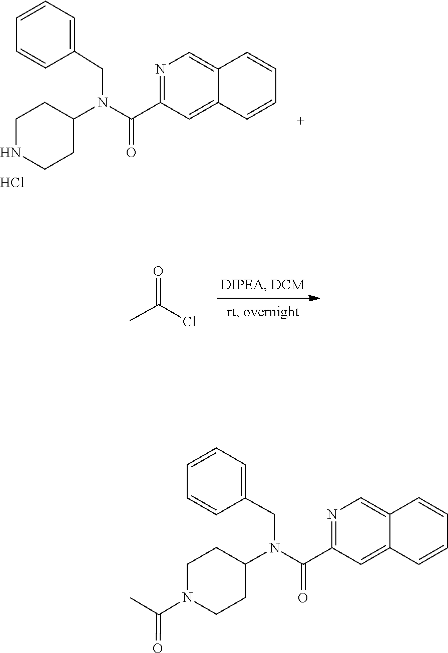

Yield 62%, White solid, ¹H NMR (400 MHz, Chloroform-d) δ 9.30-9.09 (m, 1H), 8.24-7.53 (m, 5H), 7.46-7.11 (m, 5H), 4.93-4.53 (m, 4H), 4.25-4.21 (m, 0.5H), 3.89-3.71 (m, 1H), 3.17-3.10 (m, 0.5H), 2.84-2.77 (m, 0.5H), 2.62-2.56 (m, 0.5H), 2.30-1.61 (m, 7H).

LRMS (ESI+) m/z calcd for C₂₄H₂₆N₃O₂+[M+H]⁺ 388.2. found 388.1.

### Synthesis of N-benzyl-N-(1-(butylsulfonyl)piperidin-4-yl)-5-chloropicolinamide (108)

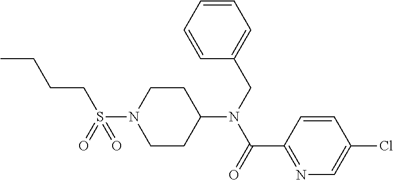 [SMILES: CCCCS(=O)(=O)N1CCC(N(Cc2ccccc2)C(=O)c2ccc(Cl)cn2)CC1]

### Synthesis of N-benzyl-N-(1-(butylsulfonyl)piperidin-4-yl)-5-fluoropicolinamide (109)

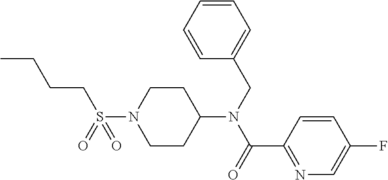 [SMILES: CCCCS(=O)(=O)N1CCC(N(Cc2ccccc2)C(=O)c2ccc(F)cn2)CC1]

### Synthesis of N-benzyl-N-(1-(butylsulfonyl)piperidin-4-yl)-4-methylpicolinamide (110)

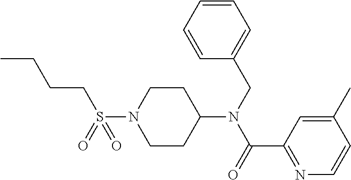 [SMILES: CCCCS(=O)(=O)N1CCC(N(Cc2ccccc2)C(=O)c2cc(C)ccn2)CC1]

### Synthesis of N-benzyl-N-(1-(butylsulfonyl)piperidin-4-yl)-7-chloroisoquinoline-3-carboxamide (1H)

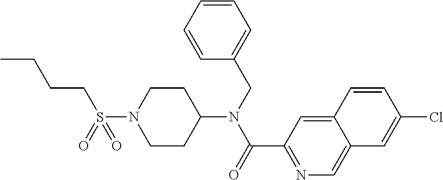 [SMILES: CCCCS(=O)(=O)N1CCC(N(Cc2ccccc2)C(=O)c2cc3ccc(Cl)cc3cn2)CC1]

### Synthesis of N-benzyl-N-(1-(butylsulfonyl)piperidin-4-yl)-5-(trifluoromethyl)picolinamide (112)

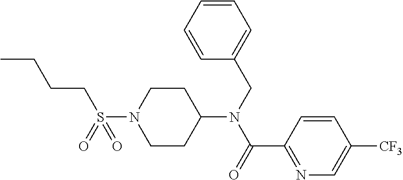 [SMILES: CCCCS(=O)(=O)N1CCC(N(Cc2ccccc2)C(=O)c2ccc(C(F)(F)F)cn2)CC1]

### Synthesis of N-benzyl-N-(1-(butylsulfonyl)piperidin-4-yl)-4-methoxypicolinamide (113)

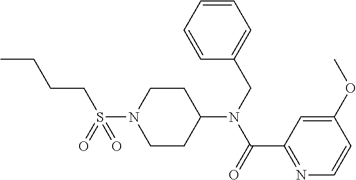 [SMILES: CCCCS(=O)(=O)N1CCC(N(Cc2ccccc2)C(=O)c2cc(OC)ccn2)CC1]

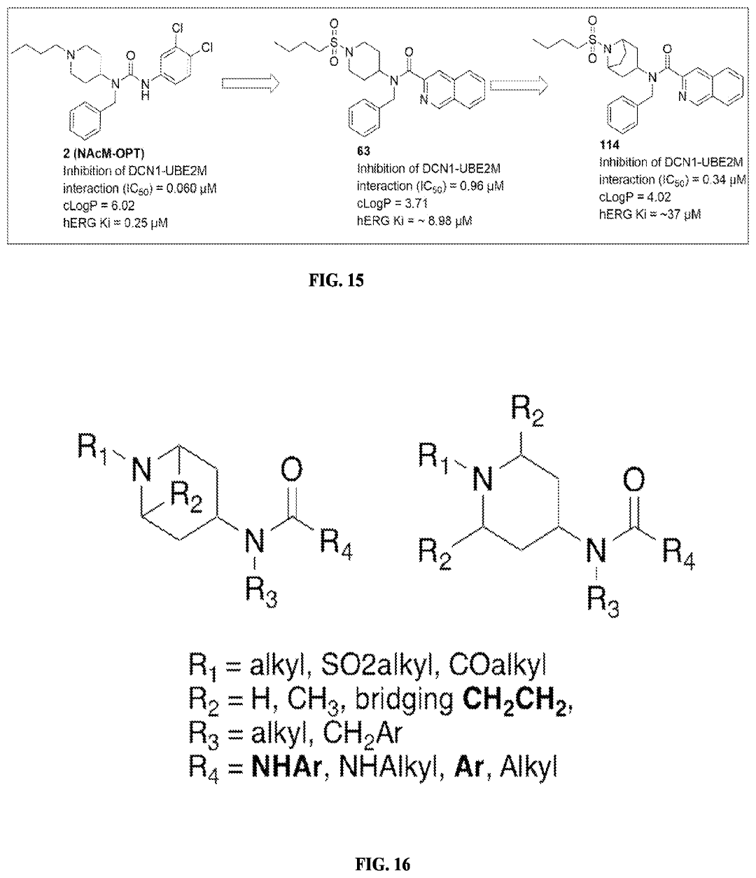

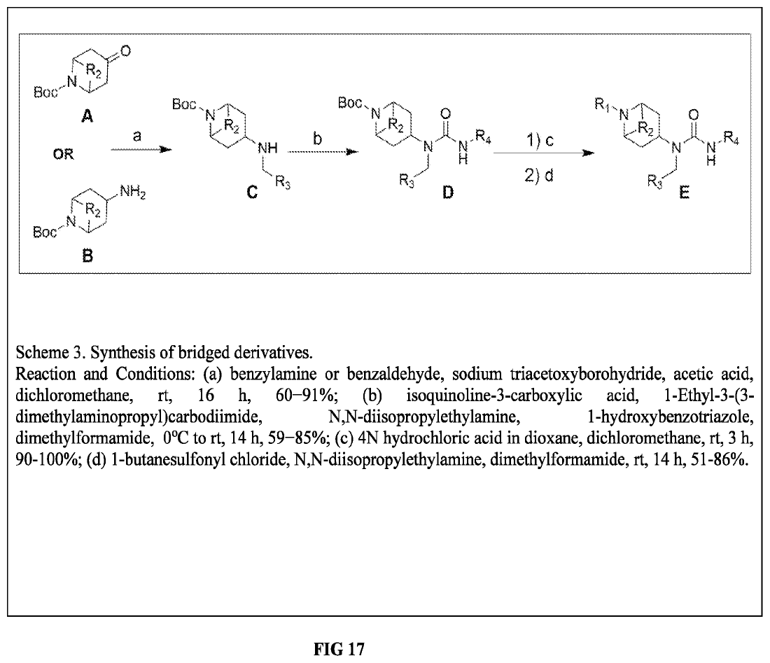

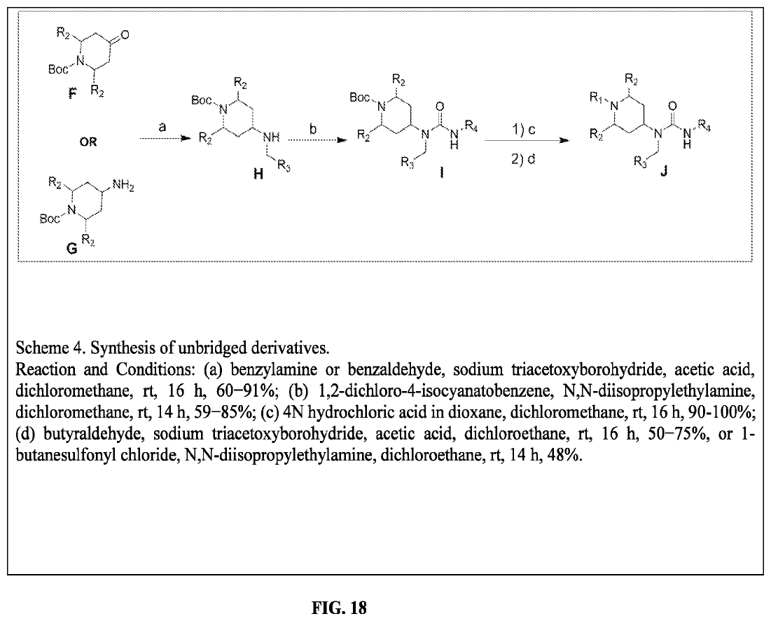

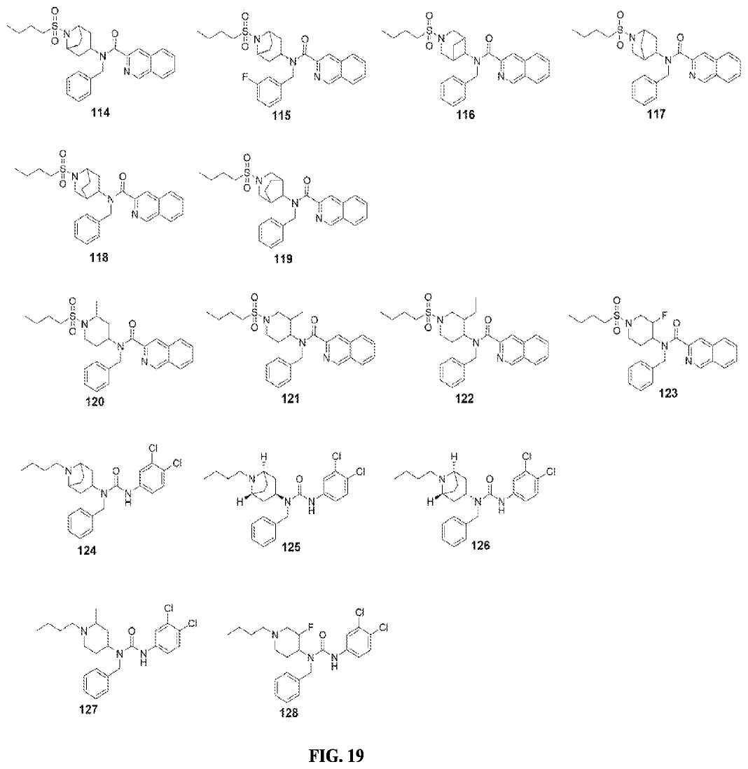

Further consideration was given to potential modifications to/co-structures for piperidinyl urea-based molecules with the goal of improving affinity of DCN for UBE2M, for improving potency, and for reducing hERG affinity. The structural modifications considered are summarized in FIG. 15 and FIG. 16. In particular, further modifications to the Leu Pocket were considered, specifically a bridging group. This is illustrated in FIG. 16. Two synthetic schema were developed for the modifications, as illustrated in FIGS. 17 and 18. FIG. 19 shows representative compounds provided by the described methods.

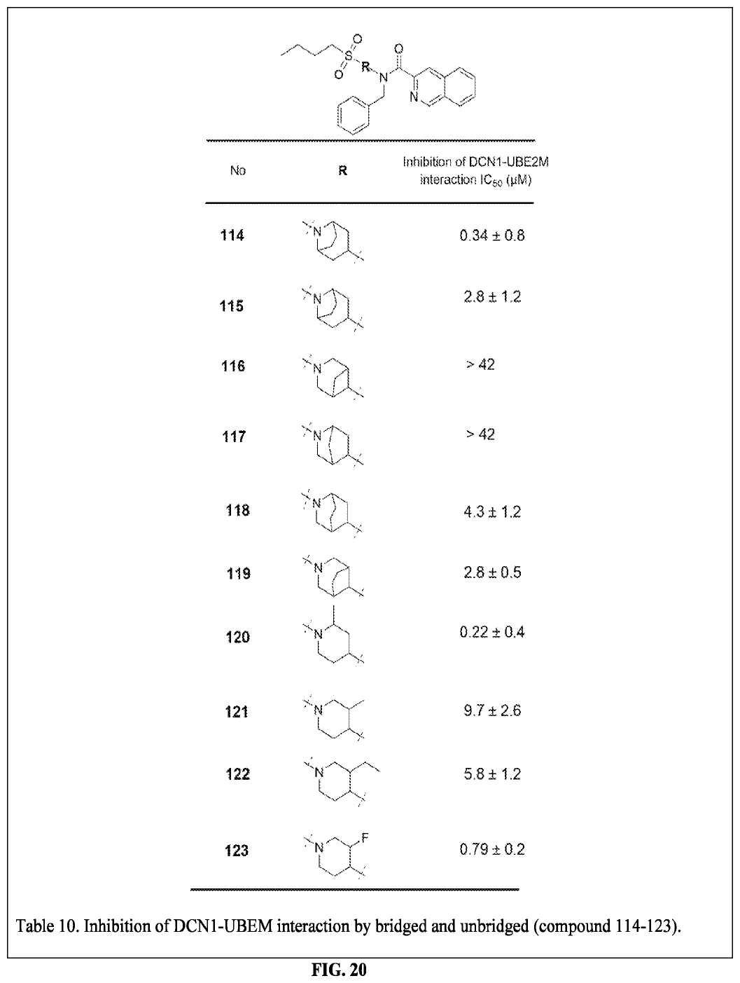

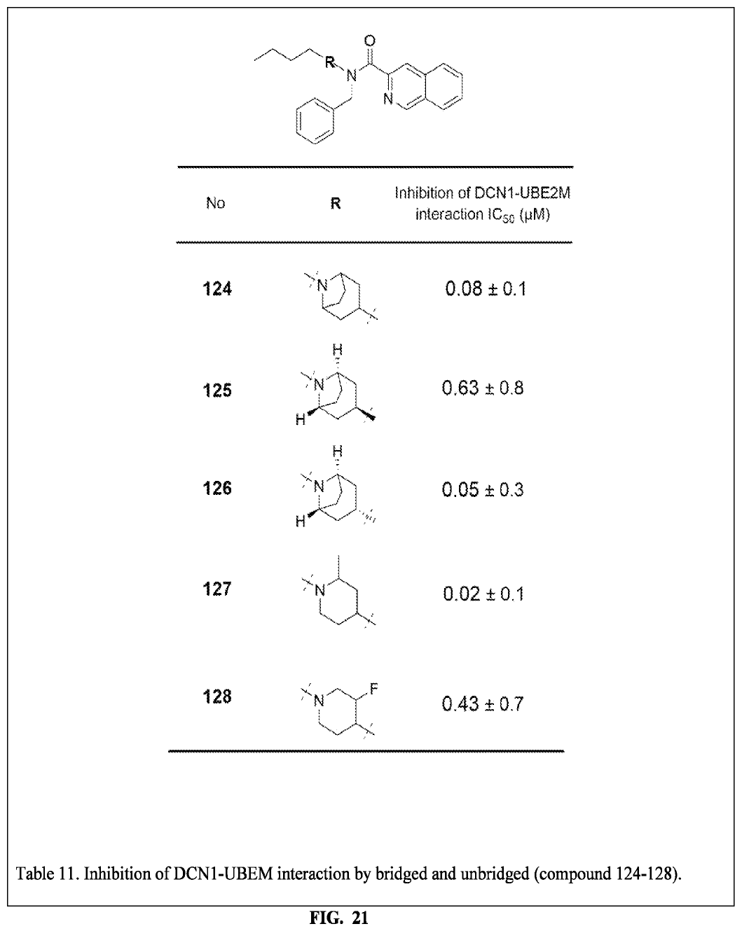

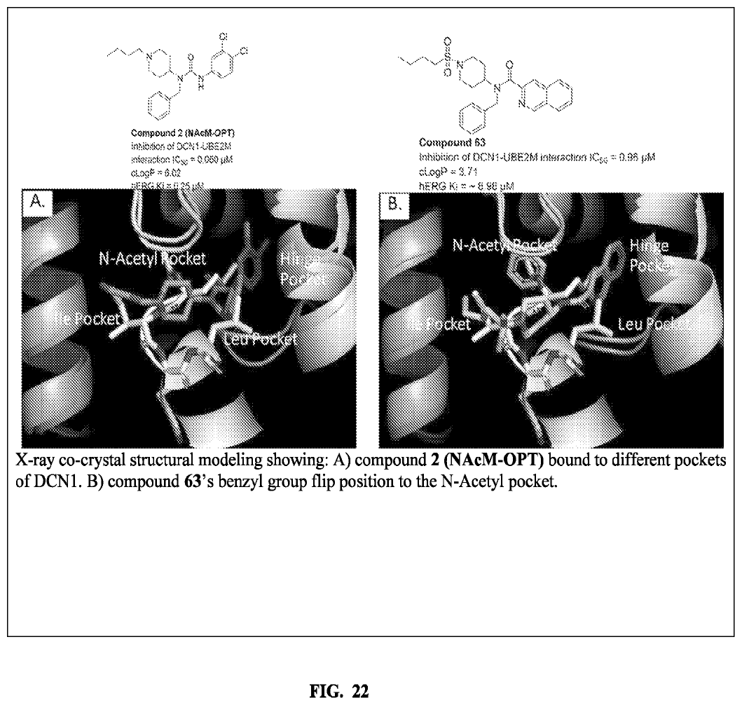

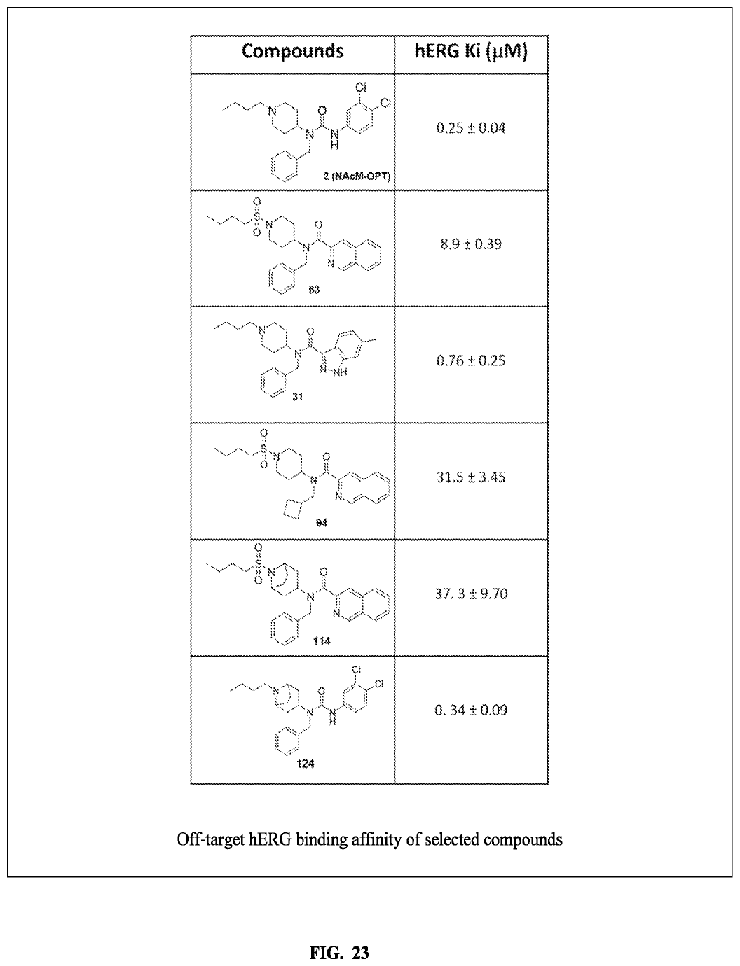

As before, efficacy of the compound modifications evaluated was determined by determining IC₅₀ of molecules modified as shown by TR-FRET. Results are shown in FIGS. 20-22. FIG. 20 shows representative bridged isoquinoline analogue compound formulas provided by the described schema. FIG. 21 shows representative bridged NAcM-OPT analogue compound formulas provided by the described schema.

Target compounds were synthesized by reported methods. Synthesis of the isoquinoline carboxamide derivatives utilized two separate routes (Scheme 3; see FIG. 17). Starting from reductive amination of a boc-protected bridged 4-oxopiperidine (A) or 4-aminopiperidine (B) with benzylamine or benzaldehyde, respectively gave C with yield (60-91%). Subsequent amide coupling with isoquinoline-3-carboxylic acid yielded intermediate D. Finally, acid-mediated deprotection of D and condensation with 1-butanesulfonyl chloride to afford E (Scheme 3).

Synthesis of trisubstituted ureas is outlined in Scheme 4 (see FIG. 18). Reductive amination of a boc-protected bridged 4-oxopiperidine (F) or 4-aminopiperidine (G) with benzylamine or benzaldehyde, respectively afforded benzyl containing amine H, followed by isocyanate coupling yielded intermediate I. Deprotection of I followed by alkylation resulted desired analogues J with yield 48-75%.

General Procedure for Reductive Amination (C). To a solution of boc-protected bridged/unbridged 4-oxopiperidine A/B (0.42 mmol, 1.05 equiv) in dichloromethane (10 ml) was added benzylamine (0.4 mmol, 1 equiv). A few drop of acetic acid was added in the mixture to make the acidic reaction condition. After stirring the mixture for 10 min at room temperature, sodium triacetoxyborohydride (1 mmol, 2.5 equiv) was added slowly. The solution was stirred at room temperature for overnight (ca. 16 h), quenched with a saturated solution of sodium bicarbonate. The mixture was diluted with water, extracted with dichloromethane, dried over magnesium sulfate, filtered, concentrated under reduced pressure. The crude mixture was purified by flash column chromatography (Methanol/Dichloromethane, 1-10%, v/v) to yield the desired product.

General Procedure for Amide Coupling (D). To a solution of Isoquinoline-3-carboxylic acid (0.18 mmol, 1 equiv) in dimethylformamide (1 ml) at 0° C. was added 1-ethyl-3-(3-dimethylaminopropyl)carbodiimide (0.22 mmol, 1.2 equiv), boc-protected amine C (0.18 mmol, 1 equiv), hydroxybenzotriazole (0.22 mmol, 1.2 equiv), and N,N-diisopropylethylamine (0.22 mmol, 1.2 equiv). The reaction mixture was stirred at room temperature for overnight (ca. 16 h), quenched with water, extracted with ethyl acetate, dried over magnesium sulfate, filtered, concentrated under reduced pressure. The crude mixture was purified by flash column chromatography (Methanol/Dichloromethane, 1-10%, v/v).

General Procedure for Boc Deprotection. To a stirred solution of boc-protected intermediate D (0.16 mmol, 1 equiv) in dichloromethane (10 mL) at 0° C. was slowly added 4N hydrochloric acid in dioxane (1.57 mmol, 10 equiv). The reaction mixture was slowly warmed to room temperature, stirred at room temperature for 3 h, and concentrated under reduced pressure. The crude reaction mixture was directly carried onto the next reaction without purification.

General Procedure for Sulfonamide Formation (E). To a stirred solution of the boc-deprotected intermediate (0.091 mmol, 1 equiv) and N,N-diisopropylethylamine (0.2 mmol, 2.2 equiv) in dimethylformamide (1 mL) was added 1-butansulfonyl chloride (0.1 mmol, 1.1 equiv) at 0° C. The mixture was stirred at room temperature overnight. The mixture was added with ethyl acetate, washed with 0.1 N HCl (3×15 mL) and brine (30 mL). The organic layer was dried over magnesium sulfate, filtered and concentrated under reduced pressure. The crude mixture was purified by flash column chromatography (Methanol/Dichloromethane, 4-10%, v/v) to yield the desired product.

General Procedure for Isocyanate Coupling (J). To a stirred solution of boc-protected amine I (0.36 mmol, 1 equiv) in dichloromethane (10 ml) was added 3,4-Dichlorophenyl isocyanate (0.36 mmol, 1 equiv) and N,N-diisopropylethylamine (0.54 mmol, 1.5 equiv). The resulting mixture was stirred at room temperature overnight (ca. 16 h) and concentrated under reduced pressure. The crude mixture was quenched with water, extracted with dichloromethane, dried over magnesium sulfate, filtered, concentrated under reduced pressure. The crude mixture was purified by flash column chromatography (Methanol/Dichloromethane, ₃-9%, v/v) to get the desired product.

Chemistry. ¹H and ¹³C NMR spectra were recorded at room temperature in CDCl₃ on a 400 or 600 MHz Bruker spectrometer. Chemical shifts (6) were recorded in parts per million (ppm) calibrating with internal TMS (3 0.0 ppm for ¹H and ¹³C) or internal CHCl₃ (δ 7.26 ppm for ¹H and 39.5 ppm for ¹³C as the reference. ¹H NMR data are reported as follows: chemical shift, multiplicity (s=singlet, bs=broad singlet, d=doublet, t=triplet, q=quartet, p=pentet, sext=sextet, sep=septet, m=multiplet, dd=double of doublets, dt=doublet of triplets, td=triplet of doublets, qd=quartet of doublets, and coupling constants (J) in hertz (Hz), and integration. Flash column chromatography was performed using a Biotage Isolera One and Biotage KP-SIL SNAP cartridges. All the tested compounds were characterized using 1H NMR and LCMS. All compounds were confirmed to be >95% pure prior to testing. Purity was assessed using an ultraperformance liquid chromatography mass spectrometry (Waters, MA) equipped with a PDA detector and a single quadruple detector. A BEH-C18 column (1.7 μm, 2.1×50 mm²) was used. The flow rate was 0.7 mL/min, and the gradient started with 90% A (0.1% formic acid in H₂O), changed to 95% B (0.1% formic acid in acetonitrile), and then returned to 90% A. The mass spectrometer was operated in the positive-ion mode with electrospray ionization. Integration was performed using Masslynx software 4.2 Starting materials and reagents were obtained from Sigma-Aldrich, TCI, or Alfa Aesar and used without further purification. Thin-layer chromatography and column chromatography were performed using Kieselgel 60 F₂₅₄ (Merck) and silica gel (Kieselgel 60, 230-400 mesh, Merck), respectively.

Specific chemical characterizations of particular compounds according to the foregoing description will now be shown.

### N-benzyl-N-(8-(butylsulfonyl)-8-azabicyclo[3.2.1]octan-3-yl)isoquinoline-3-carboxamide (114)

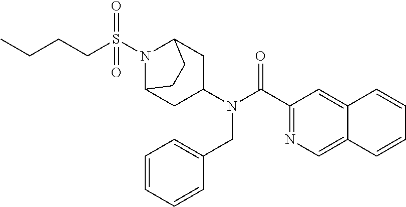 [SMILES: CCCCS(=O)(=O)N1C2CCC1CC(N(Cc1ccccc1)C(=O)c1cc3ccccc3cn1)C2]

Yield 65%, Light-brown solid, ¹H NMR (600 MHz, CDCl₃) δ 9.25-9.12 (m, 1H), 8.19-7.60 (m, 5H), 7.39-7.31 (m, 2H), 7.26-7.09 (m, 3H), 4.76 (s, 2H), 4.29-4.12 (m, 2H), 2.94 (q, J=7.8 Hz, 2H), 2.12-1.94 (m, 3H), 1.92-1.80 (m, 4H), 1.76 (td, J=11.7, 9.8, 6.4 Hz, 2H), 1.65 (s, 1H), 1.44 (q, J=7.5 Hz, 2H), 1.34 (d, J=7.9 Hz, 1H), 0.95 (t, J=7.2 Hz, 3H). ¹³C NMR (151 MHz, CDCl₃) δ 169.8, 151.0, 148.5, 138.8, 136.0, 131.1, 128.5, 128.3, 127.6, 127.4, 126.8, 126.7, 121.6, 56.4, 53.3, 50.8, 45.5, 37.1, 28.8, 25.7, 21.5, 13.7. LRMS (ESI+) m/z calcd for C₂₈H₃₄N₃O₃S⁺ [M+H]⁺ 492.22. found 492.73.

### N-(8-(butylsulfonyl)-8-azabicyclo[3.2.1]octan-3-yl)-N-(3-fluorobenzyl)isoquinoline-3-carboxamide (115)

Yield 58%, Light-brown solid, ¹H NMR (600 MHz, Chloroform-d) δ 9.25-9.10 (m, 1H), 8.20-7.60 (m, 5H), 7.31-7.28 (m, 1H), 7.16-6.80 (m, 3H), 4.79-4.71 (m, 2H), 4.29-4.14 (m, 2H), 3.00-2.91 (m, 2H), 2.14-1.94 (m, 3H), 1.93-1.81 (m, 4H), 1.81-1.68 (m, 3H), 1.48-1.41 (m, 2H), 1.39-1.35 (m, 1H), 0.95 (t, J=7.4 Hz, 3H). ¹³C NMR (151 MHz, CDCl₃) δ 169.8, 163.9, 162.3, 150.9, 148.2, 141.6, 141.5, 136.0, 131.1, 130.0, 130.0, 128.8, 128.7, 127.6, 127.5, 122.2, 121.9, 113.8, 113.7, 56.3, 53.3, 50.8, 45.1, 37.1, 28.8, 25.7, 21.5, 13.7. LRMS (ESI+) m/z calcd for C₂₈H₃₃FN₃O₃S⁺ [M+H]⁺ 510.21. found 510.72.

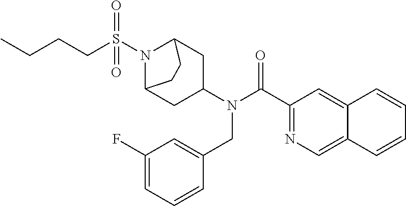 [SMILES: CCCCS(=O)(=O)N1C2CCC1CC(N(Cc1cccc(F)c1)C(=O)c1cc3ccccc3cn1)C2]

### N-benzyl-N-(3-(butylsulfonyl)-3-azabicyclo[3.1.1]heptan-6-yl)isoquinoline-3-carboxamide (116)

Yield 29%, Light-brown solid, ¹H NMR (600 MHz, Methanol-d₄) δ 9.27 (s, 1H), 8.18-8.12 (m, 2H), 7.99 (d, J=8.2 Hz, 1H), 7.84-7.75 (m, 2H), 7.29-6.96 (m, 5H), 3.82 (s, 1H), 3.76-3.56 (m, 4H), 3.31 (s, 2H), 3.21 (s, 2H), 2.99 (s, 2H), 1.94 (s, 1H), 1.84 (q, J=7.7 Hz, 2H), 1.62 (s, 1H), 1.54 (q, J=7.5 Hz, 2H), 0.97 (t, J=7.4 Hz, 3H). ¹³C NMR (151 MHz, MeOD) δ 165.7, 151.7, 137.1, 135.7, 131.3, 129.9, 129.0, 128.4, 127.5, 127.3, 127.2, 126.9, 124.0, 54.3, 53.6, 51.7, 51.2, 45.4, 36.0, 35.8, 25.0, 21.4, 12.6. LRMS (ESI+) m/z calcd for C₂₇H₃₂N₃O₃S⁻ [M+H]⁺ 478.20. found 478.67.

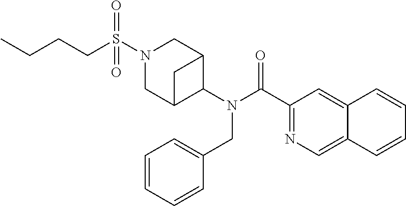 [SMILES: CCCCS(=O)(=O)N1CC2CC(C1)C2N(Cc1ccccc1)C(=O)c1cc2ccccc2cn1]

### N-benzyl-N-(2-(butylsulfonyl)-2-azabicyclo[2.2.1]heptan-5-yl)isoquinoline-3-carboxamide (117)

Yield 53%, Light-brown solid, ¹H NMR (600 MHz, Methanol-d₄) δ 9.27 (s, 1H), 8.13 (d, J=8.2 Hz, 1H), 7.91 (s, 2H), 7.80 (t, J=7.6 Hz, 1H), 7.74 (t, J=7.6 Hz, 1H), 7.29 (d, J=8.6 Hz, 3H), 7.22-7.12 (m, 2H), 5.23 (s, 1H), 4.79-4.76 (m, 1H), 4.69 (s, 3H), 4.12 (s, 1H), 3.60 (d, J=9.7 Hz, 1H), 3.40 (dt, J=9.2, 2.7 Hz, 1H), 3.15-3.02 (m, 2H), 1.93-1.89 (m, 2H), 1.79-1.74 (m, 3H), 1.48 (q, J=7.5 Hz, 2H), 0.96 (t, J=7.4 Hz, 3H). ¹³C NMR (151 MHz, Methanol-d₄) δ 172.3, 151.7, 147.9, 138.2, 135.7, 131.4, 129.6, 128.7, 128.6, 128.3, 127.6, 126.9, 125.5, 119.9, 111.1, 59.9, 59.8, 50.1, 42.8, 37.0, 34.3, 25.2, 21.2, 12.6. LRMS (ESI+) m/z calcd for C₂₇H₃₂N₃O₃S⁺ [M+H]⁺ 478.20. found 478.60.

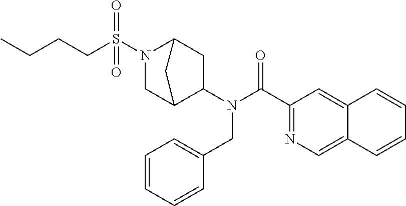 [SMILES: CCCCS(=O)(=O)N1CC2CC1CC2N(Cc1ccccc1)C(=O)c1cc2ccccc2cn1]

### N-benzyl-N-(2-(butylsulfonyl)-2-azabicyclo[2.2.2]octan-5-yl)isoquinoline-3-carboxamide (118)

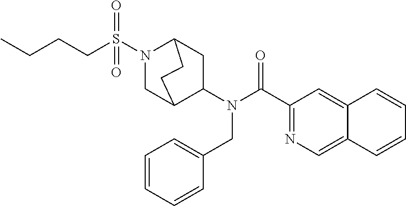 [SMILES: CCCCS(=O)(=O)N1CC2CCC1CC2N(Cc1ccccc1)C(=O)c1cc2ccccc2cn1]

Yield 29%, Pale yellow solid, H NMR (600 MHz, CDCl₃) δ 9.18 (s, 1H), 8.12-7.56 (m, 5H), 7.27-7.17 (m, 5H), 5.24-5.09 (m, 1H), 4.97-4.69 (m, 2H), 3.83-3.80 (m, 1H), 3.30 (br, 1H), 2.95-2.88 (m, 2H), 2.52-2.20 (m, 2H), 2.13-1.55 (m, 8H), 1.46-1.38 (m, 2H), 0.98-0.82 (m, 3H).

¹³C NMR (151 MHz, CDCl₃) δ 171.1, 151.2, 148.8, 135.9, 131.0, 128.7, 128.6, 127.5, 127.3, 127.1, 126.9, 125.9, 121.1, 51.3, 51.1, 50.1, 46.9, 46.5, 45.4, 31.9, 27.8, 25.4, 25.4, 21.7, 19.8, 13.6. LRMS (ESI+) m/z calcd for C₂₈H₃₄N₃O₃S⁺ [M+H]⁺ 492.22. found 492.73.

### N-benzyl-N-(3-(butylsulfonyl)-3-azabicyclo[3.2.1]octan-8-yl)isoquinoline-3-carboxamide (119)

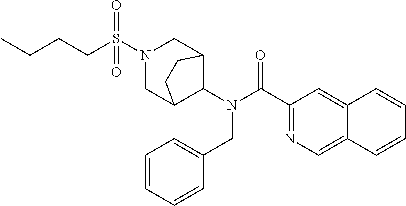 [SMILES: CCCCS(=O)(=O)N1CC2CCC(C1)C2N(Cc1ccccc1)C(=O)c1cc2ccccc2cn1]

Yield 45%, Pale yellow solid, ¹H NMR (600 MHz, CDCl₃) δ 9.23-9.14 (m, 1H), 8.06 (s, 1H), 7.98 (d, J=8.1 Hz, 1H), 7.85 (d, J=8.2 Hz, 1H), 7.74-7.68 (m, 1H), 7.68-7.62 (m, 1H), 7.29-7.25 (m, 2H), 7.21-7.06 (m, 3H), 4.93 (s, 2H), 4.28 (s, 1H), 3.61-3.50 (m, 2H), 3.08-2.95 (m, 2H), 2.93-2.84 (m, 2H), 2.60 (s, 2H), 2.04-1.95 (m, 2H), 1.90-1.83 (m, 2H), 1.77-1.72 (, 2H), 1.42 (q, J=7.5 Hz, 2H), 0.93 (t, J=7.4 Hz, 3H). ¹³C NMR (151 MHz, CDCl₃) δ 172.2, 151.2, 148.4, 139.2, 135.9, 131.0, 128.8, 128.6, 127.6, 127.4, 127.0, 125.5, 121.8, 67.1, 57.6, 52.9, 49.6, 46.5, 35.4, 26.9, 25.1, 21.8, 13.6. LRMS (ESI+) m/z calcd for C₂₈H₃₄N₃O₃S⁺ [M+H]⁺ 492.22. found 492.58.

### N-benzyl-N-(1-(butylsulfonyl)-2-methylpiperidin-4-yl)isoquinoline-3-carboxamide (120)

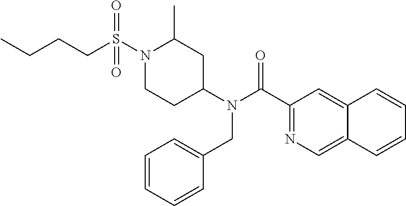 [SMILES: CCCCS(=O)(=O)N1CCC(N(Cc2ccccc2)C(=O)c2cc3ccccc3cn2)CC1C]

Yield 27%, Pale yellow solid, H NMR (600 MHz, CDCl₃) δ 9.23-9.15 (m, 1H), 8.19-7.90 (m, 2H), 7.88-7.60 (m, 2H), 7.48-7.31 (m, 2H), 7.28-7.17 (m, 4H), 4.84-4.75 (m, 2H), 4.64-3.96 (m, 2H), 3.93-3.74 (m, 1H), 3.63-2.45 (m, 4H), 1.94-1.92 (m, 2H), 1.83-1.63 (m, 3H), 1.46-1.25 (m, 6H), 0.98-0.82 (m, 3H). ¹³C NMR (151 MHz, CDCl₃) δ 169.9, 151.3, 148.5, 138.8, 136.0, 131.1, 128.5, 128.4, 127.6, 127.3, 127.0, 121.0, 52.7, 48.7, 45.0, 40.0, 37.2, 29.7, 29.3, 25.6, 21.6, 13.6. LRMS (ESI+) m/z calcd for C₂₇H₃₄N₃O₃S⁺ [M+H]⁺ 480.22. found 480.59.

### N-benzyl-N-(1-(butylsulfonyl)-3-methylpiperidin-4-yl)isoquinoline-3-carboxamide (121)

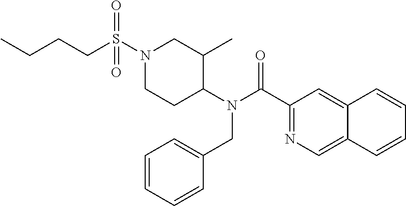 [SMILES: CCCCS(=O)(=O)N1CCC(N(Cc2ccccc2)C(=O)c2cc3ccccc3cn2)C(C)C1]

Yield 51%, Pale yellow solid, ¹H NMR (600 MHz, CDCl₃) δ 9.30-9.04 (m, 1H), 8.16-7.57 (m, SH), 7.47-7.27 (m, 2H), 7.23-7.01 (m, 3H), 5.45-5.42 (m, 1H), 4.87-4.26 (m, 2H), 3.88-3.62 (m, 2H), 2.93-2.45 (m, 5H), 2.16-2.08 (m, 1H), 1.83-1.52 (m, 3H), 1.47-1.35 (m, 2H), 1.33-1.13 (m, 3H), 0.99-0.85 (m, 3H). ¹³C NMR (151 MHz, CDCl₃) δ 170.9, 162.6, 151.2, 148.7, 135.8, 130.9, 128.5, 128.4, 127.6, 126.9, 125.9, 120.9, 63.0, 56.0, 51.9, 46.2, 36.5, 31.5, 25.1, 21.7, 13.6, 11.9. LRMS (ESI+) m/z calcd for C₂₇H₃₄N₃O₃S*[M+H]⁺ 480.22. found 480.67.

### N-benzyl-N-(1-(butylsulfonyl)-3-ethylpiperidin-4-yl)isoquinoline-3-carboxamide (122)

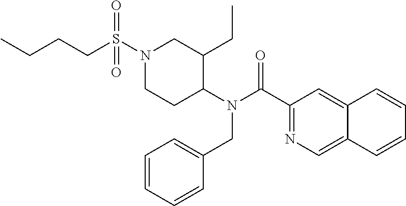 [SMILES: CCCCS(=O)(=O)N1CCC(N(Cc2ccccc2)C(=O)c2cc3ccccc3cn2)C(CC)C1]

Yield 60%, Pale yellow solid, ¹H NMR (600 MHz, CDCl₃) δ 9.33-9.02 (m, 1H), 8.16-7.57 (m, 5H), 7.42-7.02 (m, 5H), 5.54 (d, J=17.1 Hz, 1H), 4.77-4.74 (m, 1H), 4.50-4.25 (m, 1H), 3.95-3.63 (m, 2H), 3.18-2.19 (m, 5H), 2.15-2.08 (m, 1H), 1.90-1.61 (m, 5H), 1.47-1.39 (m, 2H), 1.14-0.82 (m, 6H). ¹³C NMR (151 MHz, CDCl₃) δ 170.9, 151.1, 148.8, 138.4, 135.8, 131.0, 128.5, 128.5, 128.4, 127.5, 126.8, 121.4, 61.7, 56.8, 49.1, 47.4, 46.2, 40.8, 31.1, 25.1, 21.8, 17.2, 13.6, 11.9. LRMS (ESI+) m/z calcd for C₂₈H₃₆N₃O₃S⁺ [M+H]⁺ 494.23. found 494.73.

### N-benzyl-N-(1-(butylsulfonyl)-3-fluoropiperidin-4-yl)isoquinoline-3-carboxamide (123)

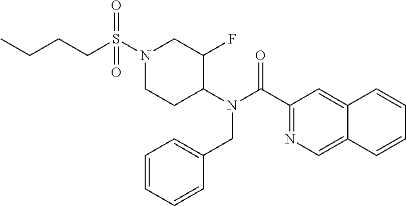 [SMILES: CCCCS(=O)(=O)N1CCC(N(Cc2ccccc2)C(=O)c2cc3ccccc3cn2)C(F)C1]

Yield 41%, Pale yellow solid, ¹H NMR (600 MHz, CDCl₃) δ 9.24-9.10 (m, 1H), 8.33-7.99 (m, 1H), 7.98-7.61 (m, 4H), 7.47-7.27 (m, 2H), 7.25-7.03 (m, 3H), 5.45-5.07 (m, 2H), 5.05-4.75 (m, 1H), 4.75-4.37 (m, 1H), 4.29-3.96 (m, 1H), 3.96-3.72 (m, 1H), 3.08-2.79 (m, 3H), 2.26-2.04 (m, 1H), 1.93-1.47 (m, 4H), 1.44 (dt, J=8.5, 3.3 Hz, 2H), 0.94 (t, J=7.3 Hz, 3H). ¹³C NMR (151 MHz, CDCl₃) δ 170.8, 151.2, 147.9, 139.1, 135.7, 130.9, 128.5, 128.3, 127.6, 126.8, 126.4, 121.3, 90.9, 89.7, 88.7, 87.5, 57.8, 54.2, 51.4, 49.8, 49.2, 45.1, 25.3, 24.8, 21.6, 13.6. LRMS (ESI+) m/z calcd for C₂₆H₃₁FN₃O₃S⁻ [M+H]⁺ 484.19. found 484.59.

### 1-benzyl-1-(8-butyl-8-azabicyclo[3.2.1]octan-3-yl)-3-(3,4-dichlorophenyl)urea (124)

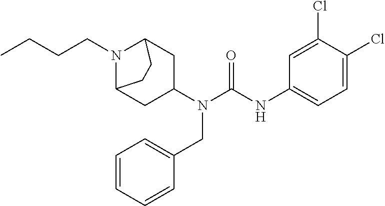 [SMILES: CCCCN1C2CCC1CC(N(Cc1ccccc1)C(=O)Nc1ccc(Cl)c(Cl)c1)C2]

Yield 45%, Light green solid, ¹H NMR (600 MHz, CDCl₃) δ 7.83 (s, 1H), 7.37 (t, J=7.5 Hz, 2H), 7.35-7.24 (m, 5H), 5.14 (s, 1H), 4.53 (s, 2H), 3.67 (s, 2H), 2.75-2.46 (m, 4H), 2.19 (s, 2H), 2.02-1.50 (m, 7H), 1.38 (q, J=7.5 Hz, 2H), 0.96 (t, J=7.3 Hz, 3H). ¹³C NMR (151 MHz, CDCl₃) δ 155.1, 139.3, 132.2, 130.0, 129.0, 127.6, 126.4, 125.6, 121.4, 119.3, 58.1, 52.1, 48.3, 46.1, 35.1, 29.0, 20.3, 13.8. LRMS (ESI+) m/z calcd for C₂₅H₃₂Cl₂N₃O [M+H]⁺ 460.18. found 460.69.

### 1-benzyl-1-((1R,5R)-8-butyl-8-azabicyclo[3.2.1]octan-3-yl)-3-(3,4-dichlorophenyl)urea (Exo Form) (125)

Yield 55%, White powder solid, ¹H NMR (400 MHz CDCl₃) δ 7.48 (s, 1H), 7.41-7.35 (m, 2H), 7.33-7.27 (m, 3H), 7.23 (d, J=8.7 Hz, 1H), 6.90 (dd, J=8.8, 2.5 Hz, 1H), 6.49 (s, 1H), 5.02 (s, 1H), 4.84 (s, 2H), 3.87 (s, 2H), 2.83-2.76 (m, 4H), 2.28-2.07 (m, 4H), 1.88-1.81 (m, 4H), 1.37 (q, J=7.5 Hz, 2H), 0.94 (t, J=7.3 Hz, 3H). ¹³C NMR (100 MHz, CDCl₃) δ 155.6, 138.3, 137.1, 132.5, 130.1, 129.3, 128.0, 126.3, 126.1, 121.4, 119.1, 61.2, 51.7, 46.5, 44.7, 32.7, 26.9, 24.9, 20.2, 13.6. LRMS (ESI+) m/z calcd for C₂₅H₃₂Cl₂N₃O⁺ [M+H]⁺ 460.18. found 484.59.

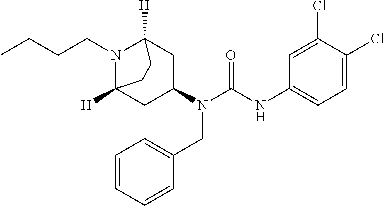

### 1-benzyl-1-((1R,5R)-8-butyl-8-azabicyclo[3.2.1]octan-3-yl)-3-(3,4-dichlorophenyl)urea (Endo Form) (126)

Yield 54%, White powder solid, ¹H NMR (400 MHz CDCl₃) δ 7.84 (s, 1H), 7.37-7.28 (m, 3H), 7.29-7.24 (m, 4H), 5.15 (s, 1H), 4.51 (s, 2H), 3.67 (s, 2H), 2.67-2.59 (m, 4H), 2.20-2.16 (m, 2H), 1.89-1.55 (m, 6H), 1.36 (q, J=7.5 Hz, 2H), 0.94 (t, J=7.4 Hz, 3H). ¹³C NMR (100 MHz, CDCl₃) δ 155.1, 139.3, 138.4, 132.2, 130.0, 129.0, 127.5, 126.4, 125.5, 121.4, 119.3, 58.1, 52.1, 47.6, 46.0, 35.0, 28.9, 20.3, 13.7. LRMS (ESI+) m/z calcd for C₂₅H₃₂Cl₂N₃O⁺ [M+H]⁺ 460.18. found 484.59.

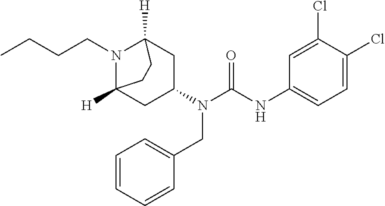

### 1-benzyl-1-(1-butyl-2-methylpiperidin-4-yl)-3-(3,4-dichlorophenyl)urea (127)

Yield 35%, White powder solid, ¹H NMR (400 MHz, CDCl₃) δ 7.46-7.38 (m, 3H), 7.37-7.33 (m, 1H), 7.31 (dd, 1=8.2, 1.5 Hz, 2H), 7.23 (d, J=8.7 Hz, 1H), 6.88 (dd, J=8.7, 2.6 Hz, 1H), 6.30 (s, 1H), 4.65 (s, 1H), 4.55 (s, 2H), 3.33 (s, 1H), 3.10-2.47 (m, 5H), 1.91 (s, 2H), 1.81-1.58 (m, 3H), 1.38-1.32 (m, 5H), 0.96 (t, J=7.4 Hz, 3H). ¹³C NMR (100 MHz, CDCl₃) δ 155.4, 138.3, 136.8, 132.5, 130.2, 129.5, 128.3, 126.2, 126.1, 121.3, 118.9, 57.4, 52.5, 51.5, 49.3, 46.2, 36.1, 30.3, 28.5, 20.5, 13.7. LRMS (ESI+) m/z calcd for C₂₄H₃₂Cl₂N₃O⁺ [M+H]⁺ 448.18. found 448.2.

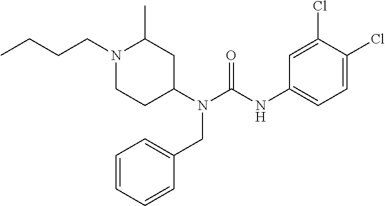 [SMILES: CCCCN1CCC(N(Cc2ccccc2)C(=O)Nc2ccc(Cl)c(Cl)c2)CC1C]

### 1-benzyl-1-(1-butyl-3-fluoropiperidin-4-yl)-3-(3,4-dichlorophenyl)urea (118)

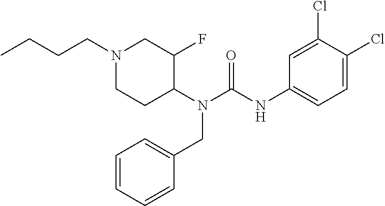 [SMILES: CCCCN1CCC(N(Cc2ccccc2)C(=O)Nc2ccc(Cl)c(Cl)c2)C(F)C1]

Yield 66%, White powder solid, ¹H NMR (400 MHz, CDCl₃) δ 7.45-7.37 (m, 3H), 7.37-7.29 (m, 3H), 7.22 (d, J=8.7 Hz, 1H), 6.86 (dd, J=8.7, 2.5 Hz, 1H), 6.36 (s, 1H), 4.96 (d, J=48 Hz, 1H), 4.81-4.59 (m, 2H), 4.59-4.44 (m, 1H), 3.34-3.21 (m, 1H), 3.06-3.03 (m, 1H), 2.45-2.24 (m, 3H), 2.23-2.19 (m, 2H), 1.74-1.66 (m, 1H), 1.53-1.41 (m, 2H), 1.37-1.25 (m, 2H), 0.91 (t, J=7.3 Hz, 3H). ¹³C NMR (100 MHz, CDCl₃) δ 155.6, 138.3, 137.4, 132.5, 130.2, 129.5, 128.1, 126.2, 126.0, 121.3, 118.9, 91.6, 89.8, 57.9, 56.7, 56.5, 54.4, 54.2, 52.8, 48.1, 48.1, 29.0, 25.2, 20.7, 14.0. LRMS (ESI+) m/z calcd for C₂₃H₂₉Cl₂FN₃O⁺ [M+H]⁺ 452.15. found 484.59.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

1. A compound having a structure represented by a formula: 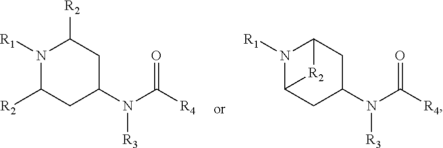
or a pharmaceutically acceptable salt thereof;
wherein R1 is alkyl, —SO₂-alkyl, or —CO-alkyl;
wherein R2 is independently H, —CH2, —CH2-CH2-, or —CH₃;
wherein R3 is alkyl or —CH2Ar; and
wherein R4 is —NH—Ar, —NH-alkyl, Ar, or alkyl.

2. The compound according to claim 1, wherein R2 is —CH2-CH2-.

3. The compound according to claim 1, wherein R4 is —NH—Ar.

4. The compound according to claim 1, wherein R4 is Ar.

5. A pharmaceutical composition comprising the compound of claim 1 or a pharmaceutically acceptable salt thereof, and a pharmaceutically acceptable carrier.

6. A method for treating a disorder characterized by uncontrolled cellular proliferation, the method comprising the step of administering to a subject in need thereof a therapeutically effective amount of the compound or a pharmaceutically acceptable salt thereof of claim 1.

7. The method of claim 6, wherein the disease or disorder is a cancer.

8. A method for treating a disorder characterized by uncontrolled cellular proliferation, the method comprising the step of administering to a subject in need thereof a therapeutically effective amount of the pharmaceutical composition of claim 5.

9. The method of claim 8, wherein the disease or disorder is a cancer.
